(19) United States
(12) Patent Application Publication
(10) Pub. No.: US 2016/0017349 A1
(43) Pub. Date: Jan. 21, 2016

(54) MAIZE MICRORNA SEQUENCES AND TARGETS THEREOF FOR AGRONOMIC TRAITS

(71) Applicant: PIONEER HI-BRED INTERNATIONAL, INC., Johnston, IA (US)

(72) Inventors: Mule Ayele, JOHNSTON, IA (US); KEVIN HAYES, URBANDALE, IA (US); CARL SIMMONS, DES MOINES, IA (US); SOBHANA SIVASANKAR, ADEL, IA (US); JOHN VAN HEMERT, Johnston, IA (US); BING-BING WANG, JOHNSTON, IA (US)

(21) Appl. No.: 14/772,816

(22) PCT Filed: Mar. 13, 2014
(86) PCT No.: PCT/US14/26380

§ 371 Date: Sep. 4, 2015

Related U.S. Application Data
(60) Provisional application No. 61/786,368, filed on Mar. 15, 2013.

(51) Int. Cl. C12N 15/82

(52) U.S. Cl. CPC C12N 15/8218; C12N 15/8261; C12N 15/8273

(57) ABSTRACT

Methods and compositions for maize target gene suppression and improving an agronomic trait through microRNAs or target gene modulation are disclosed. Polynucleotide constructs useful for gene silencing, or upregulation or modulation as well as cells, plants and seeds comprising the polynucleotides and methods for using microRNAs to silence a target gene are also described.

### FIELD

The field relates generally to plant molecular biology in relation to methods of suppressing gene expression.

### BACKGROUND

MicroRNAs (miRNAs) were first identified only a few years ago, but already it is clear that they play an important role in regulating gene activity. These short nucleotide noncoding RNAs have the ability to hybridize via base-pairing with specific target mRNAs and down-regulate the expression of these transcripts, by mediating either RNA cleavage or translational repression. Recent studies have indicated that miRNAs have important functions during development. In plants, they have been shown to control a variety of developmental processes including flowering time, leaf morphology, organ polarity, floral morphology, and root development. Given the established regulatory role of miRNAs, it is likely that they are also involved in the control of some of the major crop traits such drought tolerance and disease resistance.

Improving crop plants for water use efficiency or nitrogen use efficiency and yield, among others, are needed to improve crop productivity necessary to feed a growing population. MicroRNAs are key regulators of plant processes, and thus effort to develop the use of microRNAs to improving crop plants is of high interest and potential value. They are believed to regulate diverse processes in plants from development to environmental adaptations.

### BRIEF DESCRIPTION OF THE TABLES

Table 1 lists the SEQ ID NOS of the microRNA core sequences (Column A), the microRNA precursor genes (Column B) and the corresponding microRNA target genes (Column C) for the microRNA sequences of Column A. In column C, the transcript SEQ ID NO and any corresponding peptide SEQ ID NO for each target gene are listed separated by a comma (,). Every target gene transcript and its associated peptide SEQ ID NOs are separated by a semi-colon (;) in Column C from another transcript-peptide pair. If a particular transcript does not have an associated peptide sequence, then the designation “No_Pept” was used (see e.g., for microRNA SEQ ID NO: 32). The sequences for the SEQ ID NOs listed in Columns A-C are provided in the accompanying sequence listing, incorporated herein by reference in its entirety. As shows in Table 1, a particular core microRNA may have more than precursor gene and more than one target gene.

Table 2 lists the relative trait values for drought (Column D), nitrogen use efficiency (nitrogen; Column E), and yield (Column F) with respect to each target gene (Column A) and the translated peptide sequence (Column B) for the target gene. The relevant traits are indicated as such (Column C). For example, some target genes have high relative trait values for all the three referenced traits. Some target genes are represented under only of the traits (e.g., drought or nitrogen or yield).

### BRIEF DESCRIPTION OF THE SEQUENCE LISTING

A sequence listing is provided herewith in electronic medium. The contents of the sequence listing are hereby incorporated by reference in compliance with 37 CFR 1.52(e).

SEQ ID NOS: 1-197 are core microRNA sequences. SEQ ID NOS: 198-1126 are microRNA precursor genes. SEQ ID NOS: 1127-2495 are microRNA target gene nucleotide sequences (transcripts). SEQ ID NOS: 2496-3804 are microRNA target gene translated amino acid sequences (peptides).

### SUMMARY

A method of improving an agronomic trait of a maize plant, the method includes providing a transgenic maize plant comprising in its genome a recombinant DNA having at least one DNA element for modulating the expression of at least one target gene, wherein the at least one DNA element is selected from the group consisting of nucleotide sequences that are at least 90% identical to SEQ ID NOS: 1-197. In an embodiment, the agronomic trait is drought tolerance. In an embodiment, the agronomic trait is nitrogen use efficiency. In an embodiment, the agronomic trait is yield increase.

In an embodiment, the DNA elements whose sequences are disclosed herein, for example in Table 1 and in the accompanying Sequence Listing, modulate the expression of a target gene sequence selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703. In an embodiment, the DNA element modulates the expression of a gene sequence encoding a target peptide sequence selected from the group consisting of SEQ ID NOS: 2497, 2499, 2505, 2507, 2514, 2516, 2526, 2530, 2536, 2542, 2623, 2634, 2676, 2753, 2831, 2832, 2888, 2892, 2895, 2943, 2947, 2955, 2975, and 3054.

A method of improving an agronomic trait of a maize plant, the method includes providing a transgenic maize plant comprising in its genome a recombinant DNA for modulating the expression of at least one target gene, wherein the target gene sequence is selected from the group consisting of SEQ ID NOS: 1127-2495. In an embodiment, the target gene sequence is selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703 and wherein the agronomic trait is one of drought tolerance, nitrogen use efficiency or yield. In an embodiment, the target gene sequence is selected from the group consisting of SEQ ID NOS: 1168, 1178, 1179, 1185, 1194, 1220, 1710, 1716, 1733, 1738, 1771, 1784, 1795, 1807, 1823, 1872, 1892, 1926, 1936, 1937, 1938, 1942, 1970, 2001, 2003, 2006, 2026, 2074, 2105, 2109, 2110, 2130, 2145, 2152, 2174, 2175, 2189, 2192, 2199, 2200, 2202, 2240, 2245, 2246, 2291, 2299, 2310, 2313, 2340, 2341, 2371, 2412, 2413, 2414, 2417, 2429, 2430, 2431, 2443, 2468 and wherein the agronomic trait is one of nitrogen use efficiency or yield.

In an embodiment, the target gene sequence for modulation by a DNA element encoding an interfering RNA is selected from the group consisting of SEQ ID NOS: 1135, 1137, 1141, 1142, 1143, 1146, 1153, 1154, 1160, 1164, 1166, 1169, 1183, 1190, 1192, 1195, 1208, 1231, 1255, 1256, 1258, 1267, 1275, 1278, 1279, 1283, 1290, 1299, 1307, 1322, 1336, 1339, 1342, 1347, 1353, 1355, 1361, 1362, 1363, 1373, 1378, 1409, 1415, 1430, 1431, 1432, 1437, 1448, 1449, 1452, 1453, 1468, 1487, 1498, 1505, 1552, 1562, 1575, 1615, 1643, 1655, 1662, 1664, 1680, 1684 and wherein the agronomic trait is one of drought tolerance or yield.

A method of improving an agronomic trait of a maize plant, the method includes providing a transgenic maize plant comprising in its genome a recombinant DNA for modulating the expression of at least one target gene, wherein the target gene sequence encodes a target polypeptide sequence selected from the group consisting of SEQ ID NOS: 2496-3804. In an embodiment, the target polypeptide sequence is selected from the group consisting of SEQ ID NOS: 2497, 2499, 2505, 2507, 2514, 2516, 2526, 2530, 2536, 2542, 2623, 2634, 2676, 2753, 2831, 2832, 2888, 2892, 2895, 2943, 2947, 2955, 2975, and 3054 and wherein the agronomic trait is one of drought tolerance, nitrogen use efficiency or yield. In an embodiment, the target polypeptide sequence is selected from the group consisting of SEQ ID NOS: 2498, 2501, 2503, 2524, 2568, 2602, 2606, 2613, 2618, 2629, 2632, 2640, 2652, 2660, 2664, 2685, 2695, 2720, 2742, 2752, 2757, 2759, 2770, 2780, 2790, 2795, 2796, 2797, 2799, 2802, 2811, 2814, 2818, 2819, 2820, 2822, 2833, 2834, 2835, 2836, 2837, 2842, 2847, 2849, 2857, 2884, 2918, 2936, 2939, 2942, 2948, 2954, 2956, 2957, 2958, 2959, 2965, 2966, 2967, 2983, 2995, 2996, 3035, 3037, 3055, 3058 and wherein the agronomic trait is one of drought tolerance or nitrogen use efficiency.

In an embodiment, the target gene sequence that is modulated by a nucleic acid encodes a target peptide sequence selected from the group consisting of SEQ ID NOS: 2537, 2547, 2548, 2554, 2563, 2589, 3061, 3067, 3084, 3089, 3121, 3134, 3145, 3156, 3172, 3220, 3239, 3271, 3281, 3282, 3283, 3287, 3311, 3287, 3341, 3344, 3364, 3409, 3438, 3461, 3476, 3482, 3503, 3504, 3518, 3521, 3528, 3529, 3531, 3568, 3573, 3574, 3618, 3625, 3636, 3639, 3666, 3667, 3696, 3731, 3732, 3733, 3734, 3743, 3744, 3756, 3780, and wherein the agronomic trait is one of nitrogen use efficiency or yield.

An isolated polynucleotide includes a microRNA selected from the group consisting of SEQ ID NOS: 1-197, wherein the microRNA modulates the expression of a target gene in maize involved in an agronomic trait, the target gene selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703.

A recombinant DNA construct includes the polynucleotides disclosed herein, for example, the polynucleotides encoding the miRNAs of Table 1, wherein the DNA construct includes a plant expressible regulatory element.

An isolated polynucleotide comprising a microRNA selected from the group consisting of SEQ ID NOS: 1-197, wherein the microRNA modulates the expression of a target gene in maize involved in an agronomic trait, the target gene selected from the group consisting of SEQ ID NOS: 1168, 1178, 1179, 1185, 1194, 1220, 1710, 1716, 1733, 1738, 1771, 1784, 1795, 1807, 1823, 1872, 1892, 1926, 1936, 1937, 1938, 1942, 1970, 2001, 2003, 2006, 2026, 2074, 2105, 2109, 2110, 2130, 2145, 2152, 2174, 2175, 2189, 2192, 2199, 2200, 2202, 2240, 2245, 2246, 2291, 2299, 2310, 2313, 2340, 2341, 2371, 2412, 2413, 2414, 2417, 2429, 2430, 2431, 2443, 2468 and wherein the agronomic trait is one of nitrogen use efficiency or yield.

In an embodiment, the transgenic maize plant includes the DNA constructs disclosed herein. In an embodiment, the transgenic seed includes the DNA constructs disclosed herein.

A transgenic maize plant, wherein the expression of a target gene is reduced compared to a control plant, the target gene sequence is selected from the group consisting of SEQ ID NOS: 1127-2495, and wherein the transgenic maize plant exhibits drought tolerance, nitrogen use efficiency, or increased yield or a combination thereof.

A transgenic maize plant, wherein the expression of a target gene is reduced compared to a control plant, the target gene sequence is 90% identical to a polynucleotide sequence selected from the group consisting of SEQ ID NOS: 1127-2495, and wherein the transgenic maize plant exhibits drought tolerance, nitrogen use efficiency, or increased yield or a combination thereof.

A recombinant DNA construct includes a microRNA precursor gene selected from the group consisting of SEQ ID NOS: 198-1126 or a fragment thereof to modulate the expression of a target gene. In an embodiment, the DNA constructs disclosed herein modulate the expression of a target gene selected from the group consisting of SEQ ID NOS: 1127-2495, and wherein the target gene modulates drought tolerance, nitrogen use efficiency, or increased yield or a combination thereof.

A method of developing a maize plant, the method includes selecting a maize plant using marker assisted selection from a plurality of maize plants by detecting a molecular marker, wherein the molecular marker is derived from a polynucleotide sequence selected from the group consisting of (i) SEQ ID NOS: 198-1126 or a complement thereof or (ii) SEQ ID NOS: 1127-2495 or a complement thereof. In an embodiment, a maize plant produced by the method of marker assisted selection is disclosed herein. In an embodiment, a maize plant cell produced by the method of marker assisted selection is disclosed herein. In an embodiment, the maize seed produced by the method of marker assisted selection is disclosed herein.

An artificial or a synthetic nucleic acid molecule encoding a single stranded or double stranded RNA molecule is disclosed, wherein the nucleic acid molecule is designed based on the complementarity to one of (i) the miRNA sequences of SEQ ID NOS: 1-197; (ii) the miRNA precursor genes of SEQ ID NOS: 198-1126; or (iii) the target genes of SEQ ID NOS: 1127-2495.

### DETAILED DESCRIPTION

Regulatory activity of microRNAs (miRNA) is specific towards certain sets of genes depending on the sequence similarity of the target genes. The site of action for these miRNAs within the target gene can vary, and can affect for example, promoter function, mRNA stability or translation, thus affecting the overall expression and activity of the target genes. Often the miRNAs have negative regulatory function upon the target gene. The target genes are often regulators of a pathway or a network hub or a node, and depending upon whether they have intrinsic negative or positive regulations of the neighboring or downstream genes in their respective networks, the net effect upon the pathway-network system of the microRNA regulation can be either positive or negative.

Based on a comprehensive survey of maize microRNAs, their source genes, and the likely target genes they regulate, methods and compositions are disclosed herein that modulate gene functions and improve crop productivity through water use efficiency, or nitrogen use efficiency or yield.

Relative trait values were assigned to the various target genes depending on the likelihood of their role in association with relevant agronomic traits, such as water use efficiency (WUE, drought), nitrogen use efficiency (NUE, Nitrogen), and yield. The miRNA sequences and the corresponding target gene sequences establish relationships among the miRNAs and their target genes for trait efficacy. These miRNAs and/or their target genes can be used, for example by recombinant technology to induce gene suppression or as tools to enable marker-assisted selection for breeding purposes towards crop improvement.

In an embodiment, modulating the expression of the miRNA or the interaction of the miRNA with the target gene, results in improving one or more agronomic traits in the crop plants. Depending on the anti-correlated nature of the microRNAs relative to the target genes, for example, a down-regulation of a microRNA would equate to an upregulation of the target gene. Therefore, it is possible to upregulate the expression of a target gene transgenically without expressing a recombinant nucleic acid of the target encoding the target peptide. In an embodiment, for example, by changing the expression of an endogenous miRNA either through transgenic suppression methods or by engineering a site-specific change in the precursor gene for the endogenous miRNA, expression and/or activity of the corresponding target gene(s) can be modulated.

In an embodiment, to modulate the expression of one or more genes involved in a pathway or those genes that share sequence similarity, one or a few miRNAs can be expressed to affect the expression of multiple genes. For example, one microRNA (SEQ ID NO: 46) can affect the expression of a number of genes involved in drought or nitrogen or yield (see Table 1; target gene SEQ ID NOS: 1128, 1147, 1289, 1311, 1314, 1316, 1338, and others).

Methods and compositions useful for suppressing targeted sequences are disclosed. The compositions can be employed in any type of plant cell, and in other cells which comprise the appropriate processing components (e.g., RNA interference components), including invertebrate and vertebrate animal cells. The compositions and methods are based on an endogenous miRNA silencing process discovered in Arabidopsis, a similar strategy can be used to extend the number of compositions and the organisms in which the methods are used. The methods can be adapted to work in any eukaryotic cell system. Additionally, the compositions and methods described herein can be used in individual cells, cells or tissue in culture, or in vivo in organisms, or in organs or other portions of organisms.

The compositions selectively suppress the target gene by encoding a miRNA having substantial complementarity to a region of the target gene. The miRNA is provided in a nucleic acid construct which, when transcribed into RNA, is predicted to form a hairpin structure which is processed by the cell to generate the miRNA, which then suppresses expression of the target gene.

Nucleic acid sequences are disclosed that encode miRNAs from maize. Backbone hairpins containing the individual miRNA sequences are also disclosed. Constructs are described for transgenic expression of miRNAs and their backbones. Alternatively, constructs are described wherein backbone sequences and miRNA sequences are exchanged thereby altering the expression pattern of the miRNA, and its subsequent specific target gene in the transgenic host. Any miRNA can be exchanged with any other backbone to create a new miRNA/backbone hybrid.

A method for suppressing a target gene is provided. The method employs any of the constructs above, in which a miRNA is designed to identify a region of the target sequence, and inserted into the construct. Upon introduction into a cell, the miRNA produced suppresses expression of the targeted sequence. The target sequence can be an endogenous plant sequence, or a heterologous transgene in the plant.

There can also be mentioned as the target gene, for example, a gene from a plant pathogen, such as a pathogenic virus, nematode, insect, or mold or fungus.

Another aspect concerns a plant, cell, and seed comprising the construct and/or the miRNA. Typically, the cell will be a cell from a plant, but other prokaryotic or eukaryotic cells are also contemplated, including but not limited to viral, bacterial, yeast, insect, nematode, or animal cells. Plant cells include cells from monocots and dicots. The disclosure also provides plants and seeds comprising the construct and/or the miRNA.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The term “plant parts” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in plant or in a plant organ, tissue or cell culture.

The term “plant organ” refers to plant tissue or group of tissues that constitute a morphologically and functionally distinct part of a plant.

The term “genome” refers to the following: (1) the entire complement of genetic material (genes and non-coding sequences) present in each cell of an organism, or virus or organelle; (2) a complete set of chromosomes inherited as a (haploid) unit from one parent.

“Progeny” comprises any subsequent generation of a plant. Progeny will inherit, and stably segregate, genes and transgenes from its parent plant(s).

Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges recited within the specification are inclusive of the numbers defining the range and include each integer within the defined range. Amino acids may be referred to herein by either commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. Screening may be accomplished by Southern analysis of DNA, Northern analysis of mRNA expression, immunoblotting analysis of protein expression, or phenotypic analysis, among others.

This construct may comprise any combination of deoxyribonucleotides, ribonucleotides, and/or modified nucleotides. The construct may be transcribed to form an RNA, wherein the RNA may be capable of forming a double-stranded RNA and/or hairpin structure. This construct may be expressed in the cell, or isolated or synthetically produced. The construct may further comprise a promoter, or other sequences which facilitate manipulation or expression of the construct.

As used here “suppression” or “silencing” or “inhibition” are used interchangeably to denote the down-regulation of the expression of a product of a target sequence relative to its normal expression level in a wild type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild type expression level.

As used herein, “encodes” or “encoding” refers to a DNA sequence which can be processed to generate an RNA and/or polypeptide.

As used herein, “expression” or “expressing” refers to production of a functional product, such as, the generation of an RNA transcript from an introduced construct, an endogenous DNA sequence, or a stably incorporated heterologous DNA sequence. The term may also refer to a polypeptide produced from an mRNA generated from any of the above DNA precursors. Thus, expression of a nucleic acid fragment may refer to transcription of the nucleic acid fragment (e.g., transcription resulting in mRNA or other functional RNA) and/or translation of RNA into a precursor or mature protein (polypeptide).

As used herein, “heterologous” with respect to a sequence means a sequence that originates from a foreign species, or, if from the same species, is substantially modified from its native form in composition and/or genomic locus by deliberate human intervention. For example, with respect to a nucleic acid, it can be a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention.

The term “host cell” refers to a cell which contains or into which is introduced a nucleic acid construct and supports the replication and/or expression of the construct. Host cells may be prokaryotic cells such as E. coli, or eukaryotic cells such as fungi, yeast, insect, amphibian, nematode, or mammalian cells. Alternatively, the host cells are monocotyledonous or dicotyledonous plant cells. An example of a monocotyledonous host cell is a maize host cell.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into ac ell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “genome” as it applies to a plant cells encompasses not only chromosomal DNA found within the nucleus, but organelle DNA found within subcellular components (e.g., mitochondrial, plastid) of the cell.

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

As used herein, microRNA or “miRNA” refers to an oligoribonucleic acid, which regulates expression of a polynucleotide comprising the target gene. A “mature miRNA” refers to the miRNA generated from the processing of a miRNA precursor. A “miRNA template” is an oligonucleotide region, or regions, in a nucleic acid construct which encodes the miRNA. A portion of a polynucleotide construct is substantially complementary to the miRNA template and is predicted to base pair with the miRNA template. The miRNA template and a portion of the construct may form a double-stranded polynucleotide, including a hairpin structure.

As used herein, “domain” or “functional domain” refer to nucleic acid sequence(s) that are capable of eliciting a biological response in plants. A domain could refer to a portion within either individual miRNA or groups of miRNAs. Also, miRNA sequences associated with their backbone sequences could be considered domains useful for processing the miRNA into its active form. As used herein, “subdomains” or “functional subdomains” refer to subsequences of domains that are capable of eliciting a biological response in plants. A miRNA could be considered a subdomain of a backbone sequence. “Contiguous” sequences or domains refer to sequences that are sequentially linked without added nucleotides intervening between the domains.

The phrases “target sequence”, “target gene”, “target gene sequence” and “sequence of interest” may be used interchangeably. Target sequence is used to mean the nucleic acid sequence that is selected for alteration (e.g., suppression) of expression, and is not limited to polynucleotides encoding polypeptides. The target sequence comprises a sequence that is substantially or fully complementary to the miRNA. The target sequence includes, but is not limited to, RNA, DNA, or a polynucleotide comprising the target sequence. As discussed in Bartel and Bartel (2003) Plant Phys. 132:709-719, most microRNA sequences are 20-22 nucleotides with anywhere from 0-3 mismatches when compared to their target sequences.

It is understood that microRNA sequences include for example, 21 nucleotide sequences, or shorter (e.g., 18, 19, 20 mer) or longer (22, 23, 24-mer) sequences. In addition, some nucleotide substitutions, particularly at the last two nucleotides of the 3′ end of the microRNA sequence, may be useful in retaining at least some microRNA function.

As used herein, “nucleic acid” means a polynucleotide and includes single or double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deosycytidylate, “G” for guanylate or deoxyguanylate, “U” for uridlate, “T” for deosythymidylate, “R” for purines (A or G), “Y” for pyrimidiens (Cor T), “K” for G or T, “H” for A or C or T, “I” for inosine, and “N” for any nucleotide.

By “nucleic acid library” is meant a collection of isolated DNA or RNA molecules which comprise and substantially represent the entire transcribed fraction of a genome of a specified organism or of a tissue from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage of at least two sequences. Operably linked includes linkage between a promoter and a second sequence, wherein the promoter sequence initiates and mediates transcription of the DNA sequence corresponding to the second sequence.

As used herein, “plant” includes plants and plant parts including but not limited to plant cells, plant tissue such as leaves, stems, roots, flowers, and seeds.

As used herein, “polypeptide” means proteins, protein fragments, modified proteins, amino acid sequences and synthetic amino acid sequences. The polypeptide can be glycosylated or not.

As used herein, “promoter” refers to a nucleic acid fragment, e.g., a region of DNA, that is involved in recognition and binding of an RNA polymerase and other proteins to initiate transcription. In other words, this nucleic acid fragment is capable of controlling transcription of another nucleic acid fragment.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the relevant factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995). Hybridization and/or wash conditions can be applied for at least 10, 30, 60, 90, 120, or 240 minutes.

The terms “reliable detection” and “reliably detected” are defined herein to mean the reproducible detection of measurable, sequence-specific signal intensity above background noise.

As used herein, “transgenic” refers to a plant or a cell which comprises within its genome a heterologous polynucleotide. Preferably, the heterologous polynucleotide is stably integrated within the genome such that the polynucleotide is passed on, or heritable, to successive generations. The heterologous polynucleotide may be integrated into the genome alone or as part of an expression construct. Transgenic is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” refers to a small nucleic acid molecule (plasmid, virus, bacteriophage, artificial or cut DNA molecule) that can be used to deliver a polynucleotide into a host cell. Vectors are capable of being replicated and contain cloning sites for introduction of a foreign polynucleotide. Thus, expression vectors permit transcription of a nucleic acid inserted therein.

Polynucleotide sequences may have substantial identity, substantial homology, or substantial complementarity to the selected region of the target gene. As used herein “substantial identity” and “substantial homology” indicate sequences that have sequence identity or homology to each other. Generally, sequences that are substantially identical or substantially homologous will have about 75%, 80%, 85%, 90%, 95%, or 100% sequence identity wherein the percent sequence identity is based on the entire sequence and is determined by GAP alignment using default parameters (GCG, GAP version 10, Accelrys, San Diego, Calif.). GAP uses the algorithm of Needleman and Wunsch (J. Mol. Biol. 48:443-453, 1970) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully or completely complementary.

Computational identification of miRNAs was accomplished from size selected small RNA libraries from leaf, drought-stressed leaf, seed, and various other tissues.

In some embodiments, the miRNA template, (i.e. the polynucleotide encoding the miRNA), and thereby the miRNA, may comprise some mismatches relative to the target sequence. In some embodiments the miRNA template has ≧1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA containing construct. In some embodiments the miRNA template has ≧1 nucleotide mismatch as compared to the miRNA construct, for example, the miRNA template can have 1, 2, 3, 4, 5, or more mismatches as compared to the miRNA construct. This degree of mismatch may also be described by determining the percent identity of the miRNA template to the complement of the miRNA construct. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA construct.

In some embodiments, the target sequence is selected from a plant pathogen. Plants or cells comprising a miRNA directed to the target sequence of the pathogen are expected to have decreased sensitivity and/or increased resistance to the pathogen. In some embodiments, the miRNA is encoded by a nucleic acid construct further comprising an operably linked promoter. In some embodiments, the promoter is a pathogen-inducible promoter.

In another embodiment, there is provided a nucleic acid construct for suppressing a target sequence. The nucleic acid construct encodes a miRNA substantially complementary to the target. In some embodiments, the nucleic acid construct further comprises a promoter operably linked to the polynucleotide encoding the miRNA. In some embodiments, the nucleic acid construct lacking a promoter is designed and introduced in such a way that it becomes operably linked to a promoter upon integration in the host genome. In some embodiments, the nucleic acid construct is integrated using recombination, including site-specific recombination. See, for example, WO 99/25821, herein incorporated by reference. In some embodiments, the nucleic acid construct is an RNA. In some embodiments, the nucleic acid construct comprises at least one recombination site, including site-specific recombination sites. In some embodiments the nucleic acid construct comprises at least one recombination site in order to facilitate integration, modification, or cloning of the construct. In some embodiments the nucleic acid construct comprises two site-specific recombination sites flanking the miRNA precursor. In some embodiments the site-specific recombination sites include FRT sites, lox sites, or att sites, including attB, attL, attP or attR sites. See, for example, WO 99/25821, and U.S. Pat. Nos. 5,888,732, 6,143,557, 6,171,861, 6,270,969, and 6,277,608, herein incorporated by reference.

In an embodiment, a DNA expression construct includes any of the isolated polynucleotides discussed herein operably linked to at least one regulatory sequence.

In an embodiment, the a plant includes in its genome the DNA expression constructs discussed herein. Such plants can be selected from the group consisting of corn, rice, sorghum, sunflower, millet, soybean, canola, wheat, barley, oat, beans, and nuts.

In an embodiment, transgenic seeds obtained from a plant includes in its genome the DNA expression constructs discussed herein. Also within the scope are transformed plant tissue or a plant cell comprising in its genome the DNA expression constructs discussed herein. In an embodiment, by-products and progeny plants obtained from such transgenic seeds.

In an embodiment, the nucleic acid construct comprises an isolated polynucleotide comprising a polynucleotide which encodes a modified plant miRNA precursor, the modified precursor comprising a first and a second oligonucleotide, wherein at least one of the first or the second oligonucleotides is heterologous to the precursor, wherein the first oligonucleotide is substantially complementary to the second oligonucleotide, and the second oligonucleotide comprises a miRNA substantially complementary to the target sequence, wherein the precursor is capable of forming a hairpin.

In some embodiments there are provided cells, plants, and seeds comprising the introduced polynucleotides, and/or produced by the methods disclosed herein. The cells include prokaryotic and eukaryotic cells, including but not limited to bacteria, yeast, fungi, viral, invertebrate, vertebrate, and plant cells. Plants, plant cells, and seeds include gynosperms, monocots and dicots, including but not limited to, for example, rice, wheat, oats, barley, millet, sorghum, soy, sunflower, safflower, canola, alfalfa, cotton, Arabidopsis, and tobacco.

As used herein, “by-products” refer to any product, fraction, or material produced from the processing of the seed. Corn kernels (seeds) are subjected to both wet and dry milling. The goal of both processes is to separate the germ, the endosperm, and the pericarp (hull). Wet milling separates the chemical constituents of corn into starch, protein, oil, and fiber fractions.

Methods and compositions useful in suppression of a target sequence and/or validation of function are disclosed. The disclosure also relates to a method for using microRNA (miRNA) mediated RNA interference (RNAi) to silence or suppress a target sequence to evaluate function, or to validate a target sequence for phenotypic effect and/or trait development. Constructs comprising small nucleic acid molecules, miRNAs, capable of inducing silencing, and methods of using these miRNAs to selectively silence target sequences are disclosed.

RNA interference refers to the process of sequence-specific post-transcriptional gene silencing in animals mediated by short interfering RNAs (siRNAs) (Fire et al., Nature 391:806 1998). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 1999). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

The presence of long dsRNAs in cells stimulates the activity of a ribonuclease III enzyme referred to as “dicer”. Dicer is involved in the processing of the dsRNA into short pieces of dsRNA known as short interfering RNAs (siRNAs) (Berstein et al., Nature 409:363 2001) and/or pre miRNAs into miRNAs. Short interfering RNAs derived from dicer activity are typically about 21 to about 23 nucleotides in length and comprise about 19 base pair duplexes (Elbashir et al., Genes Dev. 15:188 2001). Dicer has also been implicated in the excision of 21- and 22-nucleotide small temporal RNAs (stRNAs) from precursor RNA of conserved structure that are implicated in translational control (Hutvagner et al., 2001, Science 293:834). The RNAi response also features an endonuclease complex, commonly referred to as an RNA-induced silencing complex (RISC), which mediates cleavage of single-stranded RNA having sequence complementarity to the antisense strand of the siRNA duplex. Cleavage of the target RNA takes place in the middle of the region complementary to the antisense strand of the siRNA duplex (Elbashir et al., Genes Dev. 15:188 2001). In addition, RNA interference can also involve small RNA (e.g., microRNA, or miRNA) mediated gene silencing, presumably through cellular mechanisms that regulate chromatin structure and thereby prevent transcription of target gene sequences (see, e.g., Allshire, Science 297:1818-1819 2002; Volpe et al., Science 297:1833-1837 2002; Jenuwein, Science 297:2215-2218 2002; and Hall et al., Science 297:2232-2237 2002). As such, miRNA molecules are used to mediate gene silencing via interaction with RNA transcripts or alternately by interaction with particular gene sequences, wherein such interaction results in gene silencing either at the transcriptional or post-transcriptional level.

RNAi has been studied in a variety of systems. Fire et al. (Nature 391:806 1998) were the first to observe RNAi in C. elegans. Wianny and Goetz (Nature Cell Biol. 2:70 1999) describe RNAi mediated by dsRNA in mouse embryos. Hammond et al. (Nature 404:293 2000) describe RNAi in Drosophila cells transfected with dsRNA. Elbashir et al., (Nature 411:494 2001) describe RNAi induced by introduction of duplexes of synthetic 21-nucleotide RNAs in cultured mammalian cells including human embryonic kidney and HeLa cells.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 18 to about 24 nucleotides (nt) in length that have been identified in both animals and plants (Lagos-Quintana et al., Science 294:853-858 2001, Lagos-Quintana et al., Curr. Biol. 12:735-739 2002; Lau et al., Science 294:858-862 2001; Lee and Ambros, Science 294:862-864 2001; Llave et al., Plant Cell 14:1605-1619 2002; Mourelatos et al., Genes. Dev. 16:720-728 2002; Park et al., Curr. Biol. 12:1484-1495 2002; Reinhart et al., Genes. Dev. 16:1616-1626 2002). They are processed from longer precursor transcripts that range in size from approximately 70 to 200 nt, and these precursor transcripts have the ability to form stable hairpin structures.

The methods provided can be practiced in any organism in which a method of transformation is available, and for which there is at least some sequence information for the target sequence, or for a region flanking the target sequence of interest. It is also understood that two or more sequences could be targeted by sequential transformation, co-transformation with more than one targeting vector, or the construction of a DNA construct comprising more than one miRNA sequence. The methods are also implemented by a combinatorial nucleic acid library construction in order to generate a library of miRNAs directed to random target sequences. The library of miRNAs could be used for high-throughput screening for gene function validation.

General categories of sequences of interest include, for example, those genes involved in regulation or information, such as zinc fingers, transcription factors, homeotic genes, or cell cycle and cell death modulators, those involved in communication, such as kinases, and those involved in housekeeping, such as heat shock proteins.

Target sequences further include coding regions and non-coding regions such as promoters, enhancers, terminators, introns and the like, which may be modified in order to alter the expression of a gene of interest. For example, an intron sequence can be added to the 5′ region to increase the amount of mature message that accumulates (see for example Buchman and Berg, Mol. Cell Biol. 8:4395-4405 (1988); and Callis et al., Genes Dev. 1:1183-1200 (1987)).

The target sequence may be an endogenous sequence, or may be an introduced heterologous sequence, or transgene. For example, the methods may be used to alter the regulation or expression of a transgene, or to remove a transgene or other introduced sequence such as an introduced site-specific recombination site. The target sequence may also be a sequence from a pathogen, for example, the target sequence may be from a plant pathogen such as a virus, a mold or fungus, an insect, or a nematode. A miRNA could be expressed in a plant which, upon infection or infestation, would target the pathogen and confer some degree of resistance to the plant.

In plants, other categories of target sequences include genes affecting agronomic traits, insect resistance, disease resistance, herbicide resistance, sterility, grain characteristics, and commercial products. Genes of interest also included those involved in oil, starch, carbohydrate, or nutrient metabolism as well as those affecting, for example, kernel size, sucrose loading, and the like. The quality of grain is reflected in traits such as levels and types of oils, saturated and unsaturated, quality and quantity of essential amino acids, and levels of cellulose. Any target sequence could be suppressed in order to evaluate or confirm its role in a particular trait or phenotype, or to dissect a molecular, regulatory, biochemical, or proteomic pathway or network.

A number of promoters can be used, these promoters can be selected based on the desired outcome. It is recognized that different applications will be enhanced by the use of different promoters in plant expression cassettes to modulate the timing, location and/or level of expression of the miRNA. Such plant expression cassettes may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT US01/22169).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat, A. et al. (1986) Plant Sci. 47:95-102; Reina, M. et al. Nucl. Acids Res. 18(21):6426; and Kloesgen, R. B. et al. (1986) Mol. Gen. Genet. 203:237-244. Promoters that express in the embryo, pericarp, and endosperm are disclosed in U.S. Pat. No. 6,225,529 and PCT publication WO 00/12733. The disclosures each of these are incorporated herein by reference in their entirety.

In some embodiments it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the polynucleotides. Such wound-inducible promoters include potato proteinase inhibitor (pin II) gene (Ryan (1990) Ann. Rev. Phytopath. 28:425-449; Duan et al. (1996) Nature Biotech. 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Lett. 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a sequence of interest within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and rubisco can also be used. See, for example, Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus comiculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengopta-Gopalen et al. (1988) PNAS 82:3320-3324.

Transformation protocols as well as protocols for introducing nucleotide sequences into plants may vary depending on the type of plant or plant cell, i.e., monocot or dicot, targeted for transformation. Suitable methods of introducing the DNA construct include microinjection (Crossway et al. (1986) Biotechniques 4:320-334; and U.S. Pat. No. 6,300,543), sexual crossing, electroporation (Riggs et al. (1986) Proc. Natl. Acad. Sci. USA 83:5602-5606), Agrobacterium-mediated transformation (Townsend et al., U.S. Pat. No. 5,563,055; and U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); and U.S. Pat. No. 5,736,369 (meristem transformation), all of which are herein incorporated by reference.

The nucleotide constructs may be introduced into plants by contacting plants with a virus or viral nucleic acids. Generally, such methods involve incorporating a nucleotide construct within a viral DNA or RNA molecule. Further, it is recognized that useful promoters encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In some embodiments, transient expression may be desired. In those cases, standard transient transformation techniques may be used. Such methods include, but are not limited to viral transformation methods, and microinjection of DNA or RNA, as well other methods well known in the art.

The cells from the plants that have stably incorporated the nucleotide sequence may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic imparted by the nucleotide sequence of interest and/or the genetic markers contained within the target site or transfer cassette. Two or more generations may be grown to ensure that expression of the desired phenotypic characteristic is stably maintained and inherited and then seeds harvested to ensure expression of the desired phenotypic characteristic has been achieved.

In an embodiment, a method for altering expression of a stably introduced nucleotide sequence in a plant includes:
a) making a DNA expression construct comprising a stably introduced nucleotide sequence and at least one sequence capable of hybridizing to the isolated polynucleotide;
b) transforming a plant with the DNA expression construct of part (a); and
c) selecting a transformed plant which comprises the DNA expression construct of part (a) in its genome and which has altered expression of the stably introduced nucleotide sequence when compared to a plant transformed with a modified version of the DNA expression construct of part (a) wherein the modified construct lacks the sequence capable of hybridizing to the isolated polynucleotide disclosed herein.

TABLE 1
MicroRNA sequences and targets thereof

| Micro | | |
|---|---|---|
| RNA | | |
| Core | | |
| Seq. | | |
| (SEQ | | |
| ID | MicroRNA Precursor Genes (SEQ ID | MicroRNA Target Genes SEQ ID NOs (Transcript, |
| NO) | NOs) | Peptide; Transcript, Peptide) |
| 1 | 298, 659, 660 | 1379, 2742; 2368, 3693; |
| 2 | 917 | |
| 3 | 414 | |
| 4 | 537 | |
| 5 | 735 | 2001, 3287; |
| 6 | 198, 199, 200, 201, 202, 203, 273, 274, 275, 276, | 1248, 2617; 1835, 3183; |
| | 277, 278, 279, 280, 281, 282, 283, 284, 285, | |
| | 286, 287, 288, 289, 423, 426, 427, 428, 429, | |
| | 430, 431, 432, 433, 434, 435, 436, 437, 438, | |
| | 439, 440, 441, 442, 443, 444, 445, 446, 447, 448, | |
| | 449, 450, 451, 453, 454, 455, 456, 457, 547, | |
| | 548, 549, 550, 551, 552, 553, 554, 555, 556, | |
| | 557, 558, 559, 560, 561, 562, 563, 564, 565, | |
| | 566, 567, 638, 639, 640, 641, 642, 643, 644, 645, | |
| | 646, 647, 648, 649, 650, 651, 652, 653, 654, | |
| | 655, 656, 739, 740, 741, 742, 743, 744, 745, | |
| | 746, 747, 748, 749, 750, 751, 752, 753, 754, | |
| | 756, 757, 840, 841, 842, 843, 844, 845, 846, 847, | |
| | 848, 849, 850, 851, 890, 891, 892, 893, 894, | |
| | 895, 896, 897, 898, 899, 900, 901, 902, 903, | |
| | 904, 905, 906, 907, 908, 977, 978, 979, 980, | |
| | 981, 982, 983, 984, 985, 988, 1009, 1010, 1011, | |
| | 1012, 1013, 1014, 1015, 1072, 1073, 1074, | |
| | 1075, 1076, 1077, 1078, 1079, 1080 | |
| 7 | 420, 635 | 1942, 3287; 2026, 3364; 2484, 3796; |
| 8 | 5, 201, 092 | 1366, 2730; 1635, 2989; 1769, 3119; 1862, 3210; 2470, 3782; |
| 9 | 3, 554, 716, 991, 037 | 1366, 2730; 1635, 2989; 1769, 3119; 1862, 3210; 2319, 3645; 2470, |
| | | 3782; |
| 10 | 589 | 1366, 2730; 1635, 2989; 1769, 3119; 1862, 3210; 2470, 3782; |
| 11 | 208, 209, 223, 224, 225, 226, 227, 228, 229, 230, | 1366, 2730; 1635, 2989; 1769, 3119; 1862, 3210; 2470, 3782; |
| | 231, 232, 233, 234, 235, 237, 238, 239, 240, | |
| | 241, 242, 243, 244, 245, 246, 247, 248, 249, | |
| | 250, 251, 252, 253, 255, 256, 257, 258, 259, | |
| | 260, 261, 262, 263, 310, 311, 312, 313, 314, 315, | |
| | 316, 318, 319, 320, 321, 322, 323, 324, 325, | |
| | 326, 327, 328, 329, 330, 331, 332, 333, 334, | |
| | 335, 336, 337, 338, 339, 340, 341, 342, 343, | |
| | 344, 345, 346, 347, 348, 349, 350, 351, 352, 353, | |
| | 354, 356, 358, 359, 360, 361, 362, 363, 364, | |
| | 365, 366, 367, 368, 369, 370, 371, 372, 373, | |
| | 374, 375, 376, 377, 378, 379, 380, 381, 382, | |
| | 383, 384, 385, 386, 387, 389, 390, 391, 392, 393, | |
| | 394, 395, 396, 397, 398, 399, 400, 401, 402, | |
| | 403, 404, 406, 472, 473, 474, 475, 476, 477, | |
| | 478, 479, 480, 481, 482, 483, 484, 485, 486, | |
| | 487, 488, 489, 490, 491, 492, 493, 494, 495, 496, | |
| | 497, 498, 499, 500, 501, 502, 503, 504, 505, | |
| | 506, 507, 508, 509, 510, 511, 512, 513, 514, | |
| | 515, 516, 517, 518, 519, 521, 577, 578, 580, | |
| | 581, 582, 583, 584, 585, 586, 587, 588, 590, 591, | |
| | 592, 593, 594, 595, 596, 597, 598, 599, 600, | |
| | 601, 602, 603, 604, 605, 606, 607, 608, 609, | |
| | 610, 611, 612, 613, 614, 615, 616, 617, 669, | |
| | 670, 671, 672, 673, 674, 675, 676, 677, 678, 679, | |
| | 680, 681, 682, 683, 684, 685, 686, 687, 688, | |
| | 689, 690, 691, 692, 693, 694, 695, 696, 697, | |
| | 698, 700, 701, 702, 703, 704, 705, 706, 707, | |
| | 708, 709, 710, 711, 712, 713, 714, 715, 716, 717, | |
| | 772, 773, 774, 775, 776, 777, 778, 779, 780, | |
| | 781, 782, 783, 784, 785, 786, 787, 788, 789, | |
| | 790, 791, 792, 793, 794, 795, 796, 797, 798, | |
| | 799, 800, 801, 802, 803, 804, 805, 806, 807, 808, | |
| | 809, 810, 811, 812, 813, 814, 859, 860, 861, | |
| | 862, 863, 864, 865, 866, 867, 868, 869, 870, | |
| | 871, 872, 873, 874, 875, 876, 877, 878, 879, | |
| | 880, 923, 924, 925, 926, 927, 928, 929, 930, 931, | |
| | 932, 933, 934, 935, 936, 937, 938, 939, 940, | |
| | 941, 942, 943, 944, 945, 946, 947, 948, 949, | |
| | 950, 951, 952, 953, 954, 955, 956, 957, 958, | |
| | 959, 960, 1017, 1019, 1020, 1021, 1022, 1023, | |
| | 1024, 1025, 1026, 1027, 1028, 1029, 1030, | |
| | 1031, 1032, 1033, 1034, 1035, 1036, 1038, | |
| | 1039, 1040, 1041, 1042, 1043, 1044, 1045, 1046, | |
| | 1047, 1048, 1049, 1050, 1051, 1081, 1082, | |
| | 1083, 1084, 1085, 1086, 1087, 1088, 1089, | |
| | 1090, 1091, 1093, 1094, 1095, 1096, 1097, | |
| | 1098, 1099, 1100, 1101, 1102, 1103 | |
| 12 | 405 | 1366, 2730; 1635, 2989; 1769, 3119; 1862, 3210; 2470, 3782; |
| 13 | 835 | 1211, 2580; 1288, 2656; 1323, 2689; 1324, 2690; 1356, 2720; 1380, |
| | | 2743; 1391, 2754; 1447, 2808; 1853, 3201; 1879, 3227; 1910, 3255; |
| | | 1987, 3326; 2236, 3564; 1401, No_Pept; |
| 14 | 264, 357 | 1273, 2642; 1281, 2649; 1340, 2706; 1635, 2989; 1907, 3252; 2074, |
| | | 3409; 2275, 3603; 2372, 3697; 2470, 3782; |
| 15 | 308 | |
| 16 | 888 | 1205, 2574; 1221, 2590; 1628, 2982; 1661, 3014; |
| 17 | 626 | |
| 18 | 538 | 2423, No_Pept; |
| 19 | 569, 997 | 1395, 2758; 1489, 2845; 1657, 3010; 2299, 3625; 2427, 3741; |
| 20 | 665 | |
| 21 | 425 | 1660, 3013; 1676, 3028; |
| 22 | 6, 571, 016 | 1662, 3015; 1940, 3285; 2132, 3463; 2397, 3719; 2481, 3793; |
| 23 | 852 | 1662, 3015; 1690, 3042; 1940, 3285; 2132, 3463; 2397, 3719; 2481, |
| | | 3793; |
| 24 | 733 | |
| 25 | 732 | 1924, 3269; |
| 26 | 909 | 1260, 2629; 1934, 3279; |
| 27 | 291 | |
| 28 | 910 | 1212, 2581; 1344, 2708; 2105, 3438; 2253, 3581; 2269, 3597; 2464, |
| | | 3776; |
| 29 | 540 | 1662, 3015; 2132, 3463; 2249, 3577; |
| 30 | 317, 579 | 1635, 2989; 1716, 3067; 2265, 3593; 2275, 3603; 2470, 3782; |
| 31 | 73, 111, 051, 106, 110, 700, 000, 000 | 1680, 3032; 1928, 3273; |
| 32 | 542 | 1213, 2582; 2100, 3433; 2493, 3803; 2494, No_Pept; |
| 33 | 410 | |
| 34 | 218, 219, 221, 407, 408, 409, 523, 524, 526, 527, | |
| | 528, 620, 621, 622, 623, 624, 625, 723, 724, | |
| | 725, 727, 820, 821, 822, 823, 824, 855, 918, | |
| | 919, 920, 1000, 1001, 1002, 1057, 1058, 1060, | |
| | 1061, 1062 | |
| 35 | 856 | 2240, 3568; |
| 36 | 729, 966 | |
| 37 | 267 | |
| 38 | 881 | |
| 39 | 719 | 1150, 2519; 1151, 2520; 1179, 2548; 1183, 2552; 1277, 2645; 1473, |
| | | 2833; 1588, 2943; 1643, 2997; 1732, 3083; 1828, 3177; 1876, 3224; |
| | | 2009, 3347; 2158, 3488; 2294, 3620; 2448, 3761; |
| 40 | 815 | 1150, 2519; 1151, 2520; 1179, 2548; 1183, 2552; 1277, 2645; 1473, |
| | | 2833; 1588, 2943; 1643, 2997; 1732, 3083; 1773, 3123; 1828, 3177; |
| | | 1876, 3224; 2009, 3347; 2079, 3414; 2158, 3488; 2294, 3620; 2334, |
| | | 3660; 2375, 3699; 2448, 3761; 2471, 3783; |
| 41 | 886 | 1166, 2535; 1255, 2624; 1280, 2648; 1336, 2702; 1464, 2824; 1487, |
| | | 2843; 1550, 2905; 1611, 2965; 1630, 2984; 1778, 3128; 1975, 3316; |
| | | 1983, 3322; 1993, 3332; 2042, 3379; 2077, 3412; 2156, 3486; 2165, |
| | | 3495; 2171, 3500; 2178, 3507; 2180, 3509; 2261, 3589; 2283, 3610; |
| | | 2284, 3611; 2329, 3655; 2345, 3671; 2361, 3686; 2403, 3724; |
| | | 2411, 3730; 2430, 3743; 2450, 3762; 2480, 3792; 2031, No_Pept; 2429, |
| | | No_Pept; |
| 42 | 969 | 1416, 2777; 1420, 2781; 1478, 2834; 1612, 2966; 1956, 3300; 2368, |
| | | 3693; 2408, 3729; |
| 43 | 236 | 1564, 2919; 1635, 2989; 1703, 3054; 1769, 3119; 1926, 3271; 2319, |
| | | 3645; 2470, 3782; |
| 44 | 388 | 1313, 2681; 1366, 2730; 1635, 2989; 1769, 3119; 1862, 3210; 1926, |
| | | 3271; 2470, 3782; |
| 45 | 1066 | 1386, 2749; 1662, 3015; 1690, 3042; 1940, 3285; 2132, 3463; 2139, |
| | | 3470; 2249, 3577; |
| 46 | 309 | 1128, 2497; 1147, 2516; 1289, 2657; 1311, 2679; 1314, 2682; 1316, |
| | | 2684; 1338, 2704; 1415, 2776; 1416, 2777; 1456, 2816; 1488, 2844; |
| | | 1498, 2854; 1547, 2902; 1570, 2925; 1574, 2929; 1589, 2944; 1590, |
| | | 2945; 1623, 2977; 1647, 3000; 1655, 3008; 1697, 3049; 1717, 3068; |
| | | 1734, 3085; 1843, 3191; 1867, 3215; 1920, 3265; 2075, 3410; |
| | | 2091, 3426; 2092, 3427; 2094, 3429; 2107, 3440; 2123, 3454; 2127, |
| | | 3458; 2175, 3504; 2190, 3519; 2223, 3551; 2321, 3647; 2447, 3760; |
| 47 | 990 | 1731, 3082; 1912, 3257; |
| 48 | 911 | 1168, 2537; 1731, 3082; 1748, 3098; 1912, 3257; |
| 49 | 307 | 1159, 2528; 1360, 2724; 2350, 3675; |
| 50 | 838 | 1939, 3284; 2131, 3462; |
| 51 | 424 | 1248, 2617; 1407, 2769; 1744, 3094; 1782, 3132; |
| 52 | 760 | 1484, 2840; 1901, 3246; 2201, 3530; 2483, 3795; |
| 53 | 271 | 1185, 2554; 1329, 2695; 1381, 2744; 1425, 2786; 1437, 2798; 1451, |
| | | 2811; 1494, 2850; 1503, 2859; 1554, 2909; 1718, 3069; 1903, 3248; |
| | | 1921, 3266; 1958, 3302; 2023, 3361; 2067, 3402; 2113, 3444; 2126, |
| | | 3457; 2130, 3461; 2222, 3550; |
| 54 | 299 | 1129, 2498; 1223, 2592; 1280, 2648; 1404, 2766; 1443, 2804; 1484, |
| | | 2840; 1625, 2979; 1650, 3003; 1674, 3026; 1715, 3066; 1801, 3150; |
| | | 1950, 3294; 1951, 3295; 2144, 3475; 2185, 3514; 2198, 3527; 2296, |
| | | 3622; 2336, 3662; 2365, 3690; 2366, 3691; 2390, 3712; 2402, 3723; |
| | | 2435, 3748; 2459, 3771; |
| 55 | 418 | 2273, 3601; 1401, No_Pept; |
| 56 | 469 | 1315, 2683; 2222, 3550; |
| 57 | 220, 726, 825 | 1443, 2804; |
| 58 | 465 | 1268, 2637; 1533, 2888; 1616, 2970; |
| 59 | 827 | 1392, 2755; 1585, 2940; 1673, 3025; 2002, 3340; |
| 60 | 755 | 1248, 2617; |
| 61 | 976 | 1248, 2617; |
| 62 | 460 | 1192, 2561; 1215, 2584; 1731, 3082; 1989, 3328; 1959, No_Pept; 2208, |
| | | No_Pept; |
| 63 | 206, 633 | 1303, 2671; 1362, 2726; 1406, 2768; 1515, 2870; 1653, 3006; 2013, |
| | | 3351; 2220, 3548; 2251, 3579; 2381, 3703; 2395, 3717; 2064, No_Pept; |
| | | 2146, No_Pept; 2487, No_Pept; |
| 64 | 1003 | 1132, 2501; 1149, 2518; 1222, 2591; 1343, 2707; 1353, 2717; 1579, |
| | | 2934; 1640, 2994; 1686, 3038; 1745, 3095; 1819, 3168; 1844, 3192; |
| | | 1847, 3195; 1868, 3216; 1902, 3247; 1923, 3268; 1938, 3283; 2348, |
| | | 3674; 2355, 3680; 2377, 3701; |
| 65 | 1070 | 1188, 2557; 1381, 2744; 1414, 2775; 1503, 2859; 2222, 3550; |
| 66 | 618 | 1657, 3010; |
| 67 | 2, 174, 665, 715, 729, 160, 000 | 2089, 3424; 2311, 3637; 2368, 3693; |
| 68 | 52, 581, 910, 591, 063 | 2240, 3568; |
| 69 | 8, 821, 005 | |
| 70 | 4, 154, 177, 281, 068 | 1261, 2630; 1516, 2871; 2347, 3673; 2466, 3778; |
| 71 | 268, 269, 270, 416, 828, 829, 967, 968 | 1261, 2630; 1516, 2871; 2347, 3673; 2466, 3778; |
| 72 | 834 | 1153, 2522; 1365, 2729; 1974, 3315; 2230, 3558; |
| 73 | 630, 833 | 1153, 2522; 1365, 2729; 1974, 3315; 2230, 3558; |
| 74 | 412 | 1153, 2522; 1365, 2729; 2230, 3558; 1645, No_Pept; |
| 75 | 1052 | 1153, 2522; 1365, 2729; 1974, 3315; 2086, 3421; 2113, 3444; 2230, |
| | | 3558; 1645, No_Pept; |
| 76 | 720, 816 | 1205, 2574; 1221, 2590; 1628, 2982; 1661, 3014; |
| 77 | 1018 | 1349, 2713; 1366, 2730; 1635, 2989; 1769, 3119; 1862, 3210; 2176, |
| | | 3505; 2272, 3600; 2319, 3645; 2406, 3727; 2470, 3782; |
| 78 | 996 | 1229, 2598; 1283, 2651; 1418, 2779; 1525, 2880; 1597, 2952; 1764, |
| | | 3114; 1863, 3211; 1905, 3250; 2121, 3452; 2141, 3472; 2151, 3481; |
| | | 2278, 3606; 2444, 3757; 2457, 3769; 2463, 3775; 2472, 3784; |
| 79 | 568 | 2382, 3704; |
| 80 | 913 | 1402, 2764; 1462, 2822; 1946, 3291; 1949, 3293; 2191, 3520; 2475, |
| | | 3787; |
| 81 | 912 | 1645, No_Pept; |
| 82 | 413 | 1365, 2729; |
| 83 | 629 | 1335, 2701; 1855, 3203; 2149, 3479; 2221, 3549; 1645, No_Pept; |
| 84 | 539 | 1386, 2749; 1469, 2829; 1535, 2890; 1662, 3015; 1690, 3042; 1940, |
| | | 3285; 2085, 3420; 2132, 3463; 2249, 3577; |
| 85 | 306, 667, 770, 884 | 1162, 2531; 1225, 2594; 1241, 2610; 1287, 2655; 1308, 2676; 1534, |
| | | 2889; 1691, 3043; 1694, 3046; 1724, 3075; 1838, 3186; 1860, 3208; |
| | | 1866, 3214; 1951, 3295; 2007, 3345; 2045, 3382; 2058, 3394; 2129, |
| | | 3460; 2137, 3468; 2140, 3471; 2199, 3528; 2207, 3536; 2271, 3599; |
| | | 2437, 3750; 2464, 3776; 2467, 3779; 2473, 3785; 2399, No_Pept; |
| 86 | 470 | 1162, 2531; 1225, 2594; 1287, 2655; 1308, 2676; 1325, 2691; 1534, |
| | | 2889; 1691, 3043; 1694, 3046; 1724, 3075; 1798, 3147; 1838, 3186; |
| | | 1860, 3208; 1866, 3214; 1951, 3295; 2007, 3345; 2045, 3382; 2058, |
| | | 3394; 2129, 3460; 2137, 3468; 2140, 3471; 2199, 3528; 2200, 3529; |
| | | 2207, 3536; 2249, 3577; 2271, 3599; 2437, 3750; 2464, 3776; |
| | | 2467, 3779; 2473, 3785; 2399, No_Pept; |
| 87 | 887 | 1821, 3170; 1857, 3205; 2362, 3687; 2491, 3801; |
| 88 | 632 | 1441, 2802; 1720, 3071; 1885, 3233; 2058, 3394; |
| 89 | 817 | 1322, 2688; 1736, 3087; |
| 90 | 722 | |
| 91 | 215 | 2323, 3649; |
| 92 | 734 | 1259, 2628; 1327, 2693; 1354, 2718; 1513, 2868; 1803, 3152; 2285, |
| | | 3612; 2303, 3629; |
| 93 | 631 | 1606, 2960; 1752, 3102; |
| 94 | 858 | 1265, 2634; 1270, 2639; 1498, 2854; 1499, 2855; 1790, 3140; 1867, |
| | | 3215; 1925, 3270; 1944, 3289; 1997, 3336; 2101, 3434; 2167, 3497; |
| | | 2303, 3629; 2310, 3636; 2328, 3654; 2436, 3749; 2168, No_Pept; |
| 95 | 831 | 1537, 2892; 1960, 3303; 2270, 3598; |
| 96 | 290, 296, 297, 461, 462, 463, 464, 570, 658, 762, | |
| | 763, 914, 1055 | |
| 97 | 1118 | 1662, 3015; 1672, 3024; 1758, 3108; 1780, 3130; 1810, 3159; 1832, |
| | | 3180; 1834, 3182; 1837, 3185; 1892, 3239; 1916, 3261; 1999, 3338; |
| | | 2004, 3342; 2102, 3435; 2106, 3439; 2197, 3526; 2434, 3747; |
| 98 | 459 | |
| 99 | 293, 759 | 2177, 3506; 2291, 3618; 2309, 3635; |
| 100 | 961 | 1348, 2712; 2420, 3737; 2482, 3794; |
| 101 | 1008 | 1200, 2569; 1388, 2751; 1501, 2857; 1848, 3196; 1932, 3277; 2065, |
| | | 3400; 2189, 3518; 2226, 3554; 2239, 3567; 2360, 3685; |
| 102 | 737 | 1174, 2543; 1296, 2664; 1317, 2685; 1650, 3003; 1967, 3310; 2394, |
| | | 3716; 2476, 3788; |
| 103 | 204, 205, 986, 987 | 1203, 2572; 1248, 2617; 1660, 3013; 1822, 3171; 2142, 3473; |
| 104 | 452 | 1203, 2572; 1248, 2617; 1660, 3013; 1676, 3028; 1822, 3171; 2142, |
| | | 3473; 2184, 3513; |
| 105 | 1117 | 1133, 2502; 1220, 2589; 1233, 2602; 1240, 2609; 1244, 2613; 1291, |
| | | 2659; 1305, 2673; 1368, 2732; 1372, 2736; 1386, 2749; 1449, 2809; |
| | | 1500, 2856; 1510, 2865; 1512, 2867; 1521, 2876; 1529, 2884; 1543, |
| | | 2898; 1565, 2920; 1613, 2967; 1646, 2999; 1659, 3012; 1708, 3059; |
| | | 1727, 3078; 1733, 3084; 1740, 3090; 1750, 3100; 1767, 3117; |
| | | 1781, 3131; 1789, 3139; 1825, 3174; 1839, 3187; 1859, 3207; 1863, |
| | | 3211; 1891, 3238; 1893, 3240; 1897, 3242; 1927, 3272; 1936, 3281; |
| | | 1970, 3311; 1985, 3324; 2012, 3350; 2018, 3356; 2025, 3363; 2054, |
| | | 3390; 2056, 3392; 2059, 3395; 2063, 3399; 2067, 3402; 2081, 3416; |
| | | 2102, 3435; 2196, 3525; 2211, 3539; 2244, 3572; 2251, 3579; |
| | | 2254, 3582; 2268, 3596; 2281, 3608; 2289, 3616; 2297, 3623; 2308, |
| | | 3634; 2337, 3663; 2357, 3682; 2367, 3692; 2383, 3705; 2387, 3709; |
| | | 2426, 3740; 2458, 3770; 2461, 3773; 2473, 3785; 2478, 3790; |
| 106 | 1113 | 1134, 2503; 1142, 2511; 1165, 2534; 1172, 2541; 1177, 2546; 1184, |
| | | 2553; 1196, 2565; 1214, 2583; 1224, 2593; 1238, 2607; 1267, 2636; |
| | | 1279, 2647; 1309, 2677; 1326, 2692; 1339, 2705; 1360, 2724; 1390, |
| | | 2753; 1460, 2820; 1462, 2822; 1470, 2830; 1484, 2840; 1509, 2864; |
| | | 1523, 2878; 1528, 2883; 1560, 2915; 1572, 2927; 1591, 2946; |
| | | 1600, 2955; 1601, 2956; 1609, 2963; 1615, 2969; 1624, 2978; 1641, |
| | | 2995; 1642, 2996; 1664, 3017; 1677, 3029; 1683, 3035; 1684, 3036; |
| | | 1687, 3039; 1694, 3046; 1706, 3057; 1749, 3099; 1760, 3110; 1775, |
| | | 3125; 1777, 3127; 1788, 3138; 1793, 3143; 1800, 3149; 1801, 3150; |
| | | 1804, 3153; 1884, 3232; 1888, 3235; 1915, 3260; 1970, 3311; |
| | | 1971, 3312; 1991, 3330; 1996, 3335; 2033, 3370; 2043, 3380; 2061, |
| | | 3397; 2062, 3398; 2067, 3402; 2114, 3445; 2119, 3450; 2122, 3453; |
| | | 2164, 3494; 2209, 3537; 2237, 3565; 2257, 3585; 2288, 3615; 2315, |
| | | 3641; 2342, 3668; 2358, 3683; 2370, 3695; 2376, 3700; 1321, No_Pept; |
| | | 1341, No_Pept; 1448, No_Pept; 1474, No_Pept; 1476, No_Pept; |
| | | 1477, No_Pept; 1508, No_Pept; 1605, No_Pept; 1702, No_Pept; |
| | | 1830, No_Pept; 1887, No_Pept; 1895, No_Pept; 1948, No_Pept; |
| | | 1968, No_Pept; 1976, No_Pept; 1978, No_Pept; 2098, No_Pept; |
| | | 2109, No_Pept; 2280, No_Pept; 2293, No_Pept; 2379, No_Pept; |
| | | 2415, No_Pept; 2425, No_Pept; 2449, No_Pept; |
| 107 | 1, 110, 111, 111, 121, 110 | 1135, 2504; 1142, 2511; 1153, 2522; 1157, 2526; 1171, 2540; 1172, |
| | | 2541; 1177, 2546; 1178, 2547; 1204, 2573; 1214, 2583; 1218, 2587; |
| | | 1224, 2593; 1236, 2605; 1237, 2606; 1238, 2607; 1242, 2611; 1250, |
| | | 2619; 1251, 2620; 1262, 2631; 1267, 2636; 1297, 2665; 1300, 2668; |
| | | 1306, 2674; 1339, 2705; 1347, 2711; 1383, 2746; 1390, 2753; |
| | | 1399, 2762; 1411, 2773; 1422, 2783; 1426, 2787; 1433, 2794; 1436, |
| | | 2797; 1442, 2803; 1454, 2814; 1460, 2820; 1462, 2822; 1470, 2830; |
| | | 1480, 2836; 1484, 2840; 1485, 2841; 1496, 2852; 1509, 2864; 1523, |
| | | 2878; 1527, 2882; 1528, 2883; 1552, 2907; 1560, 2915; 1568, 2923; |
| | | 1570, 2925; 1573, 2928; 1576, 2931; 1591, 2946; 1592, 2947; |
| | | 1596, 2951; 1598, 2953; 1599, 2954; 1600, 2955; 1601, 2956; 1609, |
| | | 2963; 1615, 2969; 1622, 2976; 1624, 2978; 1627, 2981; 1632, 2986; |
| | | 1634, 2988; 1642, 2996; 1664, 3017; 1668, 3020; 1677, 3029; 1683, |
| | | 3035; 1684, 3036; 1687, 3039; 1694, 3046; 1700, 3052; 1709, 3060; |
| | | 1711, 3062; 1714, 3065; 1723, 3074; 1749, 3099; 1754, 3104; |
| | | 1775, 3125; 1777, 3127; 1788, 3138; 1800, 3149; 1804, 3153; 1807, |
| | | 3156; 1808, 3157; 1820, 3169; 1878, 3226; 1880, 3228; 1888, 3235; |
| | | 1970, 3311; 1980, 3319; 1982, 3321; 1984, 3323; 1991, 3330; 1992, |
| | | 3331; 1995, 3334; 1996, 3335; 2016, 3354; 2030, 3368; 2033, 3370; |
| | | 2043, 3380; 2055, 3391; 2057, 3393; 2062, 3398; 2087, 3422; |
| | | 2114, 3445; 2119, 3450; 2122, 3453; 2124, 3455; 2128, 3459; 2133, |
| | | 3464; 2143, 3474; 2164, 3494; 2182, 3511; 2186, 3515; 2209, 3537; |
| | | 2210, 3538; 2219, 3547; 2231, 3559; 2237, 3565; 2250, 3578; 2257, |
| | | 3585; 2279, 3607; 2288, 3615; 2292, 3619; 2304, 3630; 2315, 3641; |
| | | 2317, 3643; 2333, 3659; 2340, 3666; 2342, 3668; 2354, 3679; |
| | | 2358, 3683; 2369, 3694; 2386, 3708; 2393, 3715; 2407, 3728; 2414, |
| | | 3733; 2422, 3739; 2455, 3767; 2460, 3772; 2474, 3786; 2477, 3789; |
| | | 1321, No_Pept; 1341, No_Pept; 1448, No_Pept; 1474, No_Pept; |
| | | 1476, No_Pept; 1477, No_Pept; 1508, No_Pept; 1605, No_Pept; |
| | | 1702, No_Pept; 1830, No_Pept; 1887, No_Pept; 1895, No_Pept; 1948, |
| | | No_Pept; 1968, No_Pept; 1976, No_Pept; 1978, No_Pept; 2098, |
| | | No_Pept; 2109, No_Pept; 2280, No_Pept; 2293, No_Pept; 2379, |
| | | No_Pept; 2415, No_Pept; 2425, No_Pept; 2449, No_Pept; |
| 108 | 1115 | 1135, 2504; 1142, 2511; 1153, 2522; 1157, 2526; 1175, 2544; 1214, |
| | | 2583; 1237, 2606; 1238, 2607; 1242, 2611; 1245, 2614; 1250, 2619; |
| | | 1251, 2620; 1262, 2631; 1267, 2636; 1279, 2647; 1294, 2662; 1297, |
| | | 2665; 1300, 2668; 1307, 2675; 1309, 2677; 1328, 2694; 1347, 2711; |
| | | 1390, 2753; 1399, 2762; 1419, 2780; 1422, 2783; 1433, 2794; |
| | | 1436, 2797; 1453, 2813; 1454, 2814; 1460, 2820; 1462, 2822; 1480, |
| | | 2836; 1483, 2839; 1484, 2840; 1496, 2852; 1509, 2864; 1523, 2878; |
| | | 1527, 2882; 1528, 2883; 1543, 2898; 1551, 2906; 1552, 2907; 1560, |
| | | 2915; 1571, 2926; 1576, 2931; 1577, 2932; 1591, 2946; 1596, 2951; |
| | | 1598, 2953; 1600, 2955; 1601, 2956; 1608, 2962; 1609, 2963; |
| | | 1610, 2964; 1624, 2978; 1642, 2996; 1644, 2998; 1658, 3011; 1664, |
| | | 3017; 1666, 3019; 1668, 3020; 1678, 3030; 1683, 3035; 1684, 3036; |
| | | 1687, 3039; 1694, 3046; 1700, 3052; 1709, 3060; 1738, 3089; 1749, |
| | | 3099; 1757, 3107; 1775, 3125; 1776, 3126; 1777, 3127; 1785, 3135; |
| | | 1788, 3138; 1800, 3149; 1807, 3156; 1820, 3169; 1861, 3209; |
| | | 1880, 3228; 1888, 3235; 1913, 3258; 1970, 3311; 1982, 3321; 1984, |
| | | 3323; 1992, 3331; 1994, 3333; 1995, 3334; 1996, 3335; 2011, 3349; |
| | | 2033, 3370; 2048, 3385; 2055, 3391; 2057, 3393; 2096, 3431; 2114, |
| | | 3445; 2119, 3450; 2122, 3453; 2133, 3464; 2164, 3494; 2181, 3510; |
| | | 2182, 3511; 2209, 3537; 2210, 3538; 2218, 3546; 2237, 3565; |
| | | 2250, 3578; 2257, 3585; 2279, 3607; 2287, 3614; 2288, 3615; 2304, |
| | | 3630; 2317, 3643; 2323, 3649; 2333, 3659; 2340, 3666; 2342, 3668; |
| | | 2354, 3679; 2358, 3683; 2359, 3684; 2386, 3708; 2394, 3716; 2404, |
| | | 3725; 2451, 3763; 2455, 3767; 2460, 3772; 2474, 3786; 1321, No_Pept; |
| | | 1341, No_Pept; 1373, No_Pept; 1448, No_Pept; 1474, No_Pept; |
| | | 1476, No_Pept; 1477, No_Pept; 1508, No_Pept; 1605, No_Pept; |
| | | 1702, No_Pept; 1830, No_Pept; 1887, No_Pept; 1895, No_Pept; |
| | | 1948, No_Pept; 1968, No_Pept; 1976, No_Pept; 1978, No_Pept; |
| | | 2098, No_Pept; 2109, No_Pept; 2280, No_Pept; 2293, No_Pept; |
| | | 2379, No_Pept; 2415, No_Pept; 2425, No_Pept; 2449, No_Pept; |
| 109 | 1114 | 1134, 2503; 1135, 2504; 1137, 2506; 1141, 2510; 1142, 2511; 1153, |
| | | 2522; 1157, 2526; 1158, 2527; 1172, 2541; 1178, 2547; 1182, 2551; |
| | | 1190, 2559; 1195, 2564; 1228, 2597; 1236, 2605; 1237, 2606; 1238, |
| | | 2607; 1243, 2612; 1247, 2616; 1251, 2620; 1262, 2631; 1272, 2641; |
| | | 1275, 2644; 1281, 2649; 1294, 2662; 1297, 2665; 1299, 2667; |
| | | 1309, 2677; 1314, 2682; 1326, 2692; 1327, 2693; 1331, 2697; 1332, |
| | | 2698; 1347, 2711; 1351, 2715; 1363, 2727; 1390, 2753; 1397, 2760; |
| | | 1398, 2761; 1410, 2772; 1411, 2773; 1419, 2780; 1424, 2785; 1426, |
| | | 2787; 1433, 2794; 1436, 2797; 1453, 2813; 1454, 2814; 1456, 2816; |
| | | 1460, 2820; 1462, 2822; 1480, 2836; 1483, 2839; 1484, 2840; |
| | | 1490, 2846; 1494, 2850; 1496, 2852; 1504, 2860; 1509, 2864; 1520, |
| | | 2875; 1528, 2883; 1530, 2885; 1538, 2893; 1543, 2898; 1545, 2900; |
| | | 1549, 2904; 1553, 2908; 1562, 2917; 1566, 2921; 1576, 2931; 1582, |
| | | 2937; 1591, 2946; 1592, 2947; 1598, 2953; 1600, 2955; 1601, 2956; |
| | | 1608, 2962; 1609, 2963; 1619, 2973; 1622, 2976; 1627, 2981; |
| | | 1632, 2986; 1642, 2996; 1644, 2998; 1646, 2999; 1654, 3007; 1664, |
| | | 3017; 1683, 3035; 1684, 3036; 1687, 3039; 1696, 3048; 1700, 3052; |
| | | 1701, 3053; 1709, 3060; 1711, 3062; 1712, 3063; 1713, 3064; 1719, |
| | | 3070; 1723, 3074; 1730, 3081; 1749, 3099; 1754, 3104; 1757, 3107; |
| | | 1767, 3117; 1777, 3127; 1779, 3129; 1783, 3133; 1784, 3134; |
| | | 1788, 3138; 1800, 3149; 1801, 3150; 1807, 3156; 1808, 3157; 1809, |
| | | 3158; 1815, 3164; 1823, 3172; 1826, 3175; 1827, 3176; 1836, 3184; |
| | | 1865, 3213; 1869, 3217; 1880, 3228; 1881, 3229; 1882, 3230; 1883, |
| | | 3231; 1884, 3232; 1888, 3235; 1914, 3259; 1915, 3260; 1931, 3276; |
| | | 1935, 3280; 1963, 3306; 1970, 3311; 1979, 3318; 1980, 3319; |
| | | 1981, 3320; 1982, 3321; 1985, 3324; 1996, 3335; 1998, 3337; 2019, |
| | | 3357; 2030, 3368; 2036, 3373; 2043, 3380; 2046, 3383; 2055, 3391; |
| | | 2062, 3398; 2087, 3422; 2089, 3424; 2096, 3431; 2103, 3436; 2114, |
| | | 3445; 2126, 3457; 2133, 3464; 2136, 3467; 2142, 3473; 2143, 3474; |
| | | 2147, 3477; 2161, 3491; 2164, 3494; 2169, 3498; 2206, 3535; |
| | | 2209, 3537; 2210, 3538; 2214, 3542; 2219, 3547; 2231, 3559; 2234, |
| | | 3562; 2235, 3563; 2250, 3578; 2257, 3585; 2287, 3614; 2317, 3643; |
| | | 2325, 3651; 2332, 3658; 2333, 3659; 2335, 3661; 2342, 3668; 2354, |
| | | 3679; 2358, 3683; 2359, 3684; 2364, 3689; 2369, 3694; 2376, 3700; |
| | | 2385, 3707; 2393, 3715; 2398, 3720; 2414, 3733; 2418, 3735; |
| | | 2445, 3758; 2452, 3764; 2455, 3767; 2460, 3772; 2462, 3774; 2474, |
| | | 3786; 2477, 3789; 2485, 3797; 1276, No_Pept; 1320, No_Pept; 1321, |
| | | No_Pept; 1341, No_Pept; 1342, No_Pept; 1448, No_Pept; 1474, |
| | | No_Pept; 1475, No_Pept; 1476, No_Pept; 1477, No_Pept; 1508, |
| | | No_Pept; 1605, No_Pept; 1702, No_Pept; 1830, No_Pept; 1887, |
| | | No_Pept; 1894, No_Pept; 1895, No_Pept; 1948, No_Pept; 1968, No_Pept; |
| | | 1969, No_Pept; 1976, No_Pept; 1978, No_Pept; 2097, No_Pept; |
| | | 2098, No_Pept; 2109, No_Pept; 2110, No_Pept; 2280, No_Pept; |
| | | 2293, No_Pept; 2378, No_Pept; 2379, No_Pept; 2409, No_Pept; |
| | | 2410, No_Pept; 2415, No_Pept; 2416, No_Pept; 2425, No_Pept; |
| | | 2449, No_Pept; |
| 110 | 661 | 1127, 2496; 1519, 2874; 1695, 3047; 1746, 3096; 1922, 3267; 1947, |
| | | 3292; 1972, 3313; 2000, 3339; 2015, 3353; 2072, 3407; 2193, 3522; |
| | | 2374, 3698; |
| 111 | 854 | 1319, 2687; 1405, 2767; 1620, 2974; 1682, 3034; 1707, 3058; 1728, |
| | | 3079; 1746, 3096; 1787, 3137; 1845, 3193; 1945, 3290; 1739, No_Pept; |
| 112 | 771 | 1269, 2638; 1502, 2858; 1575, 2930; 1664, 3017; 1693, 3045; 1829, |
| | | 3178; 1909, 3254; 2040, 3377; 2462, 3774; |
| 113 | 467 | 1162, 2531; 1293, 2661; 1481, 2837; 1517, 2872; 1557, 2912; 1608, |
| | | 2962; 1610, 2964; 1663, 3016; 1670, 3022; 1943, 3288; 1965, 3308; |
| | | 1977, 3317; 1986, 3325; 2041, 3378; 2209, 3537; 2243, 3571; 2284, |
| | | 3611; 2331, 3657; 2469, 3781; |
| 114 | 546 | 1170, 2539; 1173, 2542; 1202, 2571; 1334, 2700; 1382, 2745; 1457, |
| | | 2817; 1464, 2824; 1466, 2826; 1471, 2831; 1472, 2832; 1495, 2851; |
| | | 1541, 2896; 1593, 2948; 1604, 2959; 1649, 3002; 1675, 3027; 1721, |
| | | 3072; 1766, 3116; 1771, 3121; 1774, 3124; 1814, 3163; 1824, 3173; |
| | | 1840, 3188; 1841, 3189; 1852, 3200; 1925, 3270; 1964, 3307; |
| | | 2006, 3344; 2008, 3346; 2076, 3411; 2084, 3419; 2093, 3428; 2111, |
| | | 3442; 2112, 3443; 2142, 3473; 2166, 3496; 2173, 3502; 2179, 3508; |
| | | 2194, 3523; 2217, 3545; 2290, 3617; 2314, 3640; 2339, 3665; 2341, |
| | | 3667; 2343, 3669; 2352, 3677; 2353, 3678; 2428, 3742; 2433, 3746; |
| | | 2442, 3755; 2455, 3767; 2464, 3776; 2466, 3778; |
| 115 | 921 | 1143, 2512; 1153, 2522; 1258, 2627; 1355, 2719; 1371, 2735; 1385, |
| | | 2748; 1417, 2778; 1461, 2821; 1532, 2887; 1638, 2992; 1639, 2993; |
| | | 1743, 3093; 1811, 3160; 1889, 3236; 1898, 3243; 2033, 3370; 2090, |
| | | 3425; 2095, 3430; 2150, 3480; 2216, 3544; 2228, 3556; 2248, 3576; |
| | | 2252, 3580; 2283, 3610; 2400, 3721; 2405, 3726; 2419, 3736; |
| | | 2492, 3802; |
| 116 | 975 | 1129, 2498; 1130, 2499; 1131, 2500; 1136, 2505; 1138, 2507; 1140, |
| | | 2509; 1144, 2513; 1153, 2522; 1156, 2525; 1161, 2530; 1163, 2532; |
| | | 1165, 2534; 1169, 2538; 1186, 2555; 1191, 2560; 1192, 2561; 1197, |
| | | 2566; 1198, 2567; 1204, 2573; 1207, 2576; 1208, 2577; 1209, 2578; |
| | | 1210, 2579; 1227, 2596; 1235, 2604; 1246, 2615; 1255, 2624; |
| | | 1264, 2633; 1282, 2650; 1285, 2653; 1286, 2654; 1290, 2658; 1292, |
| | | 2660; 1295, 2663; 1301, 2669; 1330, 2696; 1333, 2699; 1346, 2710; |
| | | 1350, 2714; 1358, 2722; 1367, 2731; 1370, 2734; 1384, 2747; 1387, |
| | | 2750; 1400, 2763; 1403, 2765; 1408, 2770; 1413, 2774; 1427, 2788; |
| | | 1438, 2799; 1439, 2800; 1440, 2801; 1445, 2806; 1446, 2807; |
| | | 1455, 2815; 1456, 2816; 1458, 2818; 1459, 2819; 1465, 2825; 1482, |
| | | 2838; 1483, 2839; 1511, 2866; 1518, 2873; 1522, 2877; 1524, 2879; |
| | | 1532, 2887; 1533, 2888; 1539, 2894; 1542, 2897; 1559, 2914; 1589, |
| | | 2944; 1602, 2957; 1607, 2961; 1615, 2969; 1626, 2980; 1631, 2985; |
| | | 1636, 2990; 1641, 2995; 1652, 3005; 1663, 3016; 1668, 3020; |
| | | 1669, 3021; 1689, 3041; 1699, 3051; 1704, 3055; 1705, 3056; 1710, |
| | | 3061; 1756, 3106; 1759, 3109; 1762, 3112; 1765, 3115; 1794, 3144; |
| | | 1795, 3145; 1805, 3154; 1806, 3155; 1818, 3167; 1833, 3181; 1842, |
| | | 3190; 1846, 3194; 1847, 3195; 1849, 3197; 1850, 3198; 1871, 3219; |
| | | 1875, 3223; 1877, 3225; 1889, 3236; 1890, 3237; 1899, 3244; |
| | | 1902, 3247; 1911, 3256; 1918, 3263; 1919, 3264; 1929, 3274; 1947, |
| | | 3292; 1954, 3298; 1973, 3314; 2003, 3341; 2014, 3352; 2027, 3365; |
| | | 2028, 3366; 2035, 3372; 2039, 3376; 2044, 3381; 2047, 3384; 2049, |
| | | 3386; 2050, 3387; 2063, 3399; 2071, 3406; 2090, 3425; 2099, 3432; |
| | | 2117, 3448; 2122, 3453; 2145, 3476; 2160, 3490; 2162, 3492; |
| | | 2170, 3499; 2183, 3512; 2192, 3521; 2202, 3531; 2210, 3538; 2213, |
| | | 3541; 2224, 3552; 2225, 3553; 2233, 3561; 2245, 3573; 2255, 3583; |
| | | 2256, 3584; 2268, 3596; 2274, 3602; 2276, 3604; 2291, 3618; 2302, |
| | | 3628; 2307, 3633; 2312, 3638; 2316, 3642; 2320, 3646; 2322, 3648; |
| | | 2327, 3653; 2330, 3656; 2338, 3664; 2346, 3672; 2351, 3676; |
| | | 2363, 3688; 2412, 3731; 2432, 3745; 2443, 3756; 2454, 3766; 2455, |
| | | 3767; 2456, 3768; 2465, 3777; 2489, 3799; 2495, 3804; |
| 117 | 965 | 1154, 2523; 1378, 2741; 1966, 3309; 1990, 3329; 2260, 3588; |
| 118 | 764 | 2423, No_Pept; |
| 119 | 411 | 1155, 2524; 1312, 2680; 1384, 2747; 1428, 2789; 1489, 2845; 1614, |
| | | 2968; 1621, 2975; 1741, 3091; 1755, 3105; 1786, 3136; 1856, 3204; |
| | | 2015, 3353; 2138, 3469; 2172, 3501; 2179, 3508; 2187, 3516; 2205, |
| | | 3534; 2358, 3683; 2439, 3752; 2484, 3796; |
| 120 | 628 | |
| 121 | 832 | 1386, 2749; 1662, 3015; 1940, 3285; 2132, 3463; 2249, 3577; |
| 122 | 213 | 1187, 2556; 1396, 2759; 1434, 2795; 1904, 3249; 1959, No_Pept; 2208, |
| | | No_Pept; |
| 123 | 5, 291, 126 | 1651, 3004; |
| 124 | 530, 531, 532, 533, 534, 535 | 1651, 3004; 2462, 3774; |
| 125 | 211, 292 | |
| 126 | 767 | 1377, 2740; 1441, 2802; 1548, 2903; 1561, 2916; 1594, 2949; 1698, |
| | | 3050; 1747, 3097; 2005, 3343; 2108, 3441; 2238, 3566; 2266, 3594; |
| | | 2318, 3644; 2486, 3798; |
| 127 | 768, 970 | 1130, 2499; 1135, 2504; 1146, 2515; 1160, 2529; 1164, 2533; 1231, |
| | | 2600; 1304, 2672; 1361, 2725; 1374, 2737; 1375, 2738; 1376, 2739; |
| | | 1377, 2740; 1430, 2791; 1450, 2810; 1452, 2812; 1486, 2842; 1567, |
| | | 2922; 1688, 3040; 1725, 3076; 1802, 3151; 1812, 3161; 1859, 3207; |
| | | 1873, 3221; 2015, 3353; 2017, 3355; 2020, 3358; 2024, 3362; |
| | | 2140, 3471; 2152, 3482; 2157, 3487; 2188, 3517; 2237, 3565; 2238, |
| | | 3566; 2295, 3621; 2321, 3647; 2324, 3650; 2371, 3696; 2388, 3710; |
| | | 2389, 3711; 2396, 3718; |
| 128 | 993, 994, 995 | |
| 129 | 207 | |
| 130 | 973 | 1188, 2557; 1206, 2575; 1381, 2744; 1394, 2757; 1500, 2856; 1726, |
| | | 3077; 2154, 3484; 2222, 3550; 2267, 3595; 2384, 3706; |
| 131 | 573 | 1130, 2499; 1139, 2508; 1238, 2607; 1357, 2721; 1358, 2722; 1376, |
| | | 2739; 1536, 2891; 1742, 3092; 1751, 3101; 1761, 3111; 1772, 3122; |
| | | 1797, 3146; 2135, 3466; 2326, 3652; 2417, 3734; |
| 132 | 7, 188, 261, 064 | 2424, No_Pept; |
| 133 | 222 | |
| 134 | 265 | 1249, 2618; |
| 135 | 536 | |
| 136 | 266 | 1189, 2558; 1799, 3148; 1872, 3220; |
| 137 | 974 | 1193, 2562; 1217, 2586; 1393, 2756; 1563, 2918; 1628, 2982; 2277, |
| | | 3605; 2401, 3722; |
| 138 | 254 | 1366, 2730; 1635, 2989; 1769, 3119; 1862, 3210; 2470, 3782; |
| 139 | 636, 637 | 1386, 2749; 1930, 3275; 2249, 3577; 2373, No_Pept; |
| 140 | 422 | 1386, 2749; 1618, 2972; 2032, 3369; 2249, 3577; 2263, 3591; |
| 141 | 989 | |
| 142 | 212 | |
| 143 | 1053 | 2034, 3371; |
| 144 | 634 | 2120, 3451; |
| 145 | 576 | 1192, 2561; 1215, 2584; 1603, 2958; 1763, 3113; 1989, 3328; 1959, |
| | | No_Pept; 2208, No_Pept; |
| 146 | 758 | 2423, No_Pept; 2424, No_Pept; |
| 147 | 721 | |
| 148 | 666 | 1176, 2545; 1254, 2623; 1257, 2626; 1479, 2835; 1722, 3073; 1766, |
| | | 3116; 1791, 3141; 1962, 3305; 2060, 3396; 2115, 3446; 2212, 3540; |
| | | 2305, 3631; 2314, 3640; 2413, 3732; 2479, 3791; |
| 149 | 11, 201, 121 | 1177, 2546; 1226, 2595; 1854, 3202; |
| 150 | 419, 662, 663, 883 | 1177, 2546; 1226, 2595; 1584, 2939; 2241, 3569; |
| 151 | 300, 301, 302, 303, 304 | 1177, 2546; 1226, 2595; 1584, 2939; 2241, 3569; |
| 152 | 836 | 1177, 2546; 1226, 2595; 1369, 2733; 1584, 2939; 1854, 3202; 1870, |
| | | 3218; 1898, 3243; 2241, 3569; 2246, 3574; |
| 153 | 5, 741, 119 | 1177, 2546; 1226, 2595; 1369, 2733; 1584, 2939; 1870, 3218; 1898, |
| | | 3243; 2241, 3569; 2246, 3574; 2262, 3590; 2490, 3800; |
| 154 | 853 | 1153, 2522; 1365, 2729; 1941, 3286; 2086, 3421; 2113, 3444; 2230, |
| | | 3558; 2301, 3627; 1645, No_Pept; |
| 155 | 544, 545, 837 | 1303, 2671; 1362, 2726; 1406, 2768; 1515, 2870; 1653, 3006; 2013, |
| | | 3351; 2220, 3548; 2381, 3703; 2395, 3717; 2064, No_Pept; 2146, |
| | | No_Pept; 2487, No_Pept; |
| 156 | 915 | 1303, 2671; 1362, 2726; 1406, 2768; 1515, 2870; 1653, 3006; 2013, |
| | | 3351; 2184, 3513; 2220, 3548; 2381, 3703; 2395, 3717; 2438, 3751; |
| | | 2064, No_Pept; 2146, No_Pept; 2487, No_Pept; |
| 157 | 761 | 1681, 3033; 2177, 3506; 2291, 3618; |
| 158 | 214 | 1681, 3033; 1908, 3253; 2177, 3506; 2291, 3618; |
| 159 | 2, 951, 054 | 2177, 3506; 2291, 3618; 2309, 3635; |
| 160 | 738 | 1145, 2514; 2184, 3513; |
| 161 | 458 | 1167, 2536; 1216, 2585; 2073, 3408; |
| 162 | 668, 857 | |
| 163 | 1123 | 1497, 2853; 1648, 3001; 1831, 3179; |
| 164 | 1122 | |
| 165 | 7, 651, 069 | 1497, 2853; 1514, 2869; 1583, 2938; 1648, 3001; |
| 166 | 421, 839 | 1180, 2549; 1278, 2646; 1287, 2655; 1308, 2676; 1429, 2790; 1534, |
| | | 2889; 1633, 2987; 1724, 3075; 1816, 3165; 1838, 3186; 1866, 3214; |
| | | 1896, 3241; 2104, 3437; 2129, 3460; 2356, 3681; 2464, 3776; 2399, |
| | | No_Pept; |
| 167 | 736 | 1298, 2666; 1429, 2790; 1534, 2889; 1569, 2924; 1633, 2987; 1685, |
| | | 3037; 1724, 3075; 1816, 3165; 1896, 3241; 2129, 3460; 2249, 3577; |
| | | 2282, 3609; 2464, 3776; 2399, No_Pept; |
| 168 | 922 | 1161, 2530; 1201, 2570; 1253, 2622; 1256, 2625; 1266, 2635; 1282, |
| | | 2650; 1352, 2716; 1359, 2723; 1410, 2772; 1423, 2784; 1470, 2830; |
| | | 1492, 2848; 1493, 2849; 1540, 2895; 1587, 2942; 1595, 2950; 1617, |
| | | 2971; 1637, 2991; 1679, 3031; 1692, 3044; 1735, 3086; 1770, 3120; |
| | | 1792, 3142; 1917, 3262; 1952, 3296; 1957, 3301; 2010, 3348; |
| | | 2053, 3389; 2082, 3417; 2083, 3418; 2125, 3456; 2134, 3465; 2153, |
| | | 3483; 2165, 3495; 2227, 3555; 2313, 3639; 2314, 3640; 2385, 3707; |
| | | 2468, 3780; |
| 169 | 1065 | 1148, 2517; 1225, 2594; 1234, 2603; 1239, 2608; 1284, 2652; 1318, |
| | | 2686; 1357, 2721; 1364, 2728; 1414, 2775; 1435, 2796; 1463, 2823; |
| | | 1506, 2862; 1526, 2881; 1531, 2886; 1558, 2913; 1580, 2935; 1586, |
| | | 2941; 1813, 3162; 1900, 3245; 1937, 3282; 1955, 3299; 2068, 3403; |
| | | 2070, 3405; 2148, 3478; 2163, 3493; 2180, 3509; 2203, 3532; |
| | | 2204, 3533; 2242, 3570; 2380, 3702; 2392, 3714; 2441, 3754; 2455, |
| | | 3767; |
| 170 | 8, 851, 007 | 1199, 2568; 1232, 2601; 1337, 2703; 1467, 2827; 1715, 3066; 1988, |
| | | 3327; 2037, 3374; 2051, 3388; 2300, 3626; 2306, 3632; 2429, No_Pept; |
| 171 | 522 | 1141, 2510; 1181, 2550; 1184, 2553; 1190, 2559; 1219, 2588; 1263, |
| | | 2632; 1345, 2709; 1389, 2752; 1403, 2765; 1451, 2811; 1467, 2827; |
| | | 1468, 2828; 1491, 2847; 1546, 2901; 1665, 3018; 1874, 3222; 2037, |
| | | 3374; 2051, 3388; 2078, 3413; 2080, 3415; 2136, 3467; 2159, 3489; |
| | | 2174, 3503; 2215, 3543; 2229, 3557; 2232, 3560; 2306, 3632; |
| | | 2368, 3693; 2440, 3753; 2031, No_Pept; 2429, No_Pept; |
| 172 | 889 | 1141, 2510; 1181, 2550; 1184, 2553; 1190, 2559; 1194, 2563; 1219, |
| | | 2588; 1263, 2632; 1271, 2640; 1323, 2689; 1345, 2709; 1389, 2752; |
| | | 1392, 2755; 1403, 2765; 1451, 2811; 1467, 2827; 1468, 2828; 1491, |
| | | 2847; 1544, 2899; 1546, 2901; 1665, 3018; 1671, 3023; 1817, 3166; |
| | | 1874, 3222; 1988, 3327; 2021, 3359; 2022, 3360; 2037, 3374; |
| | | 2051, 3388; 2078, 3413; 2080, 3415; 2136, 3467; 2159, 3489; 2174, |
| | | 3503; 2215, 3543; 2229, 3557; 2232, 3560; 2306, 3632; 2368, 3693; |
| | | 2391, 3713; 2440, 3753; 2031, No_Pept; 2429, No_Pept; |
| 173 | 1067 | 1275, 2644; 1933, 3278; 2247, 3575; 2286, 3613; |
| 174 | 962 | 1275, 2644; 1409, 2771; 1421, 2782; 1431, 2792; 1432, 2793; 1933, |
| | | 3278; 2069, 3404; |
| 175 | 963, 964 | 1275, 2644; 1409, 2771; 1431, 2792; 1432, 2793; 1933, 3278; 2069, |
| | | 3404; 2431, 3744; |
| 176 | 972 | 2222, 3550; 2264, 3592; |
| 177 | 575, 971 | 2066, 3401; 2222, 3550; 2264, 3592; 2481, 3793; |
| 178 | 305, 769 | 2264, 3592; |
| 179 | 541 | 1252, 2621; 1302, 2670; 1444, 2805; 1578, 2933; 1729, 3080; 1886, |
| | | 3234; 1906, 3251; 2029, 3367; 2088, 3423; 2195, 3524; 2258, 3586; |
| | | 2259, 3587; 2453, 3765; |
| 180 | 294 | |
| 181 | 1006 | |
| 182 | 818 | 1737, 3088; |
| 183 | 272, 664 | |
| 184 | 216 | 1505, 2861; 1629, 2983; 1753, 3103; 1768, 3118; 2116, 3447; 2298, |
| | | 3624; 2300, 3626; 2344, 3670; 2424, No_Pept; |
| 185 | 1125 | 1858, 3206; |
| 186 | 543 | 1152, 2521; 1483, 2839; 1507, 2863; 2038, 3375; 2118, 3449; 2446, |
| | | 3759; |
| 187 | 1124 | 1483, 2839; 1656, 3009; 1851, 3199; 1858, 3206; 2118, 3449; |
| 188 | 210 | 1274, 2643; 1483, 2839; 2038, 3375; 2118, 3449; 2238, 3566; |
| 189 | 998 | 1310, 2678; 1555, 2910; 1556, 2911; 1581, 2936; 1657, 3010; 2421, |
| | | 3738; |
| 190 | 6, 277, 308, 301, 004 | 1872, 3220; 1953, 3297; 1961, 3304; 2155, 3485; |
| 191 | 991, 992 | 1973, 3314; |
| 192 | 1104 | |
| 193 | 1071 | 1230, 2599; 1657, 3010; 1864, 3212; |
| 194 | 619 | 1230, 2599; 1657, 3010; |
| 195 | 999 | 1230, 2599; 1657, 3010; |
| 196 | 468 | |
| 197 | 766 | |

TABLE 2
Trait values for microRNA targets and associated traits

| Target | Target | | | | |
|---|---|---|---|---|---|
| Gene | Gene | | | Relative | |
| DNA | Peptide | | Relative | Nitro- | Relative |
| SEQ | SEQ ID | Relevant Traits for | Drought | gen | Yield |
| ID No: | No: | miRNA Targets | Value | Value | Value |
| 1128 | 2497 | Drought-Nitrogen-Yield | 0.745 | 1.000 | 1.000 |
| 1130 | 2499 | Drought-Nitrogen-Yield | 0.745 | 1.000 | 1.000 |
| 1136 | 2505 | Drought-Nitrogen-Yield | 0.780 | 0.517 | 0.757 |
| 1138 | 2507 | Drought-Nitrogen-Yield | 0.555 | 0.654 | 0.979 |
| 1145 | 2514 | Drought-Nitrogen-Yield | 0.786 | 0.762 | 0.877 |
| 1147 | 2516 | Drought-Nitrogen-Yield | 1.000 | 0.763 | 0.784 |
| 1157 | 2526 | Drought-Nitrogen-Yield | 0.549 | 0.647 | 0.950 |
| 1161 | 2530 | Drought-Nitrogen-Yield | 0.923 | 0.621 | 0.678 |
| 1167 | 2536 | Drought-Nitrogen-Yield | 0.513 | 0.606 | 0.710 |
| 1173 | 2542 | Drought-Nitrogen-Yield | 0.688 | 0.830 | 0.716 |
| 1254 | 2623 | Drought-Nitrogen-Yield | 0.919 | 0.991 | 0.844 |
| 1265 | 2634 | Drought-Nitrogen-Yield | 0.726 | 0.554 | 0.651 |
| 1308 | 2676 | Drought-Nitrogen-Yield | 0.614 | 0.538 | 0.843 |
| 1342 | N.A. | Drought-Nitrogen-Yield | 0.481 | 0.609 | 0.699 |
| 1390 | 2753 | Drought-Nitrogen-Yield | 0.544 | 0.804 | 0.713 |
| 1471 | 2831 | Drought-Nitrogen-Yield | 0.522 | 0.591 | 0.668 |
| 1472 | 2832 | Drought-Nitrogen-Yield | 0.522 | 0.591 | 0.668 |
| 1533 | 2888 | Drought-Nitrogen-Yield | 0.504 | 0.618 | 0.678 |
| 1537 | 2892 | Drought-Nitrogen-Yield | 0.502 | 0.688 | 0.653 |
| 1540 | 2895 | Drought-Nitrogen-Yield | 0.502 | 0.618 | 0.773 |
| 1588 | 2943 | Drought-Nitrogen-Yield | 0.485 | 0.609 | 0.720 |
| 1592 | 2947 | Drought-Nitrogen-Yield | 0.483 | 0.609 | 0.699 |
| 1600 | 2955 | Drought-Nitrogen-Yield | 0.481 | 0.609 | 0.740 |
| 1605 | N.A. | Drought-Nitrogen-Yield | 0.481 | 0.609 | 0.699 |
| 1621 | 2975 | Drought-Nitrogen-Yield | 0.477 | 0.779 | 0.755 |
| 1703 | 3054 | Drought-Nitrogen-Yield | 0.461 | 0.541 | 0.659 |
| 1129 | 2498 | Drought-Nitrogen | 0.745 | 0.000 | 1.000 |
| 1132 | 2501 | Drought-Nitrogen | 0.745 | 0.435 | 1.000 |
| 1134 | 2503 | Drought-Nitrogen | 0.593 | 0.582 | 0.507 |
| 1155 | 2524 | Drought-Nitrogen | 0.645 | 0.500 | 0.543 |
| 1199 | 2568 | Drought-Nitrogen | 0.466 | 0.580 | 0.615 |
| 1233 | 2602 | Drought-Nitrogen | 0.548 | 0.676 | 0.614 |
| 1237 | 2606 | Drought-Nitrogen | 0.485 | 0.612 | 0.631 |
| 1244 | 2613 | Drought-Nitrogen | 0.546 | 0.615 | 0.534 |
| 1249 | 2618 | Drought-Nitrogen | 0.462 | 0.600 | 0.582 |
| 1260 | 2629 | Drought-Nitrogen | 0.810 | 0.545 | 0.594 |
| 1263 | 2632 | Drought-Nitrogen | 0.736 | 0.490 | 0.489 |
| 1271 | 2640 | Drought-Nitrogen | 0.701 | 0.499 | 0.515 |
| 1284 | 2652 | Drought-Nitrogen | 0.652 | 0.550 | 0.549 |
| 1292 | 2660 | Drought-Nitrogen | 0.639 | 0.599 | 0.576 |
| 1296 | 2664 | Drought-Nitrogen | 0.631 | 0.506 | 0.594 |
| 1317 | 2685 | Drought-Nitrogen | 0.601 | 0.771 | 0.350 |
| 1329 | 2695 | Drought-Nitrogen | 0.586 | 0.589 | 0.528 |
| 1356 | 2720 | Drought-Nitrogen | 0.570 | 0.618 | 0.604 |
| 1379 | 2742 | Drought-Nitrogen | 0.550 | 0.626 | 0.249 |
| 1389 | 2752 | Drought-Nitrogen | 0.545 | 0.618 | 0.631 |
| 1394 | 2757 | Drought-Nitrogen | 0.543 | 0.501 | 0.510 |
| 1396 | 2759 | Drought-Nitrogen | 0.540 | 0.620 | 0.410 |
| 1408 | 2770 | Drought-Nitrogen | 0.535 | 0.507 | 0.533 |
| 1419 | 2780 | Drought-Nitrogen | 0.532 | 0.490 | 0.422 |
| 1429 | 2790 | Drought-Nitrogen | 0.529 | 0.686 | 0.619 |
| 1434 | 2795 | Drought-Nitrogen | 0.528 | 0.601 | 0.000 |
| 1435 | 2796 | Drought-Nitrogen | 0.528 | 0.629 | 0.419 |
| 1436 | 2797 | Drought-Nitrogen | 0.528 | 0.591 | 0.618 |
| 1438 | 2799 | Drought-Nitrogen | 0.528 | 0.498 | 0.587 |
| 1441 | 2802 | Drought-Nitrogen | 0.527 | 0.574 | 0.387 |
| 1451 | 2811 | Drought-Nitrogen | 0.525 | 0.591 | 0.568 |
| 1454 | 2814 | Drought-Nitrogen | 0.523 | 0.723 | 0.583 |
| 1458 | 2818 | Drought-Nitrogen | 0.523 | 0.501 | 0.510 |
| 1459 | 2819 | Drought-Nitrogen | 0.523 | 0.501 | 0.510 |
| 1460 | 2820 | Drought-Nitrogen | 0.523 | 0.591 | 0.618 |
| 1462 | 2822 | Drought-Nitrogen | 0.523 | 0.649 | 0.348 |
| 1473 | 2833 | Drought-Nitrogen | 0.522 | 0.591 | 0.618 |
| 1474 | N.A. | Drought-Nitrogen | 0.522 | 0.591 | 0.568 |
| 1475 | N.A. | Drought-Nitrogen | 0.522 | 0.591 | 0.568 |
| 1476 | N.A. | Drought-Nitrogen | 0.522 | 0.591 | 0.568 |
| 1477 | N.A. | Drought-Nitrogen | 0.522 | 0.591 | 0.568 |
| 1478 | 2834 | Drought-Nitrogen | 0.522 | 0.591 | 0.568 |
| 1479 | 2835 | Drought-Nitrogen | 0.522 | 0.591 | 0.568 |
| 1480 | 2836 | Drought-Nitrogen | 0.522 | 0.591 | 0.568 |
| 1481 | 2837 | Drought-Nitrogen | 0.522 | 0.591 | 0.568 |
| 1486 | 2842 | Drought-Nitrogen | 0.520 | 0.499 | 0.578 |
| 1491 | 2847 | Drought-Nitrogen | 0.518 | 0.516 | 0.620 |
| 1493 | 2849 | Drought-Nitrogen | 0.517 | 0.995 | 0.576 |
| 1501 | 2857 | Drought-Nitrogen | 0.513 | 0.626 | 0.304 |
| 1529 | 2884 | Drought-Nitrogen | 0.505 | 0.609 | 0.630 |
| 1563 | 2918 | Drought-Nitrogen | 0.497 | 0.589 | 0.491 |
| 1581 | 2936 | Drought-Nitrogen | 0.489 | 0.167 | 1.000 |
| 1584 | 2939 | Drought-Nitrogen | 0.488 | 0.612 | 0.630 |
| 1587 | 2942 | Drought-Nitrogen | 0.486 | 0.590 | 0.458 |
| 1593 | 2948 | Drought-Nitrogen | 0.482 | 0.597 | 0.534 |
| 1599 | 2954 | Drought-Nitrogen | 0.481 | 0.517 | 0.432 |
| 1601 | 2956 | Drought-Nitrogen | 0.481 | 0.609 | 0.630 |
| 1602 | 2957 | Drought-Nitrogen | 0.481 | 0.609 | 0.630 |
| 1603 | 2958 | Drought-Nitrogen | 0.481 | 0.609 | 0.630 |
| 1604 | 2959 | Drought-Nitrogen | 0.481 | 0.609 | 0.630 |
| 1611 | 2965 | Drought-Nitrogen | 0.480 | 0.554 | 0.361 |
| 1612 | 2966 | Drought-Nitrogen | 0.480 | 0.554 | 0.361 |
| 1613 | 2967 | Drought-Nitrogen | 0.480 | 0.554 | 0.560 |
| 1629 | 2983 | Drought-Nitrogen | 0.475 | 0.498 | 0.532 |
| 1641 | 2995 | Drought-Nitrogen | 0.472 | 0.541 | 0.604 |
| 1642 | 2996 | Drought-Nitrogen | 0.472 | 0.585 | 0.387 |
| 1683 | 3035 | Drought-Nitrogen | 0.464 | 0.541 | 0.469 |
| 1685 | 3037 | Drought-Nitrogen | 0.464 | 0.801 | 0.354 |
| 1704 | 3055 | Drought-Nitrogen | 0.461 | 0.541 | 0.469 |
| 1707 | 3058 | Drought-Nitrogen | 0.460 | 0.656 | 0.446 |
| 1168 | 2537 | Nitrogen-Yield | 0.305 | 0.548 | 0.705 |
| 1178 | 2547 | Nitrogen-Yield | 0.354 | 0.500 | 0.841 |
| 1179 | 2548 | Nitrogen-Yield | 0.440 | 0.983 | 0.767 |
| 1185 | 2554 | Nitrogen-Yield | 0.295 | 0.597 | 0.679 |
| 1194 | 2563 | Nitrogen-Yield | 0.357 | 0.500 | 0.683 |
| 1220 | 2589 | Nitrogen-Yield | 0.325 | 0.505 | 0.645 |
| 1710 | 3061 | Nitrogen-Yield | 0.456 | 0.569 | 0.652 |
| 1716 | 3067 | Nitrogen-Yield | 0.452 | 0.668 | 0.649 |
| 1733 | 3084 | Nitrogen-Yield | 0.438 | 0.572 | 0.652 |
| 1738 | 3089 | Nitrogen-Yield | 0.434 | 0.569 | 0.652 |
| 1771 | 3121 | Nitrogen-Yield | 0.415 | 0.580 | 0.662 |
| 1784 | 3134 | Nitrogen-Yield | 0.399 | 0.738 | 0.646 |
| 1795 | 3145 | Nitrogen-Yield | 0.388 | 0.767 | 0.654 |
| 1807 | 3156 | Nitrogen-Yield | 0.385 | 0.813 | 0.691 |
| 1823 | 3172 | Nitrogen-Yield | 0.374 | 0.492 | 0.732 |
| 1872 | 3220 | Nitrogen-Yield | 0.353 | 0.570 | 0.681 |
| 1892 | 3239 | Nitrogen-Yield | 0.345 | 0.536 | 0.771 |
| 1926 | 3271 | Nitrogen-Yield | 0.328 | 0.607 | 0.723 |
| 1936 | 3281 | Nitrogen-Yield | 0.322 | 0.681 | 0.729 |
| 1937 | 3282 | Nitrogen-Yield | 0.322 | 0.501 | 0.670 |
| 1938 | 3283 | Nitrogen-Yield | 0.322 | 0.501 | 0.670 |
| 1942 | 3287 | Nitrogen-Yield | 0.321 | 0.554 | 0.743 |
| 1970 | 3311 | Nitrogen-Yield | 0.306 | 0.528 | 0.668 |
| 2001 | 3287 | Nitrogen-Yield | 0.298 | 0.545 | 0.743 |
| 2003 | 3341 | Nitrogen-Yield | 0.297 | 0.554 | 0.735 |
| 2006 | 3344 | Nitrogen-Yield | 0.296 | 0.530 | 0.694 |
| 2026 | 3364 | Nitrogen-Yield | 0.287 | 0.545 | 0.743 |
| 2074 | 3409 | Nitrogen-Yield | 0.274 | 0.596 | 0.650 |
| 2105 | 3438 | Nitrogen-Yield | 0.259 | 0.593 | 0.656 |
| 2109 | N.A. | Nitrogen-Yield | 0.256 | 0.580 | 0.723 |
| 2110 | N.A. | Nitrogen-Yield | 0.256 | 0.580 | 0.723 |
| 2130 | 3461 | Nitrogen-Yield | 0.244 | 0.833 | 0.800 |
| 2145 | 3476 | Nitrogen-Yield | 0.227 | 0.490 | 0.735 |
| 2152 | 3482 | Nitrogen-Yield | 0.220 | 0.515 | 0.723 |
| 2174 | 3503 | Nitrogen-Yield | 0.204 | 0.692 | 0.692 |
| 2175 | 3504 | Nitrogen-Yield | 0.204 | 0.692 | 0.692 |
| 2189 | 3518 | Nitrogen-Yield | 0.190 | 0.779 | 0.755 |
| 2192 | 3521 | Nitrogen-Yield | 0.188 | 0.704 | 0.701 |
| 2199 | 3528 | Nitrogen-Yield | 0.179 | 0.490 | 0.751 |
| 2200 | 3529 | Nitrogen-Yield | 0.179 | 0.490 | 0.751 |
| 2202 | 3531 | Nitrogen-Yield | 0.176 | 0.911 | 0.659 |
| 2240 | 3568 | Nitrogen-Yield | 0.125 | 0.603 | 0.657 |
| 2245 | 3573 | Nitrogen-Yield | 0.119 | 0.569 | 0.714 |
| 2246 | 3574 | Nitrogen-Yield | 0.119 | 0.569 | 0.779 |
| 2291 | 3618 | Nitrogen-Yield | 0.045 | 0.510 | 0.699 |
| 2299 | 3625 | Nitrogen-Yield | 0.043 | 0.600 | 0.657 |
| 2310 | 3636 | Nitrogen-Yield | 0.013 | 0.496 | 0.789 |
| 2313 | 3639 | Nitrogen-Yield | 0.013 | 0.589 | 0.684 |
| 2340 | 3666 | Nitrogen-Yield | 0.000 | 0.754 | 0.670 |
| 2341 | 3667 | Nitrogen-Yield | 0.000 | 0.754 | 0.670 |
| 2371 | 3696 | Nitrogen-Yield | 0.000 | 0.711 | 0.650 |
| 2412 | 3731 | Nitrogen-Yield | 0.000 | 0.600 | 0.657 |
| 2413 | 3732 | Nitrogen-Yield | 0.000 | 0.600 | 0.657 |
| 2414 | 3733 | Nitrogen-Yield | 0.000 | 0.791 | 0.665 |
| 2417 | 3734 | Nitrogen-Yield | 0.000 | 0.511 | 0.725 |
| 2429 | N.A. | Nitrogen-Yield | 0.000 | 0.688 | 0.645 |
| 2430 | 3743 | Nitrogen-Yield | 0.000 | 0.688 | 0.653 |
| 2431 | 3744 | Nitrogen-Yield | 0.000 | 0.688 | 0.653 |
| 2443 | 3756 | Nitrogen-Yield | 0.000 | 0.779 | 0.755 |
| 2468 | 3780 | Nitrogen-Yield | 0.000 | 0.517 | 0.710 |
| 1135 | 2504 | Drought-Yield | 0.591 | 0.321 | 0.798 |
| 1137 | 2506 | Drought-Yield | 0.566 | 0.353 | 0.891 |
| 1141 | 2510 | Drought-Yield | 0.549 | 0.000 | 0.658 |
| 1142 | 2511 | Drought-Yield | 0.716 | 0.430 | 0.829 |
| 1143 | 2512 | Drought-Yield | 0.661 | 0.000 | 0.924 |
| 1146 | 2515 | Drought-Yield | 0.598 | 0.407 | 0.667 |
| 1153 | 2522 | Drought-Yield | 0.663 | 0.212 | 0.909 |
| 1154 | 2523 | Drought-Yield | 0.674 | 0.183 | 0.686 |
| 1160 | 2529 | Drought-Yield | 0.569 | 0.280 | 0.775 |
| 1164 | 2533 | Drought-Yield | 0.635 | 0.400 | 0.770 |
| 1166 | 2535 | Drought-Yield | 0.470 | 0.299 | 0.656 |
| 1169 | 2538 | Drought-Yield | 0.556 | 0.300 | 0.872 |
| 1183 | 2552 | Drought-Yield | 0.642 | 0.365 | 0.783 |
| 1190 | 2559 | Drought-Yield | 0.544 | 0.212 | 0.813 |
| 1192 | 2561 | Drought-Yield | 0.477 | 0.444 | 0.837 |
| 1195 | 2564 | Drought-Yield | 0.522 | 0.200 | 0.724 |
| 1208 | 2577 | Drought-Yield | 0.555 | 0.319 | 0.812 |
| 1231 | 2600 | Drought-Yield | 0.479 | 0.273 | 0.743 |
| 1255 | 2624 | Drought-Yield | 0.919 | 0.000 | 0.686 |
| 1256 | 2625 | Drought-Yield | 0.919 | 0.407 | 0.688 |
| 1258 | 2627 | Drought-Yield | 0.846 | 0.338 | 0.734 |
| 1267 | 2636 | Drought-Yield | 0.712 | 0.122 | 0.662 |
| 1275 | 2644 | Drought-Yield | 0.693 | 0.000 | 0.689 |
| 1278 | 2646 | Drought-Yield | 0.691 | 0.000 | 0.729 |
| 1279 | 2647 | Drought-Yield | 0.681 | 0.301 | 0.763 |
| 1283 | 2651 | Drought-Yield | 0.652 | 0.167 | 0.725 |
| 1290 | 2658 | Drought-Yield | 0.644 | 0.363 | 0.654 |
| 1299 | 2667 | Drought-Yield | 0.630 | 0.000 | 0.696 |
| 1307 | 2675 | Drought-Yield | 0.617 | 0.401 | 0.656 |
| 1322 | 2688 | Drought-Yield | 0.597 | 0.287 | 0.659 |
| 1336 | 2702 | Drought-Yield | 0.581 | 0.228 | 0.746 |
| 1339 | 2705 | Drought-Yield | 0.579 | 0.255 | 0.675 |
| 1342 | N.A. | Drought-Yield | 0.525 | 0.280 | 0.672 |
| 1347 | 2711 | Drought-Yield | 0.575 | 0.378 | 0.898 |
| 1353 | 2717 | Drought-Yield | 0.572 | 0.000 | 0.750 |
| 1355 | 2719 | Drought-Yield | 0.571 | 0.441 | 0.669 |
| 1361 | 2725 | Drought-Yield | 0.565 | 0.468 | 0.674 |
| 1362 | 2726 | Drought-Yield | 0.564 | 0.359 | 0.883 |
| 1363 | 2727 | Drought-Yield | 0.563 | 0.000 | 0.765 |
| 1373 | N.A. | Drought-Yield | 0.555 | 0.000 | 0.697 |
| 1378 | 2741 | Drought-Yield | 0.550 | 0.347 | 0.776 |
| 1409 | 2771 | Drought-Yield | 0.534 | 0.280 | 0.673 |
| 1415 | 2776 | Drought-Yield | 0.532 | 0.285 | 0.752 |
| 1430 | 2791 | Drought-Yield | 0.529 | 0.320 | 0.672 |
| 1431 | 2792 | Drought-Yield | 0.528 | 0.280 | 0.672 |
| 1432 | 2793 | Drought-Yield | 0.528 | 0.280 | 0.672 |
| 1437 | 2798 | Drought-Yield | 0.528 | 0.416 | 0.769 |
| 1448 | N.A. | Drought-Yield | 0.525 | 0.280 | 0.672 |
| 1449 | 2809 | Drought-Yield | 0.525 | 0.280 | 0.672 |
| 1452 | 2812 | Drought-Yield | 0.525 | 0.301 | 0.706 |
| 1453 | 2813 | Drought-Yield | 0.524 | 0.368 | 0.683 |
| 1468 | 2828 | Drought-Yield | 0.522 | 0.378 | 0.699 |
| 1487 | 2843 | Drought-Yield | 0.520 | 0.301 | 0.706 |
| 1498 | 2854 | Drought-Yield | 0.514 | 0.475 | 0.688 |
| 1505 | 2861 | Drought-Yield | 0.511 | 0.000 | 0.800 |
| 1552 | 2907 | Drought-Yield | 0.500 | 0.281 | 0.697 |
| 1562 | 2917 | Drought-Yield | 0.498 | 0.000 | 0.843 |
| 1575 | 2930 | Drought-Yield | 0.492 | 0.000 | 0.813 |
| 1615 | 2969 | Drought-Yield | 0.479 | 0.278 | 0.723 |
| 1643 | 2997 | Drought-Yield | 0.471 | 0.167 | 0.644 |
| 1655 | 3008 | Drought-Yield | 0.469 | 0.361 | 0.844 |
| 1662 | 3015 | Drought-Yield | 0.468 | 0.200 | 0.692 |
| 1664 | 3017 | Drought-Yield | 0.467 | 0.000 | 0.769 |
| 1680 | 3032 | Drought-Yield | 0.465 | 0.159 | 0.662 |
| 1684 | 3036 | Drought-Yield | 0.464 | 0.180 | 0.715 |
| 1177 | 2546 | Nitrogen | 0.460 | 0.500 | 0.634 |
| 1180 | 2549 | Nitrogen | 0.454 | 0.743 | 0.468 |
| 1198 | 2567 | Nitrogen | 0.279 | 0.505 | 0.607 |
| 1206 | 2575 | Nitrogen | 0.153 | 0.504 | 0.310 |
| 1207 | 2576 | Nitrogen | 0.176 | 0.503 | 0.315 |
| 1216 | 2585 | Nitrogen | 0.410 | 0.983 | 0.318 |
| 1218 | 2587 | Nitrogen | 0.294 | 0.643 | 0.492 |
| 1234 | 2603 | Nitrogen | 0.296 | 0.685 | 0.511 |
| 1246 | 2615 | Nitrogen | 0.349 | 0.523 | 0.467 |
| 1342 | N.A. | Nitrogen | 0.305 | 0.548 | 0.507 |
| 1342 | N.A. | Nitrogen | 0.047 | 0.546 | 0.617 |
| 1342 | N.A. | Nitrogen | 0.045 | 0.529 | 0.456 |
| 1342 | N.A. | Nitrogen | 0.000 | 0.747 | 0.542 |
| 1541 | 2896 | Nitrogen | 0.361 | 0.578 | 0.387 |
| 1711 | 3062 | Nitrogen | 0.454 | 0.561 | 0.578 |
| 1715 | 3066 | Nitrogen | 0.453 | 0.580 | 0.615 |
| 1717 | 3068 | Nitrogen | 0.450 | 0.499 | 0.528 |
| 1720 | 3071 | Nitrogen | 0.448 | 0.501 | 0.491 |
| 1721 | 3072 | Nitrogen | 0.448 | 0.578 | 0.531 |
| 1722 | 3073 | Nitrogen | 0.447 | 0.698 | 0.492 |
| 1726 | 3077 | Nitrogen | 0.441 | 0.586 | 0.479 |
| 1727 | 3078 | Nitrogen | 0.441 | 0.541 | 0.629 |
| 1728 | 3079 | Nitrogen | 0.441 | 0.569 | 0.474 |
| 1731 | 3082 | Nitrogen | 0.439 | 0.741 | 0.595 |
| 1734 | 3085 | Nitrogen | 0.437 | 0.513 | 0.555 |
| 1737 | 3088 | Nitrogen | 0.435 | 0.595 | 0.577 |
| 1739 | N.A. | Nitrogen | 0.434 | 0.569 | 0.474 |
| 1740 | 3090 | Nitrogen | 0.434 | 0.569 | 0.474 |
| 1741 | 3091 | Nitrogen | 0.434 | 0.569 | 0.474 |
| 1742 | 3092 | Nitrogen | 0.432 | 0.540 | 0.383 |
| 1745 | 3095 | Nitrogen | 0.430 | 0.623 | 0.535 |
| 1752 | 3102 | Nitrogen | 0.426 | 0.513 | 0.489 |
| 1753 | 3103 | Nitrogen | 0.425 | 0.710 | 0.469 |
| 1757 | 3107 | Nitrogen | 0.424 | 0.498 | 0.532 |
| 1759 | 3109 | Nitrogen | 0.422 | 0.498 | 0.532 |
| 1760 | 3110 | Nitrogen | 0.422 | 0.498 | 0.468 |
| 1761 | 3111 | Nitrogen | 0.421 | 0.676 | 0.445 |
| 1763 | 3113 | Nitrogen | 0.420 | 0.858 | 0.413 |
| 1764 | 3114 | Nitrogen | 0.419 | 0.498 | 0.555 |
| 1765 | 3115 | Nitrogen | 0.419 | 0.498 | 0.555 |
| 1766 | 3116 | Nitrogen | 0.419 | 0.498 | 0.555 |
| 1767 | 3117 | Nitrogen | 0.419 | 0.498 | 0.555 |
| 1775 | 3125 | Nitrogen | 0.408 | 0.738 | 0.595 |
| 1777 | 3127 | Nitrogen | 0.404 | 0.576 | 0.510 |
| 1778 | 3128 | Nitrogen | 0.404 | 0.576 | 0.510 |
| 1781 | 3131 | Nitrogen | 0.401 | 0.580 | 0.321 |
| 1783 | 3133 | Nitrogen | 0.399 | 0.738 | 0.595 |
| 1785 | 3135 | Nitrogen | 0.399 | 0.508 | 0.411 |
| 1786 | 3136 | Nitrogen | 0.398 | 0.504 | 0.514 |
| 1808 | 3157 | Nitrogen | 0.384 | 0.593 | 0.540 |
| 1809 | 3158 | Nitrogen | 0.384 | 0.768 | 0.304 |
| 1810 | 3159 | Nitrogen | 0.384 | 0.673 | 0.539 |
| 1812 | 3161 | Nitrogen | 0.381 | 0.706 | 0.380 |
| 1813 | 3162 | Nitrogen | 0.379 | 0.764 | 0.304 |
| 1814 | 3163 | Nitrogen | 0.378 | 0.556 | 0.491 |
| 1815 | 3164 | Nitrogen | 0.378 | 0.511 | 0.551 |
| 1820 | 3169 | Nitrogen | 0.375 | 0.677 | 0.447 |
| 1825 | 3174 | Nitrogen | 0.373 | 0.545 | 0.456 |
| 1826 | 3175 | Nitrogen | 0.371 | 0.589 | 0.462 |
| 1827 | 3176 | Nitrogen | 0.371 | 0.514 | 0.348 |
| 1832 | 3180 | Nitrogen | 0.370 | 0.511 | 0.363 |
| 1833 | 3181 | Nitrogen | 0.369 | 0.595 | 0.473 |
| 1834 | 3182 | Nitrogen | 0.369 | 0.537 | 0.411 |
| 1836 | 3184 | Nitrogen | 0.366 | 0.512 | 0.491 |
| 1842 | 3190 | Nitrogen | 0.362 | 0.608 | 0.597 |
| 1843 | 3191 | Nitrogen | 0.361 | 0.570 | 0.473 |
| 1844 | 3192 | Nitrogen | 0.361 | 0.548 | 0.507 |
| 1845 | 3193 | Nitrogen | 0.361 | 0.489 | 0.479 |
| 1847 | 3195 | Nitrogen | 0.360 | 0.541 | 0.451 |
| 1848 | 3196 | Nitrogen | 0.359 | 0.548 | 0.507 |
| 1852 | 3200 | Nitrogen | 0.358 | 0.525 | 0.442 |
| 1857 | 3205 | Nitrogen | 0.355 | 0.489 | 0.458 |
| 1858 | 3206 | Nitrogen | 0.355 | 0.489 | 0.456 |
| 1859 | 3207 | Nitrogen | 0.355 | 0.489 | 0.553 |
| 1860 | 3208 | Nitrogen | 0.355 | 0.489 | 0.456 |
| 1861 | 3209 | Nitrogen | 0.355 | 0.489 | 0.456 |
| 1862 | 3210 | Nitrogen | 0.355 | 0.489 | 0.456 |
| 1863 | 3211 | Nitrogen | 0.355 | 0.489 | 0.456 |
| 1866 | 3214 | Nitrogen | 0.354 | 0.529 | 0.456 |
| 1867 | 3215 | Nitrogen | 0.354 | 0.490 | 0.529 |
| 1868 | 3216 | Nitrogen | 0.353 | 0.567 | 0.514 |
| 1871 | 3219 | Nitrogen | 0.353 | 0.608 | 0.479 |
| 1873 | 3221 | Nitrogen | 0.352 | 0.514 | 0.470 |
| 1874 | 3222 | Nitrogen | 0.351 | 0.492 | 0.411 |
| 1880 | 3228 | Nitrogen | 0.348 | 0.568 | 0.417 |
| 1881 | 3229 | Nitrogen | 0.348 | 0.490 | 0.631 |
| 1882 | 3230 | Nitrogen | 0.345 | 0.636 | 0.481 |
| 1884 | 3232 | Nitrogen | 0.345 | 0.501 | 0.547 |
| 1888 | 3235 | Nitrogen | 0.345 | 0.570 | 0.473 |
| 1889 | 3236 | Nitrogen | 0.345 | 0.570 | 0.473 |
| 1890 | 3237 | Nitrogen | 0.345 | 0.570 | 0.473 |
| 1893 | 3240 | Nitrogen | 0.345 | 0.536 | 0.435 |
| 1894 | N.A. | Nitrogen | 0.345 | 0.574 | 0.572 |
| 1895 | N.A. | Nitrogen | 0.345 | 0.574 | 0.572 |
| 1896 | 3241 | Nitrogen | 0.345 | 0.574 | 0.560 |
| 1897 | 3242 | Nitrogen | 0.345 | 0.574 | 0.387 |
| 1898 | 3243 | Nitrogen | 0.345 | 0.574 | 0.387 |
| 1899 | 3244 | Nitrogen | 0.344 | 0.589 | 0.492 |
| 1901 | 3246 | Nitrogen | 0.343 | 0.576 | 0.459 |
| 1903 | 3248 | Nitrogen | 0.340 | 0.495 | 0.438 |
| 1911 | 3256 | Nitrogen | 0.336 | 0.498 | 0.555 |
| 1913 | 3258 | Nitrogen | 0.335 | 0.523 | 0.627 |
| 1914 | 3259 | Nitrogen | 0.334 | 0.841 | 0.271 |
| 1915 | 3260 | Nitrogen | 0.334 | 0.592 | 0.573 |
| 1916 | 3261 | Nitrogen | 0.334 | 0.592 | 0.573 |
| 1923 | 3268 | Nitrogen | 0.332 | 0.540 | 0.318 |
| 1924 | 3269 | Nitrogen | 0.332 | 0.545 | 0.344 |
| 1929 | 3274 | Nitrogen | 0.326 | 0.490 | 0.529 |
| 1930 | 3275 | Nitrogen | 0.326 | 0.490 | 0.507 |
| 1931 | 3276 | Nitrogen | 0.326 | 0.490 | 0.509 |
| 1933 | 3278 | Nitrogen | 0.325 | 0.552 | 0.507 |
| 1939 | 3284 | Nitrogen | 0.322 | 0.501 | 0.547 |
| 1940 | 3285 | Nitrogen | 0.322 | 0.501 | 0.547 |
| 1941 | 3286 | Nitrogen | 0.322 | 0.501 | 0.547 |
| 1945 | 3290 | Nitrogen | 0.320 | 0.493 | 0.379 |
| 1949 | 3293 | Nitrogen | 0.316 | 0.492 | 0.278 |
| 1952 | 3296 | Nitrogen | 0.316 | 0.664 | 0.575 |
| 1954 | 3298 | Nitrogen | 0.315 | 0.548 | 0.451 |
| 1955 | 3299 | Nitrogen | 0.315 | 0.541 | 0.451 |
| 1956 | 3300 | Nitrogen | 0.315 | 0.541 | 0.451 |
| 1958 | 3302 | Nitrogen | 0.312 | 0.574 | 0.402 |
| 1961 | 3304 | Nitrogen | 0.311 | 0.671 | 0.502 |
| 1966 | 3309 | Nitrogen | 0.308 | 0.841 | 0.420 |
| 1969 | N.A. | Nitrogen | 0.306 | 0.528 | 0.328 |
| 1971 | 3312 | Nitrogen | 0.306 | 0.528 | 0.384 |
| 1976 | N.A. | Nitrogen | 0.305 | 0.548 | 0.507 |
| 1977 | 3317 | Nitrogen | 0.305 | 0.548 | 0.507 |
| 1978 | N.A. | Nitrogen | 0.305 | 0.548 | 0.507 |
| 1979 | 3318 | Nitrogen | 0.305 | 0.548 | 0.507 |
| 1980 | 3319 | Nitrogen | 0.305 | 0.548 | 0.507 |
| 1981 | 3320 | Nitrogen | 0.305 | 0.548 | 0.519 |
| 1982 | 3321 | Nitrogen | 0.305 | 0.589 | 0.492 |
| 1983 | 3322 | Nitrogen | 0.305 | 0.589 | 0.492 |
| 1990 | 3329 | Nitrogen | 0.301 | 0.495 | 0.361 |
| 1991 | 3330 | Nitrogen | 0.301 | 0.827 | 0.425 |
| 1999 | 3338 | Nitrogen | 0.300 | 0.493 | 0.619 |
| 2000 | 3339 | Nitrogen | 0.299 | 0.592 | 0.539 |
| 2002 | 3340 | Nitrogen | 0.297 | 0.523 | 0.552 |
| 2004 | 3342 | Nitrogen | 0.296 | 0.535 | 0.318 |
| 2005 | 3343 | Nitrogen | 0.296 | 0.723 | 0.529 |
| 2007 | 3345 | Nitrogen | 0.296 | 0.496 | 0.561 |
| 2009 | 3347 | Nitrogen | 0.296 | 0.749 | 0.338 |
| 2014 | 3352 | Nitrogen | 0.294 | 0.580 | 0.327 |
| 2023 | 3361 | Nitrogen | 0.287 | 0.554 | 0.375 |
| 2025 | 3363 | Nitrogen | 0.287 | 0.701 | 0.399 |
| 2027 | 3365 | Nitrogen | 0.287 | 0.545 | 0.344 |
| 2028 | 3366 | Nitrogen | 0.287 | 0.545 | 0.594 |
| 2029 | 3367 | Nitrogen | 0.287 | 0.545 | 0.456 |
| 2029 | 3367 | Nitrogen | 0.287 | 0.545 | 0.456 |
| 2030 | 3368 | Nitrogen | 0.287 | 0.545 | 0.456 |
| 2031 | N.A. | Nitrogen | 0.287 | 0.545 | 0.344 |
| 2032 | 3369 | Nitrogen | 0.287 | 0.545 | 0.344 |
| 2033 | 3370 | Nitrogen | 0.287 | 0.669 | 0.533 |
| 2035 | 3372 | Nitrogen | 0.287 | 0.678 | 0.427 |
| 2038 | 3375 | Nitrogen | 0.286 | 0.493 | 0.318 |
| 2041 | 3378 | Nitrogen | 0.285 | 0.733 | 0.376 |
| 2042 | 3379 | Nitrogen | 0.285 | 0.733 | 0.376 |
| 2053 | 3389 | Nitrogen | 0.284 | 0.495 | 0.450 |
| 2066 | 3401 | Nitrogen | 0.278 | 0.593 | 0.414 |
| 2067 | 3402 | Nitrogen | 0.278 | 0.559 | 0.289 |
| 2068 | 3403 | Nitrogen | 0.276 | 0.498 | 0.547 |
| 2081 | 3416 | Nitrogen | 0.273 | 0.523 | 0.552 |
| 2082 | 3417 | Nitrogen | 0.273 | 0.523 | 0.466 |
| 2083 | 3418 | Nitrogen | 0.273 | 0.523 | 0.466 |
| 2084 | 3419 | Nitrogen | 0.273 | 0.518 | 0.466 |
| 2088 | 3423 | Nitrogen | 0.270 | 0.580 | 0.321 |
| 2091 | 3426 | Nitrogen | 0.270 | 0.586 | 0.591 |
| 2092 | 3427 | Nitrogen | 0.270 | 0.490 | 0.627 |
| 2093 | 3428 | Nitrogen | 0.269 | 0.532 | 0.000 |
| 2094 | 3429 | Nitrogen | 0.268 | 0.541 | 0.405 |
| 2099 | 3432 | Nitrogen | 0.260 | 0.747 | 0.494 |
| 2100 | 3433 | Nitrogen | 0.260 | 0.685 | 0.557 |
| 2101 | 3434 | Nitrogen | 0.259 | 0.490 | 0.498 |
| 2108 | 3441 | Nitrogen | 0.256 | 0.841 | 0.337 |
| 2111 | 3442 | Nitrogen | 0.256 | 0.582 | 0.335 |
| 2112 | 3443 | Nitrogen | 0.253 | 0.621 | 0.408 |
| 2117 | 3448 | Nitrogen | 0.252 | 0.701 | 0.478 |
| 2118 | 3449 | Nitrogen | 0.251 | 0.546 | 0.428 |
| 2119 | 3450 | Nitrogen | 0.250 | 0.502 | 0.561 |
| 2120 | 3451 | Nitrogen | 0.249 | 0.537 | 0.378 |
| 2126 | 3457 | Nitrogen | 0.246 | 0.563 | 0.289 |
| 2127 | 3458 | Nitrogen | 0.245 | 0.490 | 0.627 |
| 2134 | 3465 | Nitrogen | 0.242 | 0.542 | 0.571 |
| 2135 | 3466 | Nitrogen | 0.239 | 0.515 | 0.318 |
| 2136 | 3467 | Nitrogen | 0.238 | 0.644 | 0.506 |
| 2142 | 3473 | Nitrogen | 0.235 | 0.583 | 0.390 |
| 2151 | 3481 | Nitrogen | 0.220 | 0.513 | 0.318 |
| 2157 | 3487 | Nitrogen | 0.219 | 0.753 | 0.431 |
| 2162 | 3492 | Nitrogen | 0.213 | 0.781 | 0.478 |
| 2163 | 3493 | Nitrogen | 0.211 | 0.493 | 0.411 |
| 2166 | 3496 | Nitrogen | 0.210 | 0.490 | 0.487 |
| 2170 | 3499 | Nitrogen | 0.205 | 0.522 | 0.478 |
| 2176 | 3505 | Nitrogen | 0.204 | 0.559 | 0.558 |
| 2177 | 3506 | Nitrogen | 0.204 | 0.559 | 0.558 |
| 2178 | 3507 | Nitrogen | 0.204 | 0.559 | 0.289 |
| 2179 | 3508 | Nitrogen | 0.204 | 0.559 | 0.289 |
| 2193 | 3522 | Nitrogen | 0.184 | 0.659 | 0.376 |
| 2194 | 3523 | Nitrogen | 0.180 | 0.520 | 0.443 |
| 2195 | 3524 | Nitrogen | 0.180 | 0.532 | 0.000 |
| 2196 | 3525 | Nitrogen | 0.179 | 0.490 | 0.627 |
| 2197 | 3526 | Nitrogen | 0.179 | 0.490 | 0.498 |
| 2198 | 3527 | Nitrogen | 0.179 | 0.490 | 0.487 |
| 2205 | 3534 | Nitrogen | 0.173 | 0.604 | 0.362 |
| 2209 | 3537 | Nitrogen | 0.164 | 0.830 | 0.523 |
| 2210 | 3538 | Nitrogen | 0.163 | 0.605 | 0.477 |
| 2211 | 3539 | Nitrogen | 0.160 | 0.702 | 0.420 |
| 2213 | 3541 | Nitrogen | 0.160 | 0.589 | 0.630 |
| 2217 | 3545 | Nitrogen | 0.153 | 0.534 | 0.305 |
| 2223 | 3551 | Nitrogen | 0.146 | 0.565 | 0.465 |
| 2224 | 3552 | Nitrogen | 0.146 | 0.532 | 0.000 |
| 2230 | 3558 | Nitrogen | 0.144 | 0.589 | 0.574 |
| 2236 | 3564 | Nitrogen | 0.132 | 0.674 | 0.526 |
| 2238 | 3566 | Nitrogen | 0.129 | 0.617 | 0.560 |
| 2241 | 3569 | Nitrogen | 0.125 | 0.518 | 0.605 |
| 2243 | 3571 | Nitrogen | 0.125 | 0.498 | 0.479 |
| 2247 | 3575 | Nitrogen | 0.114 | 0.510 | 0.442 |
| 2248 | 3576 | Nitrogen | 0.111 | 0.504 | 0.308 |
| 2249 | 3577 | Nitrogen | 0.111 | 0.509 | 0.478 |
| 2250 | 3578 | Nitrogen | 0.111 | 0.509 | 0.478 |
| 2251 | 3579 | Nitrogen | 0.108 | 0.530 | 0.374 |
| 2253 | 3581 | Nitrogen | 0.105 | 0.793 | 0.147 |
| 2254 | 3582 | Nitrogen | 0.105 | 0.582 | 0.507 |
| 2262 | 3590 | Nitrogen | 0.094 | 0.771 | 0.174 |
| 2270 | 3598 | Nitrogen | 0.086 | 0.549 | 0.395 |
| 2273 | 3601 | Nitrogen | 0.047 | 0.507 | 0.404 |
| 2275 | 3603 | Nitrogen | 0.047 | 0.546 | 0.536 |
| 2276 | 3604 | Nitrogen | 0.047 | 0.568 | 0.536 |
| 2279 | 3607 | Nitrogen | 0.047 | 0.805 | 0.474 |
| 2283 | 3610 | Nitrogen | 0.047 | 0.509 | 0.630 |
| 2284 | 3611 | Nitrogen | 0.047 | 0.509 | 0.478 |
| 2286 | 3613 | Nitrogen | 0.047 | 0.643 | 0.243 |
| 2287 | 3614 | Nitrogen | 0.045 | 0.592 | 0.496 |
| 2289 | 3616 | Nitrogen | 0.045 | 0.510 | 0.442 |
| 2290 | 3617 | Nitrogen | 0.045 | 0.510 | 0.442 |
| 2292 | 3619 | Nitrogen | 0.045 | 0.510 | 0.442 |
| 2293 | N.A. | Nitrogen | 0.045 | 0.529 | 0.456 |
| 2294 | 3620 | Nitrogen | 0.045 | 0.529 | 0.456 |
| 2295 | 3621 | Nitrogen | 0.045 | 0.529 | 0.456 |
| 2296 | 3622 | Nitrogen | 0.043 | 0.504 | 0.386 |
| 2300 | 3626 | Nitrogen | 0.043 | 0.668 | 0.386 |
| 2301 | 3627 | Nitrogen | 0.043 | 0.496 | 0.612 |
| 2303 | 3629 | Nitrogen | 0.043 | 0.589 | 0.295 |
| 2305 | 3631 | Nitrogen | 0.021 | 0.911 | 0.436 |
| 2307 | 3633 | Nitrogen | 0.021 | 0.589 | 0.345 |
| 2308 | 3634 | Nitrogen | 0.013 | 0.530 | 0.374 |
| 2309 | 3635 | Nitrogen | 0.013 | 0.496 | 0.428 |
| 2314 | 3640 | Nitrogen | 0.000 | 0.673 | 0.147 |
| 2315 | 3641 | Nitrogen | 0.000 | 0.712 | 0.636 |
| 2316 | 3642 | Nitrogen | 0.000 | 0.692 | 0.560 |
| 2320 | 3646 | Nitrogen | 0.000 | 0.496 | 0.443 |
| 2321 | 3647 | Nitrogen | 0.000 | 0.496 | 0.443 |
| 2322 | 3648 | Nitrogen | 0.000 | 0.496 | 0.514 |
| 2324 | 3650 | Nitrogen | 0.000 | 0.814 | 0.335 |
| 2325 | 3651 | Nitrogen | 0.000 | 0.589 | 0.434 |
| 2327 | 3653 | Nitrogen | 0.000 | 0.579 | 0.564 |
| 2328 | 3654 | Nitrogen | 0.000 | 0.634 | 0.377 |
| 2329 | 3655 | Nitrogen | 0.000 | 0.858 | 0.000 |
| 2330 | 3656 | Nitrogen | 0.000 | 0.549 | 0.000 |
| 2331 | 3657 | Nitrogen | 0.000 | 0.825 | 0.000 |
| 2343 | 3669 | Nitrogen | 0.000 | 0.530 | 0.374 |
| 2344 | 3670 | Nitrogen | 0.000 | 0.530 | 0.374 |
| 2345 | 3671 | Nitrogen | 0.000 | 0.530 | 0.374 |
| 2346 | 3672 | Nitrogen | 0.000 | 0.530 | 0.636 |
| 2347 | 3673 | Nitrogen | 0.000 | 0.530 | 0.374 |
| 2348 | 3674 | Nitrogen | 0.000 | 0.851 | 0.528 |
| 2352 | 3677 | Nitrogen | 0.000 | 0.692 | 0.560 |
| 2353 | 3678 | Nitrogen | 0.000 | 0.692 | 0.560 |
| 2355 | 3680 | Nitrogen | 0.000 | 0.770 | 0.481 |
| 2358 | 3683 | Nitrogen | 0.000 | 0.779 | 0.478 |
| 2360 | 3685 | Nitrogen | 0.000 | 0.606 | 0.147 |
| 2365 | 3690 | Nitrogen | 0.000 | 0.565 | 0.465 |
| 2366 | 3691 | Nitrogen | 0.000 | 0.565 | 0.465 |
| 2367 | 3692 | Nitrogen | 0.000 | 0.565 | 0.465 |
| 2368 | 3693 | Nitrogen | 0.000 | 0.571 | 0.578 |
| 2369 | 3694 | Nitrogen | 0.000 | 0.550 | 0.520 |
| 2370 | 3695 | Nitrogen | 0.000 | 0.550 | 0.520 |
| 2384 | 3706 | Nitrogen | 0.000 | 0.563 | 0.215 |
| 2385 | 3707 | Nitrogen | 0.000 | 0.713 | 0.554 |
| 2393 | 3715 | Nitrogen | 0.000 | 0.597 | 0.328 |
| 2394 | 3716 | Nitrogen | 0.000 | 0.597 | 0.328 |
| 2395 | 3717 | Nitrogen | 0.000 | 0.597 | 0.328 |
| 2415 | N.A. | Nitrogen | 0.000 | 0.668 | 0.383 |
| 2416 | N.A. | Nitrogen | 0.000 | 0.668 | 0.383 |
| 2418 | 3735 | Nitrogen | 0.000 | 0.542 | 0.517 |
| 2419 | 3736 | Nitrogen | 0.000 | 0.701 | 0.595 |
| 2420 | 3737 | Nitrogen | 0.000 | 0.582 | 0.507 |
| 2421 | 3738 | Nitrogen | 0.000 | 0.582 | 0.507 |
| 2422 | 3739 | Nitrogen | 0.000 | 0.496 | 0.562 |
| 2423 | N.A. | Nitrogen | 0.000 | 0.496 | 0.605 |
| 2424 | N.A. | Nitrogen | 0.000 | 0.496 | 0.605 |
| 2425 | N.A. | Nitrogen | 0.000 | 0.747 | 0.542 |
| 2427 | 3741 | Nitrogen | 0.000 | 0.634 | 0.398 |
| 2432 | 3745 | Nitrogen | 0.000 | 0.528 | 0.611 |
| 2433 | 3746 | Nitrogen | 0.000 | 0.583 | 0.437 |
| 2442 | 3755 | Nitrogen | 0.000 | 0.662 | 0.336 |
| 2442 | 3755 | Nitrogen | 0.000 | 0.662 | 0.336 |
| 2444 | 3757 | Nitrogen | 0.000 | 0.661 | 0.572 |
| 2446 | 3759 | Nitrogen | 0.000 | 0.858 | 0.304 |
| 2454 | 3766 | Nitrogen | 0.000 | 0.710 | 0.567 |
| 2455 | 3767 | Nitrogen | 0.000 | 0.522 | 0.478 |
| 2456 | 3768 | Nitrogen | 0.000 | 0.522 | 0.478 |
| 2457 | 3769 | Nitrogen | 0.000 | 0.522 | 0.478 |
| 2470 | 3782 | Nitrogen | 0.000 | 0.644 | 0.506 |
| 2471 | 3783 | Nitrogen | 0.000 | 0.644 | 0.506 |
| 2472 | 3784 | Nitrogen | 0.000 | 0.532 | 0.000 |
| 2473 | 3785 | Nitrogen | 0.000 | 0.532 | 0.000 |
| 2474 | 3786 | Nitrogen | 0.000 | 0.532 | 0.000 |
| 2475 | 3787 | Nitrogen | 0.000 | 0.532 | 0.000 |
| 2476 | 3788 | Nitrogen | 0.000 | 0.532 | 0.000 |
| 2492 | 3802 | Nitrogen | 0.000 | 0.589 | 0.574 |
| 2493 | 3803 | Nitrogen | 0.000 | 0.589 | 0.539 |
| 2494 | N.A. | Nitrogen | 0.000 | 0.589 | 0.539 |
| 2495 | 3804 | Nitrogen | 0.000 | 0.589 | 0.491 |
| 1127 | 2496 | Drought | 0.763 | 0.325 | 0.354 |
| 1131 | 2500 | Drought | 0.745 | 0.264 | 0.496 |
| 1133 | 2502 | Drought | 0.593 | 0.256 | 0.410 |
| 1139 | 2508 | Drought | 0.553 | 0.233 | 0.564 |
| 1140 | 2509 | Drought | 0.706 | 0.384 | 0.472 |
| 1144 | 2513 | Drought | 0.463 | 0.301 | 0.466 |
| 1148 | 2517 | Drought | 0.475 | 0.000 | 0.559 |
| 1150 | 2519 | Drought | 0.714 | 0.212 | 0.594 |
| 1151 | 2520 | Drought | 0.510 | 0.278 | 0.499 |
| 1158 | 2527 | Drought | 0.476 | 0.270 | 0.603 |
| 1175 | 2544 | Drought | 0.851 | 0.173 | 0.394 |
| 1176 | 2545 | Drought | 0.514 | 0.000 | 0.527 |
| 1184 | 2553 | Drought | 0.465 | 0.210 | 0.309 |
| 1188 | 2557 | Drought | 0.839 | 0.324 | 0.579 |
| 1201 | 2570 | Drought | 0.593 | 0.272 | 0.442 |
| 1202 | 2571 | Drought | 0.465 | 0.357 | 0.185 |
| 1205 | 2574 | Drought | 0.474 | 0.279 | 0.629 |
| 1209 | 2578 | Drought | 0.777 | 0.000 | 0.538 |
| 1213 | 2582 | Drought | 0.516 | 0.314 | 0.429 |
| 1221 | 2590 | Drought | 0.467 | 0.243 | 0.501 |
| 1222 | 2591 | Drought | 0.482 | 0.456 | 0.584 |
| 1223 | 2592 | Drought | 0.527 | 0.258 | 0.409 |
| 1224 | 2593 | Drought | 0.482 | 0.222 | 0.446 |
| 1227 | 2596 | Drought | 0.488 | 0.247 | 0.540 |
| 1228 | 2597 | Drought | 0.481 | 0.212 | 0.555 |
| 1232 | 2601 | Drought | 0.656 | 0.453 | 0.542 |
| 1236 | 2605 | Drought | 0.554 | 0.338 | 0.457 |
| 1238 | 2607 | Drought | 0.673 | 0.316 | 0.430 |
| 1239 | 2608 | Drought | 0.570 | 0.212 | 0.126 |
| 1240 | 2609 | Drought | 0.626 | 0.340 | 0.459 |
| 1243 | 2612 | Drought | 0.541 | 0.000 | 0.355 |
| 1247 | 2616 | Drought | 0.576 | 0.180 | 0.578 |
| 1251 | 2620 | Drought | 0.924 | 0.359 | 0.461 |
| 1252 | 2621 | Drought | 0.921 | 0.334 | 0.526 |
| 1253 | 2622 | Drought | 0.919 | 0.000 | 0.410 |
| 1257 | 2626 | Drought | 0.860 | 0.000 | 0.390 |
| 1259 | 2628 | Drought | 0.844 | 0.429 | 0.419 |
| 1261 | 2630 | Drought | 0.779 | 0.000 | 0.410 |
| 1262 | 2631 | Drought | 0.756 | 0.393 | 0.385 |
| 1264 | 2633 | Drought | 0.733 | 0.274 | 0.392 |
| 1266 | 2635 | Drought | 0.712 | 0.000 | 0.448 |
| 1268 | 2637 | Drought | 0.707 | 0.098 | 0.432 |
| 1269 | 2638 | Drought | 0.707 | 0.098 | 0.432 |
| 1270 | 2639 | Drought | 0.703 | 0.301 | 0.317 |
| 1272 | 2641 | Drought | 0.701 | 0.280 | 0.440 |
| 1273 | 2642 | Drought | 0.700 | 0.280 | 0.440 |
| 1274 | 2643 | Drought | 0.694 | 0.467 | 0.628 |
| 1276 | N.A. | Drought | 0.693 | 0.000 | 0.210 |
| 1277 | 2645 | Drought | 0.692 | 0.116 | 0.318 |
| 1280 | 2648 | Drought | 0.656 | 0.171 | 0.560 |
| 1281 | 2649 | Drought | 0.653 | 0.167 | 0.522 |
| 1282 | 2650 | Drought | 0.653 | 0.221 | 0.388 |
| 1285 | 2653 | Drought | 0.650 | 0.378 | 0.604 |
| 1286 | 2654 | Drought | 0.648 | 0.122 | 0.529 |
| 1287 | 2655 | Drought | 0.647 | 0.221 | 0.388 |
| 1288 | 2656 | Drought | 0.646 | 0.279 | 0.634 |
| 1289 | 2657 | Drought | 0.644 | 0.229 | 0.351 |
| 1291 | 2659 | Drought | 0.642 | 0.000 | 0.404 |
| 1293 | 2661 | Drought | 0.638 | 0.309 | 0.586 |
| 1294 | 2662 | Drought | 0.637 | 0.466 | 0.387 |
| 1295 | 2663 | Drought | 0.633 | 0.307 | 0.574 |
| 1297 | 2665 | Drought | 0.631 | 0.438 | 0.333 |
| 1298 | 2666 | Drought | 0.630 | 0.000 | 0.564 |
| 1300 | 2668 | Drought | 0.624 | 0.000 | 0.446 |
| 1301 | 2669 | Drought | 0.623 | 0.000 | 0.446 |
| 1302 | 2670 | Drought | 0.623 | 0.000 | 0.440 |
| 1303 | 2671 | Drought | 0.623 | 0.000 | 0.440 |
| 1304 | 2672 | Drought | 0.623 | 0.000 | 0.440 |
| 1305 | 2673 | Drought | 0.623 | 0.000 | 0.440 |
| 1306 | 2674 | Drought | 0.621 | 0.378 | 0.604 |
| 1309 | 2677 | Drought | 0.614 | 0.000 | 0.384 |
| 1310 | 2678 | Drought | 0.612 | 0.309 | 0.586 |
| 1311 | 2679 | Drought | 0.612 | 0.309 | 0.586 |
| 1312 | 2680 | Drought | 0.609 | 0.000 | 0.588 |
| 1313 | 2681 | Drought | 0.607 | 0.339 | 0.372 |
| 1314 | 2682 | Drought | 0.604 | 0.212 | 0.000 |
| 1315 | 2683 | Drought | 0.604 | 0.000 | 0.544 |
| 1316 | 2684 | Drought | 0.602 | 0.167 | 0.353 |
| 1318 | 2686 | Drought | 0.601 | 0.239 | 0.370 |
| 1319 | 2687 | Drought | 0.601 | 0.410 | 0.543 |
| 1320 | N.A. | Drought | 0.599 | 0.278 | 0.605 |
| 1321 | N.A. | Drought | 0.599 | 0.278 | 0.605 |
| 1323 | 2689 | Drought | 0.597 | 0.287 | 0.437 |
| 1324 | 2690 | Drought | 0.597 | 0.287 | 0.437 |
| 1325 | 2691 | Drought | 0.592 | 0.475 | 0.319 |
| 1326 | 2692 | Drought | 0.592 | 0.338 | 0.301 |
| 1327 | 2693 | Drought | 0.590 | 0.256 | 0.415 |
| 1328 | 2694 | Drought | 0.590 | 0.255 | 0.482 |
| 1330 | 2696 | Drought | 0.586 | 0.000 | 0.401 |
| 1331 | 2697 | Drought | 0.585 | 0.000 | 0.404 |
| 1332 | 2698 | Drought | 0.583 | 0.000 | 0.404 |
| 1333 | 2699 | Drought | 0.583 | 0.000 | 0.404 |
| 1334 | 2700 | Drought | 0.583 | 0.000 | 0.590 |
| 1335 | 2701 | Drought | 0.581 | 0.000 | 0.444 |
| 1337 | 2703 | Drought | 0.580 | 0.229 | 0.383 |
| 1338 | 2704 | Drought | 0.580 | 0.444 | 0.343 |
| 1340 | 2706 | Drought | 0.579 | 0.000 | 0.405 |
| 1341 | N.A. | Drought | 0.579 | 0.299 | 0.386 |
| 1342 | N.A. | Drought | 0.579 | 0.299 | 0.386 |
| 1342 | N.A. | Drought | 0.461 | 0.168 | 0.542 |
| 1343 | 2707 | Drought | 0.579 | 0.299 | 0.386 |
| 1344 | 2708 | Drought | 0.578 | 0.000 | 0.408 |
| 1345 | 2709 | Drought | 0.575 | 0.000 | 0.618 |
| 1346 | 2710 | Drought | 0.575 | 0.000 | 0.618 |
| 1348 | 2712 | Drought | 0.574 | 0.247 | 0.295 |
| 1349 | 2713 | Drought | 0.574 | 0.331 | 0.346 |
| 1350 | 2714 | Drought | 0.574 | 0.198 | 0.421 |
| 1351 | 2715 | Drought | 0.573 | 0.228 | 0.432 |
| 1352 | 2716 | Drought | 0.572 | 0.444 | 0.630 |
| 1354 | 2718 | Drought | 0.572 | 0.000 | 0.000 |
| 1357 | 2721 | Drought | 0.568 | 0.171 | 0.440 |
| 1358 | 2722 | Drought | 0.568 | 0.352 | 0.367 |
| 1359 | 2723 | Drought | 0.565 | 0.228 | 0.000 |
| 1360 | 2724 | Drought | 0.565 | 0.455 | 0.576 |
| 1364 | 2728 | Drought | 0.563 | 0.455 | 0.582 |
| 1365 | 2729 | Drought | 0.561 | 0.419 | 0.383 |
| 1366 | 2730 | Drought | 0.560 | 0.409 | 0.471 |
| 1367 | 2731 | Drought | 0.557 | 0.281 | 0.371 |
| 1368 | 2732 | Drought | 0.557 | 0.228 | 0.432 |
| 1369 | 2733 | Drought | 0.556 | 0.460 | 0.466 |
| 1369 | 2733 | Drought | 0.556 | 0.460 | 0.466 |
| 1369 | 2733 | Drought | 0.550 | 0.460 | 0.307 |
| 1370 | 2734 | Drought | 0.556 | 0.361 | 0.369 |
| 1371 | 2735 | Drought | 0.556 | 0.000 | 0.614 |
| 1372 | 2736 | Drought | 0.555 | 0.000 | 0.614 |
| 1374 | 2737 | Drought | 0.555 | 0.000 | 0.614 |
| 1375 | 2738 | Drought | 0.555 | 0.000 | 0.614 |
| 1376 | 2739 | Drought | 0.554 | 0.347 | 0.516 |
| 1377 | 2740 | Drought | 0.551 | 0.247 | 0.387 |
| 1380 | 2743 | Drought | 0.549 | 0.475 | 0.575 |
| 1381 | 2744 | Drought | 0.549 | 0.000 | 0.400 |
| 1382 | 2745 | Drought | 0.548 | 0.278 | 0.479 |
| 1383 | 2746 | Drought | 0.548 | 0.347 | 0.516 |
| 1384 | 2747 | Drought | 0.547 | 0.173 | 0.339 |
| 1385 | 2748 | Drought | 0.546 | 0.355 | 0.423 |
| 1386 | 2749 | Drought | 0.546 | 0.000 | 0.417 |
| 1387 | 2750 | Drought | 0.545 | 0.255 | 0.609 |
| 1388 | 2751 | Drought | 0.545 | 0.301 | 0.493 |
| 1391 | 2754 | Drought | 0.544 | 0.382 | 0.462 |
| 1392 | 2755 | Drought | 0.544 | 0.274 | 0.344 |
| 1393 | 2756 | Drought | 0.543 | 0.122 | 0.321 |
| 1395 | 2758 | Drought | 0.541 | 0.000 | 0.399 |
| 1397 | 2760 | Drought | 0.540 | 0.336 | 0.471 |
| 1398 | 2761 | Drought | 0.539 | 0.293 | 0.610 |
| 1399 | 2762 | Drought | 0.539 | 0.256 | 0.504 |
| 1400 | 2763 | Drought | 0.538 | 0.301 | 0.440 |
| 1401 | N.A. | Drought | 0.538 | 0.301 | 0.462 |
| 1402 | 2764 | Drought | 0.537 | 0.212 | 0.588 |
| 1403 | 2765 | Drought | 0.537 | 0.228 | 0.422 |
| 1404 | 2766 | Drought | 0.536 | 0.000 | 0.000 |
| 1405 | 2767 | Drought | 0.536 | 0.233 | 0.594 |
| 1406 | 2768 | Drought | 0.536 | 0.199 | 0.395 |
| 1407 | 2769 | Drought | 0.535 | 0.000 | 0.474 |
| 1410 | 2772 | Drought | 0.534 | 0.168 | 0.499 |
| 1411 | 2773 | Drought | 0.533 | 0.418 | 0.393 |
| 1412 | 2773 | Drought | 0.533 | 0.418 | 0.393 |
| 1413 | 2774 | Drought | 0.533 | 0.229 | 0.351 |
| 1414 | 2775 | Drought | 0.533 | 0.484 | 0.559 |
| 1416 | 2777 | Drought | 0.532 | 0.315 | 0.554 |
| 1417 | 2778 | Drought | 0.532 | 0.000 | 0.422 |
| 1418 | 2779 | Drought | 0.532 | 0.000 | 0.422 |
| 1420 | 2781 | Drought | 0.532 | 0.408 | 0.432 |
| 1421 | 2782 | Drought | 0.532 | 0.000 | 0.422 |
| 1422 | 2783 | Drought | 0.532 | 0.000 | 0.422 |
| 1423 | 2784 | Drought | 0.531 | 0.199 | 0.602 |
| 1424 | 2785 | Drought | 0.531 | 0.000 | 0.514 |
| 1425 | 2786 | Drought | 0.531 | 0.239 | 0.338 |
| 1426 | 2787 | Drought | 0.530 | 0.000 | 0.497 |
| 1427 | 2788 | Drought | 0.529 | 0.200 | 0.000 |
| 1428 | 2789 | Drought | 0.529 | 0.454 | 0.326 |
| 1433 | 2794 | Drought | 0.528 | 0.321 | 0.454 |
| 1439 | 2800 | Drought | 0.528 | 0.336 | 0.301 |
| 1440 | 2801 | Drought | 0.527 | 0.000 | 0.508 |
| 1442 | 2803 | Drought | 0.527 | 0.098 | 0.590 |
| 1443 | 2804 | Drought | 0.527 | 0.462 | 0.559 |
| 1444 | 2805 | Drought | 0.527 | 0.462 | 0.561 |
| 1445 | 2806 | Drought | 0.527 | 0.462 | 0.561 |
| 1446 | 2807 | Drought | 0.527 | 0.462 | 0.561 |
| 1447 | 2808 | Drought | 0.526 | 0.255 | 0.525 |
| 1450 | 2810 | Drought | 0.525 | 0.264 | 0.473 |
| 1455 | 2815 | Drought | 0.523 | 0.000 | 0.497 |
| 1456 | 2816 | Drought | 0.523 | 0.000 | 0.514 |
| 1457 | 2817 | Drought | 0.523 | 0.000 | 0.514 |
| 1461 | 2821 | Drought | 0.523 | 0.358 | 0.603 |
| 1463 | 2823 | Drought | 0.523 | 0.000 | 0.000 |
| 1464 | 2824 | Drought | 0.523 | 0.287 | 0.396 |
| 1465 | 2825 | Drought | 0.522 | 0.198 | 0.476 |
| 1466 | 2826 | Drought | 0.522 | 0.278 | 0.526 |
| 1467 | 2827 | Drought | 0.522 | 0.000 | 0.100 |
| 1469 | 2829 | Drought | 0.522 | 0.301 | 0.555 |
| 1470 | 2830 | Drought | 0.522 | 0.000 | 0.372 |
| 1482 | 2838 | Drought | 0.521 | 0.000 | 0.511 |
| 1483 | 2839 | Drought | 0.520 | 0.228 | 0.477 |
| 1484 | 2840 | Drought | 0.520 | 0.301 | 0.578 |
| 1485 | 2841 | Drought | 0.520 | 0.301 | 0.578 |
| 1488 | 2844 | Drought | 0.520 | 0.301 | 0.551 |
| 1489 | 2845 | Drought | 0.519 | 0.419 | 0.416 |
| 1490 | 2846 | Drought | 0.518 | 0.168 | 0.559 |
| 1492 | 2848 | Drought | 0.517 | 0.198 | 0.524 |
| 1494 | 2850 | Drought | 0.517 | 0.000 | 0.556 |
| 1495 | 2851 | Drought | 0.517 | 0.228 | 0.537 |
| 1496 | 2852 | Drought | 0.516 | 0.000 | 0.451 |
| 1497 | 2853 | Drought | 0.514 | 0.000 | 0.462 |
| 1499 | 2855 | Drought | 0.514 | 0.416 | 0.298 |
| 1500 | 2856 | Drought | 0.513 | 0.270 | 0.478 |
| 1502 | 2858 | Drought | 0.513 | 0.448 | 0.396 |
| 1503 | 2859 | Drought | 0.512 | 0.000 | 0.000 |
| 1504 | 2860 | Drought | 0.512 | 0.294 | 0.556 |
| 1506 | 2862 | Drought | 0.511 | 0.314 | 0.452 |
| 1507 | 2863 | Drought | 0.511 | 0.255 | 0.450 |
| 1508 | N.A. | Drought | 0.511 | 0.255 | 0.450 |
| 1509 | 2864 | Drought | 0.511 | 0.280 | 0.590 |
| 1510 | 2865 | Drought | 0.511 | 0.376 | 0.550 |
| 1511 | 2866 | Drought | 0.511 | 0.331 | 0.378 |
| 1512 | 2867 | Drought | 0.509 | 0.294 | 0.422 |
| 1513 | 2868 | Drought | 0.508 | 0.000 | 0.556 |
| 1514 | 2869 | Drought | 0.508 | 0.278 | 0.400 |
| 1515 | 2870 | Drought | 0.508 | 0.000 | 0.409 |
| 1516 | 2871 | Drought | 0.507 | 0.339 | 0.405 |
| 1517 | 2872 | Drought | 0.507 | 0.378 | 0.573 |
| 1518 | 2873 | Drought | 0.507 | 0.319 | 0.415 |
| 1519 | 2874 | Drought | 0.507 | 0.168 | 0.531 |
| 1520 | 2875 | Drought | 0.507 | 0.256 | 0.450 |
| 1521 | 2876 | Drought | 0.507 | 0.000 | 0.000 |
| 1522 | 2877 | Drought | 0.507 | 0.000 | 0.524 |
| 1523 | 2878 | Drought | 0.507 | 0.256 | 0.382 |
| 1524 | 2879 | Drought | 0.506 | 0.000 | 0.364 |
| 1525 | 2880 | Drought | 0.506 | 0.000 | 0.556 |
| 1526 | 2881 | Drought | 0.506 | 0.000 | 0.556 |
| 1527 | 2882 | Drought | 0.506 | 0.000 | 0.000 |
| 1528 | 2883 | Drought | 0.506 | 0.225 | 0.587 |
| 1530 | 2885 | Drought | 0.505 | 0.305 | 0.374 |
| 1531 | 2886 | Drought | 0.505 | 0.167 | 0.509 |
| 1532 | 2887 | Drought | 0.504 | 0.000 | 0.524 |
| 1534 | 2889 | Drought | 0.503 | 0.255 | 0.411 |
| 1535 | 2890 | Drought | 0.503 | 0.000 | 0.497 |
| 1536 | 2891 | Drought | 0.503 | 0.294 | 0.000 |
| 1538 | 2893 | Drought | 0.502 | 0.279 | 0.374 |
| 1539 | 2894 | Drought | 0.502 | 0.167 | 0.353 |
| 1541 | 2896 | Drought | 0.501 | 0.458 | 0.396 |
| 1542 | 2897 | Drought | 0.501 | 0.473 | 0.396 |
| 1543 | 2898 | Drought | 0.501 | 0.168 | 0.531 |
| 1544 | 2899 | Drought | 0.501 | 0.168 | 0.624 |
| 1545 | 2900 | Drought | 0.501 | 0.168 | 0.624 |
| 1546 | 2901 | Drought | 0.501 | 0.305 | 0.374 |
| 1547 | 2902 | Drought | 0.500 | 0.448 | 0.396 |
| 1548 | 2903 | Drought | 0.500 | 0.448 | 0.569 |
| 1549 | 2904 | Drought | 0.500 | 0.000 | 0.491 |
| 1550 | 2905 | Drought | 0.500 | 0.448 | 0.396 |
| 1551 | 2906 | Drought | 0.500 | 0.000 | 0.364 |
| 1553 | 2908 | Drought | 0.499 | 0.000 | 0.448 |
| 1554 | 2909 | Drought | 0.499 | 0.212 | 0.579 |
| 1555 | 2910 | Drought | 0.498 | 0.000 | 0.000 |
| 1556 | 2911 | Drought | 0.498 | 0.171 | 0.040 |
| 1557 | 2912 | Drought | 0.498 | 0.326 | 0.313 |
| 1558 | 2913 | Drought | 0.498 | 0.307 | 0.401 |
| 1559 | 2914 | Drought | 0.498 | 0.167 | 0.353 |
| 1560 | 2915 | Drought | 0.498 | 0.000 | 0.364 |
| 1561 | 2916 | Drought | 0.498 | 0.000 | 0.000 |
| 1564 | 2919 | Drought | 0.497 | 0.448 | 0.569 |
| 1565 | 2920 | Drought | 0.497 | 0.448 | 0.396 |
| 1566 | 2921 | Drought | 0.497 | 0.448 | 0.396 |
| 1567 | 2922 | Drought | 0.497 | 0.448 | 0.396 |
| 1568 | 2923 | Drought | 0.497 | 0.448 | 0.396 |
| 1569 | 2924 | Drought | 0.496 | 0.386 | 0.493 |
| 1570 | 2925 | Drought | 0.495 | 0.256 | 0.447 |
| 1571 | 2926 | Drought | 0.495 | 0.000 | 0.000 |
| 1572 | 2927 | Drought | 0.495 | 0.168 | 0.499 |
| 1573 | 2928 | Drought | 0.493 | 0.000 | 0.506 |
| 1574 | 2929 | Drought | 0.492 | 0.488 | 0.452 |
| 1575 | 2930 | Drought | 0.470 | 0.000 | 0.000 |
| 1576 | 2931 | Drought | 0.491 | 0.386 | 0.319 |
| 1577 | 2932 | Drought | 0.491 | 0.247 | 0.551 |
| 1578 | 2933 | Drought | 0.491 | 0.173 | 0.333 |
| 1579 | 2934 | Drought | 0.490 | 0.333 | 0.453 |
| 1580 | 2935 | Drought | 0.490 | 0.167 | 0.419 |
| 1582 | 2937 | Drought | 0.488 | 0.000 | 0.000 |
| 1583 | 2938 | Drought | 0.488 | 0.000 | 0.314 |
| 1585 | 2940 | Drought | 0.486 | 0.000 | 0.486 |
| 1586 | 2941 | Drought | 0.486 | 0.296 | 0.323 |
| 1589 | 2944 | Drought | 0.485 | 0.345 | 0.382 |
| 1590 | 2945 | Drought | 0.484 | 0.228 | 0.000 |
| 1591 | 2946 | Drought | 0.484 | 0.000 | 0.382 |
| 1594 | 2949 | Drought | 0.482 | 0.000 | 0.382 |
| 1595 | 2950 | Drought | 0.482 | 0.000 | 0.382 |
| 1596 | 2951 | Drought | 0.482 | 0.000 | 0.382 |
| 1597 | 2952 | Drought | 0.482 | 0.000 | 0.382 |
| 1598 | 2953 | Drought | 0.482 | 0.000 | 0.382 |
| 1606 | 2960 | Drought | 0.481 | 0.348 | 0.569 |
| 1607 | 2961 | Drought | 0.481 | 0.256 | 0.367 |
| 1608 | 2962 | Drought | 0.480 | 0.368 | 0.505 |
| 1609 | 2963 | Drought | 0.480 | 0.367 | 0.281 |
| 1610 | 2964 | Drought | 0.480 | 0.255 | 0.482 |
| 1614 | 2968 | Drought | 0.479 | 0.272 | 0.343 |
| 1616 | 2970 | Drought | 0.479 | 0.175 | 0.497 |
| 1617 | 2971 | Drought | 0.479 | 0.000 | 0.522 |
| 1618 | 2972 | Drought | 0.479 | 0.440 | 0.451 |
| 1619 | 2973 | Drought | 0.478 | 0.343 | 0.479 |
| 1620 | 2974 | Drought | 0.478 | 0.000 | 0.421 |
| 1622 | 2976 | Drought | 0.477 | 0.475 | 0.363 |
| 1623 | 2977 | Drought | 0.477 | 0.357 | 0.588 |
| 1624 | 2978 | Drought | 0.477 | 0.407 | 0.537 |
| 1625 | 2979 | Drought | 0.477 | 0.389 | 0.409 |
| 1626 | 2980 | Drought | 0.476 | 0.280 | 0.555 |
| 1627 | 2981 | Drought | 0.476 | 0.410 | 0.543 |
| 1628 | 2982 | Drought | 0.475 | 0.301 | 0.462 |
| 1630 | 2984 | Drought | 0.474 | 0.248 | 0.468 |
| 1631 | 2985 | Drought | 0.474 | 0.210 | 0.309 |
| 1632 | 2986 | Drought | 0.474 | 0.183 | 0.396 |
| 1633 | 2987 | Drought | 0.474 | 0.000 | 0.527 |
| 1634 | 2988 | Drought | 0.474 | 0.000 | 0.497 |
| 1635 | 2989 | Drought | 0.474 | 0.000 | 0.497 |
| 1636 | 2990 | Drought | 0.474 | 0.470 | 0.444 |
| 1637 | 2991 | Drought | 0.473 | 0.000 | 0.557 |
| 1638 | 2992 | Drought | 0.473 | 0.000 | 0.000 |
| 1639 | 2993 | Drought | 0.472 | 0.000 | 0.474 |
| 1640 | 2994 | Drought | 0.472 | 0.000 | 0.308 |
| 1644 | 2998 | Drought | 0.471 | 0.228 | 0.396 |
| 1645 | N.A. | Drought | 0.471 | 0.419 | 0.383 |
| 1646 | 2999 | Drought | 0.471 | 0.167 | 0.439 |
| 1647 | 3000 | Drought | 0.471 | 0.228 | 0.404 |
| 1648 | 3001 | Drought | 0.470 | 0.272 | 0.345 |
| 1649 | 3002 | Drought | 0.470 | 0.098 | 0.432 |
| 1650 | 3003 | Drought | 0.470 | 0.000 | 0.540 |
| 1651 | 3004 | Drought | 0.470 | 0.301 | 0.508 |
| 1652 | 3005 | Drought | 0.470 | 0.248 | 0.468 |
| 1653 | 3006 | Drought | 0.469 | 0.334 | 0.526 |
| 1654 | 3007 | Drought | 0.469 | 0.387 | 0.542 |
| 1656 | 3009 | Drought | 0.469 | 0.415 | 0.475 |
| 1657 | 3010 | Drought | 0.468 | 0.000 | 0.408 |
| 1658 | 3011 | Drought | 0.468 | 0.426 | 0.395 |
| 1659 | 3012 | Drought | 0.468 | 0.000 | 0.399 |
| 1660 | 3013 | Drought | 0.468 | 0.293 | 0.535 |
| 1661 | 3014 | Drought | 0.468 | 0.339 | 0.543 |
| 1663 | 3016 | Drought | 0.468 | 0.418 | 0.389 |
| 1665 | 3018 | Drought | 0.467 | 0.122 | 0.381 |
| 1666 | 3019 | Drought | 0.467 | 0.000 | 0.000 |
| 1667 | 3019 | Drought | 0.467 | 0.000 | 0.000 |
| 1668 | 3020 | Drought | 0.467 | 0.248 | 0.483 |
| 1669 | 3021 | Drought | 0.467 | 0.248 | 0.483 |
| 1670 | 3022 | Drought | 0.467 | 0.248 | 0.483 |
| 1671 | 3023 | Drought | 0.467 | 0.167 | 0.353 |
| 1672 | 3024 | Drought | 0.466 | 0.247 | 0.540 |
| 1673 | 3025 | Drought | 0.466 | 0.098 | 0.432 |
| 1674 | 3026 | Drought | 0.465 | 0.280 | 0.442 |
| 1675 | 3027 | Drought | 0.465 | 0.000 | 0.423 |
| 1676 | 3028 | Drought | 0.465 | 0.167 | 0.353 |
| 1677 | 3029 | Drought | 0.465 | 0.167 | 0.353 |
| 1678 | 3030 | Drought | 0.465 | 0.167 | 0.353 |
| 1679 | 3031 | Drought | 0.465 | 0.159 | 0.319 |
| 1681 | 3033 | Drought | 0.465 | 0.159 | 0.497 |
| 1682 | 3034 | Drought | 0.465 | 0.098 | 0.432 |
| 1686 | 3038 | Drought | 0.464 | 0.168 | 0.542 |
| 1687 | 3039 | Drought | 0.463 | 0.280 | 0.555 |
| 1688 | 3040 | Drought | 0.463 | 0.247 | 0.540 |
| 1689 | 3041 | Drought | 0.463 | 0.000 | 0.386 |
| 1690 | 3042 | Drought | 0.463 | 0.298 | 0.000 |
| 1691 | 3043 | Drought | 0.463 | 0.407 | 0.554 |
| 1692 | 3044 | Drought | 0.463 | 0.407 | 0.554 |
| 1693 | 3045 | Drought | 0.463 | 0.000 | 0.396 |
| 1694 | 3046 | Drought | 0.463 | 0.441 | 0.499 |
| 1695 | 3047 | Drought | 0.463 | 0.301 | 0.571 |
| 1696 | 3048 | Drought | 0.463 | 0.000 | 0.000 |
| 1697 | 3049 | Drought | 0.462 | 0.345 | 0.404 |
| 1698 | 3050 | Drought | 0.462 | 0.301 | 0.505 |
| 1699 | 3051 | Drought | 0.462 | 0.098 | 0.355 |
| 1700 | 3052 | Drought | 0.462 | 0.000 | 0.000 |
| 1701 | 3053 | Drought | 0.462 | 0.336 | 0.391 |
| 1702 | N.A. | Drought | 0.461 | 0.168 | 0.542 |
| 1705 | 3056 | Drought | 0.461 | 0.475 | 0.421 |
| 1706 | 3057 | Drought | 0.461 | 0.000 | 0.482 |
| 1708 | 3059 | Drought | 0.460 | 0.247 | 0.540 |
| 1149 | 2518 | Yield | 0.407 | 0.385 | 0.868 |
| 1152 | 2521 | Yield | 0.458 | 0.459 | 0.777 |
| 1156 | 2525 | Yield | 0.346 | 0.159 | 0.770 |
| 1159 | 2528 | Yield | 0.424 | 0.445 | 0.699 |
| 1162 | 2531 | Yield | 0.363 | 0.339 | 0.668 |
| 1163 | 2532 | Yield | 0.429 | 0.212 | 0.824 |
| 1165 | 2534 | Yield | 0.284 | 0.247 | 0.684 |
| 1170 | 2539 | Yield | 0.457 | 0.363 | 0.719 |
| 1171 | 2540 | Yield | 0.218 | 0.100 | 0.804 |
| 1172 | 2541 | Yield | 0.214 | 0.100 | 0.697 |
| 1174 | 2543 | Yield | 0.207 | 0.273 | 0.771 |
| 1181 | 2550 | Yield | 0.434 | 0.442 | 0.675 |
| 1182 | 2551 | Yield | 0.370 | 0.455 | 0.653 |
| 1186 | 2555 | Yield | 0.433 | 0.212 | 0.853 |
| 1187 | 2556 | Yield | 0.286 | 0.431 | 0.684 |
| 1189 | 2558 | Yield | 0.294 | 0.212 | 0.652 |
| 1191 | 2560 | Yield | 0.198 | 0.184 | 0.815 |
| 1193 | 2562 | Yield | 0.235 | 0.295 | 0.658 |
| 1196 | 2565 | Yield | 0.219 | 0.482 | 0.681 |
| 1197 | 2566 | Yield | 0.119 | 0.309 | 0.960 |
| 1200 | 2569 | Yield | 0.427 | 0.284 | 0.775 |
| 1203 | 2572 | Yield | 0.141 | 0.247 | 0.840 |
| 1204 | 2573 | Yield | 0.292 | 0.401 | 0.696 |
| 1210 | 2579 | Yield | 0.306 | 0.212 | 0.775 |
| 1211 | 2580 | Yield | 0.410 | 0.122 | 0.697 |
| 1212 | 2581 | Yield | 0.302 | 0.420 | 0.733 |
| 1214 | 2583 | Yield | 0.264 | 0.388 | 0.724 |
| 1215 | 2584 | Yield | 0.423 | 0.098 | 0.810 |
| 1217 | 2586 | Yield | 0.193 | 0.000 | 0.730 |
| 1219 | 2588 | Yield | 0.294 | 0.309 | 0.762 |
| 1225 | 2594 | Yield | 0.382 | 0.469 | 0.645 |
| 1226 | 2595 | Yield | 0.444 | 0.276 | 0.676 |
| 1229 | 2598 | Yield | 0.389 | 0.376 | 0.743 |
| 1230 | 2599 | Yield | 0.337 | 0.239 | 0.688 |
| 1235 | 2604 | Yield | 0.305 | 0.287 | 0.663 |
| 1241 | 2610 | Yield | 0.338 | 0.212 | 0.647 |
| 1242 | 2611 | Yield | 0.071 | 0.100 | 0.748 |
| 1245 | 2614 | Yield | 0.384 | 0.427 | 0.669 |
| 1248 | 2617 | Yield | 0.433 | 0.000 | 0.715 |
| 1250 | 2619 | Yield | 0.253 | 0.199 | 0.653 |
| 1342 | N.A. | Yield | 0.371 | 0.122 | 0.660 |
| 1342 | N.A. | Yield | 0.318 | 0.000 | 0.648 |
| 1342 | N.A. | Yield | 0.306 | 0.338 | 0.705 |
| 1342 | N.A. | Yield | 0.282 | 0.173 | 0.723 |
| 1342 | N.A. | Yield | 0.273 | 0.198 | 0.680 |
| 1342 | N.A. | Yield | 0.047 | 0.264 | 0.681 |
| 1342 | N.A. | Yield | 0.000 | 0.000 | 0.672 |
| 1702 | N.A. | Yield | 0.273 | 0.198 | 0.680 |
| 1702 | N.A. | Yield | 0.000 | 0.000 | 0.684 |
| 1709 | 3060 | Yield | 0.460 | 0.339 | 0.727 |
| 1712 | 3063 | Yield | 0.453 | 0.365 | 0.723 |
| 1713 | 3064 | Yield | 0.453 | 0.280 | 0.663 |
| 1714 | 3065 | Yield | 0.453 | 0.280 | 0.705 |
| 1718 | 3069 | Yield | 0.450 | 0.239 | 0.672 |
| 1719 | 3070 | Yield | 0.449 | 0.278 | 0.723 |
| 1723 | 3074 | Yield | 0.447 | 0.000 | 0.645 |
| 1724 | 3075 | Yield | 0.446 | 0.272 | 0.754 |
| 1725 | 3076 | Yield | 0.446 | 0.000 | 0.744 |
| 1729 | 3080 | Yield | 0.439 | 0.195 | 0.816 |
| 1730 | 3081 | Yield | 0.439 | 0.301 | 0.658 |
| 1732 | 3083 | Yield | 0.438 | 0.407 | 0.687 |
| 1735 | 3086 | Yield | 0.437 | 0.000 | 0.770 |
| 1736 | 3087 | Yield | 0.435 | 0.000 | 0.686 |
| 1743 | 3093 | Yield | 0.432 | 0.441 | 0.701 |
| 1744 | 3094 | Yield | 0.431 | 0.000 | 0.788 |
| 1746 | 3096 | Yield | 0.430 | 0.000 | 0.686 |
| 1747 | 3097 | Yield | 0.429 | 0.433 | 0.742 |
| 1748 | 3098 | Yield | 0.428 | 0.000 | 0.690 |
| 1749 | 3099 | Yield | 0.428 | 0.228 | 0.690 |
| 1750 | 3100 | Yield | 0.428 | 0.000 | 0.643 |
| 1751 | 3101 | Yield | 0.428 | 0.000 | 0.657 |
| 1754 | 3104 | Yield | 0.424 | 0.228 | 0.642 |
| 1755 | 3105 | Yield | 0.424 | 0.348 | 0.658 |
| 1756 | 3106 | Yield | 0.424 | 0.000 | 0.647 |
| 1758 | 3108 | Yield | 0.422 | 0.320 | 0.709 |
| 1762 | 3112 | Yield | 0.420 | 0.098 | 0.688 |
| 1768 | 3118 | Yield | 0.418 | 0.000 | 0.691 |
| 1769 | 3119 | Yield | 0.416 | 0.000 | 0.647 |
| 1770 | 3120 | Yield | 0.416 | 0.000 | 0.647 |
| 1772 | 3122 | Yield | 0.414 | 0.383 | 0.646 |
| 1773 | 3123 | Yield | 0.413 | 0.122 | 0.664 |
| 1774 | 3124 | Yield | 0.409 | 0.145 | 1.000 |
| 1776 | 3126 | Yield | 0.406 | 0.233 | 0.654 |
| 1779 | 3129 | Yield | 0.404 | 0.199 | 0.645 |
| 1780 | 3130 | Yield | 0.402 | 0.488 | 0.689 |
| 1782 | 3132 | Yield | 0.400 | 0.199 | 0.646 |
| 1787 | 3137 | Yield | 0.397 | 0.000 | 0.667 |
| 1788 | 3138 | Yield | 0.397 | 0.000 | 0.667 |
| 1789 | 3139 | Yield | 0.397 | 0.000 | 0.739 |
| 1790 | 3140 | Yield | 0.393 | 0.000 | 0.650 |
| 1791 | 3141 | Yield | 0.389 | 0.000 | 0.666 |
| 1792 | 3142 | Yield | 0.389 | 0.000 | 0.650 |
| 1793 | 3143 | Yield | 0.389 | 0.000 | 0.650 |
| 1794 | 3144 | Yield | 0.389 | 0.000 | 0.732 |
| 1796 | 3118 | Yield | 0.388 | 0.199 | 0.646 |
| 1797 | 3146 | Yield | 0.387 | 0.278 | 0.724 |
| 1798 | 3147 | Yield | 0.387 | 0.488 | 0.689 |
| 1799 | 3148 | Yield | 0.387 | 0.488 | 0.673 |
| 1800 | 3149 | Yield | 0.387 | 0.390 | 0.730 |
| 1801 | 3150 | Yield | 0.387 | 0.359 | 0.730 |
| 1802 | 3151 | Yield | 0.387 | 0.359 | 0.730 |
| 1803 | 3152 | Yield | 0.386 | 0.485 | 0.704 |
| 1804 | 3153 | Yield | 0.386 | 0.122 | 0.643 |
| 1805 | 3154 | Yield | 0.386 | 0.456 | 0.676 |
| 1806 | 3155 | Yield | 0.385 | 0.325 | 0.658 |
| 1811 | 3160 | Yield | 0.382 | 0.000 | 0.729 |
| 1816 | 3165 | Yield | 0.377 | 0.299 | 0.663 |
| 1817 | 3166 | Yield | 0.377 | 0.000 | 0.750 |
| 1818 | 3167 | Yield | 0.377 | 0.122 | 0.660 |
| 1819 | 3168 | Yield | 0.376 | 0.198 | 0.660 |
| 1821 | 3170 | Yield | 0.375 | 0.361 | 0.671 |
| 1822 | 3171 | Yield | 0.375 | 0.369 | 0.689 |
| 1824 | 3173 | Yield | 0.374 | 0.000 | 0.769 |
| 1828 | 3177 | Yield | 0.371 | 0.122 | 0.680 |
| 1829 | 3178 | Yield | 0.371 | 0.122 | 0.660 |
| 1830 | N.A. | Yield | 0.371 | 0.122 | 0.660 |
| 1831 | 3179 | Yield | 0.370 | 0.394 | 0.650 |
| 1835 | 3183 | Yield | 0.368 | 0.442 | 0.643 |
| 1837 | 3185 | Yield | 0.366 | 0.449 | 0.676 |
| 1838 | 3186 | Yield | 0.366 | 0.000 | 0.658 |
| 1839 | 3187 | Yield | 0.365 | 0.000 | 0.648 |
| 1840 | 3188 | Yield | 0.364 | 0.433 | 0.728 |
| 1841 | 3189 | Yield | 0.364 | 0.279 | 0.655 |
| 1846 | 3194 | Yield | 0.361 | 0.247 | 0.722 |
| 1849 | 3197 | Yield | 0.359 | 0.000 | 0.655 |
| 1850 | 3198 | Yield | 0.359 | 0.000 | 0.742 |
| 1851 | 3199 | Yield | 0.359 | 0.287 | 0.663 |
| 1853 | 3201 | Yield | 0.356 | 0.352 | 0.654 |
| 1854 | 3202 | Yield | 0.356 | 0.000 | 0.643 |
| 1855 | 3203 | Yield | 0.355 | 0.098 | 0.662 |
| 1856 | 3204 | Yield | 0.355 | 0.278 | 1.000 |
| 1864 | 3212 | Yield | 0.355 | 0.489 | 0.732 |
| 1865 | 3213 | Yield | 0.355 | 0.000 | 0.730 |
| 1869 | 3217 | Yield | 0.353 | 0.417 | 0.649 |
| 1870 | 3218 | Yield | 0.353 | 0.000 | 0.658 |
| 1875 | 3223 | Yield | 0.351 | 0.279 | 0.658 |
| 1876 | 3224 | Yield | 0.351 | 0.279 | 0.650 |
| 1877 | 3225 | Yield | 0.350 | 0.000 | 0.724 |
| 1878 | 3226 | Yield | 0.349 | 0.000 | 0.730 |
| 1879 | 3227 | Yield | 0.349 | 0.000 | 0.679 |
| 1883 | 3231 | Yield | 0.345 | 0.247 | 0.767 |
| 1885 | 3233 | Yield | 0.345 | 0.000 | 0.773 |
| 1886 | 3234 | Yield | 0.345 | 0.000 | 0.649 |
| 1887 | N.A. | Yield | 0.345 | 0.000 | 0.649 |
| 1891 | 3238 | Yield | 0.345 | 0.000 | 0.755 |
| 1900 | 3245 | Yield | 0.344 | 0.000 | 0.666 |
| 1902 | 3247 | Yield | 0.342 | 0.255 | 0.690 |
| 1904 | 3249 | Yield | 0.339 | 0.448 | 0.731 |
| 1905 | 3250 | Yield | 0.338 | 0.233 | 0.705 |
| 1906 | 3251 | Yield | 0.338 | 0.168 | 0.704 |
| 1907 | 3252 | Yield | 0.338 | 0.233 | 0.705 |
| 1908 | 3253 | Yield | 0.338 | 0.122 | 0.649 |
| 1909 | 3254 | Yield | 0.337 | 0.278 | 0.655 |
| 1910 | 3255 | Yield | 0.337 | 0.412 | 0.727 |
| 1912 | 3257 | Yield | 0.336 | 0.381 | 1.000 |
| 1917 | 3262 | Yield | 0.334 | 0.122 | 0.642 |
| 1918 | 3263 | Yield | 0.333 | 0.122 | 0.650 |
| 1919 | 3264 | Yield | 0.333 | 0.339 | 0.648 |
| 1920 | 3265 | Yield | 0.332 | 0.173 | 0.692 |
| 1920 | 3265 | Yield | 0.000 | 0.307 | 0.661 |
| 1921 | 3266 | Yield | 0.332 | 0.173 | 0.692 |
| 1922 | 3267 | Yield | 0.332 | 0.481 | 0.731 |
| 1925 | 3270 | Yield | 0.331 | 0.173 | 0.687 |
| 1927 | 3272 | Yield | 0.327 | 0.122 | 0.700 |
| 1928 | 3273 | Yield | 0.327 | 0.122 | 0.690 |
| 1932 | 3277 | Yield | 0.325 | 0.351 | 0.730 |
| 1934 | 3279 | Yield | 0.324 | 0.221 | 0.677 |
| 1935 | 3280 | Yield | 0.323 | 0.287 | 0.845 |
| 1938 | 3283 | Yield | 0.274 | 0.000 | 0.670 |
| 1939 | 3284 | Yield | 0.274 | 0.000 | 0.670 |
| 1943 | 3288 | Yield | 0.321 | 0.460 | 0.662 |
| 1944 | 3289 | Yield | 0.321 | 0.278 | 0.736 |
| 1946 | 3291 | Yield | 0.320 | 0.000 | 0.753 |
| 1947 | 3292 | Yield | 0.320 | 0.352 | 0.673 |
| 1948 | N.A. | Yield | 0.318 | 0.000 | 0.648 |
| 1950 | 3294 | Yield | 0.316 | 0.301 | 0.703 |
| 1951 | 3295 | Yield | 0.316 | 0.301 | 0.703 |
| 1953 | 3297 | Yield | 0.315 | 0.000 | 0.647 |
| 1957 | 3301 | Yield | 0.313 | 0.390 | 0.690 |
| 1959 | N.A. | Yield | 0.311 | 0.000 | 0.765 |
| 1960 | 3303 | Yield | 0.311 | 0.265 | 0.649 |
| 1962 | 3305 | Yield | 0.310 | 0.325 | 0.645 |
| 1963 | 3306 | Yield | 0.310 | 0.000 | 0.729 |
| 1964 | 3307 | Yield | 0.310 | 0.000 | 0.681 |
| 1965 | 3308 | Yield | 0.309 | 0.322 | 0.759 |
| 1967 | 3310 | Yield | 0.306 | 0.338 | 0.705 |
| 1968 | N.A. | Yield | 0.306 | 0.338 | 0.705 |
| 1972 | 3313 | Yield | 0.306 | 0.239 | 0.651 |
| 1973 | 3314 | Yield | 0.305 | 0.287 | 0.653 |
| 1974 | 3315 | Yield | 0.305 | 0.145 | 0.663 |
| 1975 | 3316 | Yield | 0.305 | 0.481 | 0.707 |
| 1984 | 3323 | Yield | 0.305 | 0.000 | 0.670 |
| 1985 | 3324 | Yield | 0.305 | 0.272 | 1.000 |
| 1986 | 3325 | Yield | 0.305 | 0.272 | 0.649 |
| 1987 | 3326 | Yield | 0.305 | 0.272 | 0.704 |
| 1988 | 3327 | Yield | 0.304 | 0.420 | 0.665 |
| 1989 | 3328 | Yield | 0.303 | 0.252 | 0.786 |
| 1992 | 3331 | Yield | 0.300 | 0.000 | 0.702 |
| 1993 | 3332 | Yield | 0.300 | 0.239 | 0.724 |
| 1994 | 3333 | Yield | 0.300 | 0.239 | 0.705 |
| 1995 | 3334 | Yield | 0.300 | 0.239 | 0.670 |
| 1996 | 3335 | Yield | 0.300 | 0.382 | 0.730 |
| 1997 | 3336 | Yield | 0.300 | 0.122 | 0.697 |
| 1998 | 3337 | Yield | 0.300 | 0.122 | 0.652 |
| 2008 | 3346 | Yield | 0.296 | 0.000 | 0.769 |
| 2010 | 3348 | Yield | 0.295 | 0.229 | 0.725 |
| 2011 | 3349 | Yield | 0.295 | 0.287 | 0.745 |
| 2012 | 3350 | Yield | 0.295 | 0.287 | 0.727 |
| 2013 | 3351 | Yield | 0.294 | 0.290 | 0.693 |
| 2015 | 3353 | Yield | 0.292 | 0.122 | 0.647 |
| 2016 | 3354 | Yield | 0.292 | 0.272 | 0.743 |
| 2017 | 3355 | Yield | 0.292 | 0.272 | 0.692 |
| 2018 | 3356 | Yield | 0.292 | 0.272 | 0.723 |
| 2019 | 3357 | Yield | 0.292 | 0.272 | 0.723 |
| 2020 | 3358 | Yield | 0.290 | 0.200 | 0.858 |
| 2021 | 3359 | Yield | 0.289 | 0.352 | 0.654 |
| 2022 | 3360 | Yield | 0.289 | 0.352 | 0.654 |
| 2024 | 3362 | Yield | 0.287 | 0.000 | 0.655 |
| 2034 | 3371 | Yield | 0.287 | 0.167 | 0.729 |
| 2036 | 3373 | Yield | 0.287 | 0.387 | 0.656 |
| 2037 | 3374 | Yield | 0.287 | 0.183 | 0.844 |
| 2039 | 3376 | Yield | 0.286 | 0.167 | 0.681 |
| 2040 | 3377 | Yield | 0.285 | 0.000 | 0.652 |
| 2043 | 3380 | Yield | 0.285 | 0.000 | 0.651 |
| 2044 | 3381 | Yield | 0.285 | 0.000 | 0.657 |
| 2045 | 3382 | Yield | 0.285 | 0.000 | 0.656 |
| 2046 | 3383 | Yield | 0.285 | 0.171 | 0.668 |
| 2047 | 3384 | Yield | 0.285 | 0.171 | 0.796 |
| 2048 | 3385 | Yield | 0.285 | 0.173 | 0.697 |
| 2049 | 3386 | Yield | 0.284 | 0.319 | 0.673 |
| 2050 | 3387 | Yield | 0.284 | 0.319 | 0.673 |
| 2051 | 3388 | Yield | 0.284 | 0.256 | 0.661 |
| 2052 | 3052 | Yield | 0.284 | 0.256 | 0.772 |
| 2054 | 3390 | Yield | 0.284 | 0.256 | 0.660 |
| 2055 | 3391 | Yield | 0.282 | 0.390 | 0.672 |
| 2056 | 3392 | Yield | 0.282 | 0.390 | 0.928 |
| 2057 | 3393 | Yield | 0.282 | 0.361 | 0.651 |
| 2058 | 3394 | Yield | 0.282 | 0.173 | 0.696 |
| 2059 | 3395 | Yield | 0.281 | 0.420 | 0.669 |
| 2060 | 3396 | Yield | 0.281 | 0.420 | 0.665 |
| 2061 | 3397 | Yield | 0.281 | 0.366 | 0.731 |
| 2062 | 3398 | Yield | 0.281 | 0.366 | 0.709 |
| 2063 | 3399 | Yield | 0.281 | 0.394 | 0.770 |
| 2064 | N.A. | Yield | 0.280 | 0.239 | 0.693 |
| 2065 | 3400 | Yield | 0.278 | 0.352 | 0.773 |
| 2069 | 3404 | Yield | 0.276 | 0.167 | 0.770 |
| 2070 | 3405 | Yield | 0.276 | 0.167 | 0.697 |
| 2071 | 3406 | Yield | 0.276 | 0.167 | 0.720 |
| 2072 | 3407 | Yield | 0.276 | 0.167 | 0.715 |
| 2073 | 3408 | Yield | 0.274 | 0.281 | 0.672 |
| 2075 | 3410 | Yield | 0.274 | 0.000 | 0.734 |
| 2076 | 3411 | Yield | 0.274 | 0.325 | 0.678 |
| 2077 | 3412 | Yield | 0.274 | 0.000 | 0.694 |
| 2078 | 3413 | Yield | 0.273 | 0.339 | 0.659 |
| 2079 | 3414 | Yield | 0.273 | 0.000 | 0.845 |
| 2080 | 3415 | Yield | 0.273 | 0.256 | 0.696 |
| 2085 | 3420 | Yield | 0.273 | 0.198 | 0.731 |
| 2086 | 3421 | Yield | 0.273 | 0.198 | 0.731 |
| 2087 | 3422 | Yield | 0.271 | 0.229 | 0.648 |
| 2089 | 3424 | Yield | 0.270 | 0.000 | 0.696 |
| 2090 | 3425 | Yield | 0.270 | 0.199 | 0.646 |
| 2095 | 3430 | Yield | 0.266 | 0.287 | 0.651 |
| 2096 | 3431 | Yield | 0.266 | 0.287 | 0.651 |
| 2097 | N.A. | Yield | 0.261 | 0.420 | 0.669 |
| 2098 | N.A. | Yield | 0.261 | 0.420 | 0.669 |
| 2102 | 3435 | Yield | 0.259 | 0.000 | 0.739 |
| 2103 | 3436 | Yield | 0.259 | 0.436 | 0.653 |
| 2104 | 3437 | Yield | 0.259 | 0.000 | 0.687 |
| 2106 | 3439 | Yield | 0.257 | 0.247 | 0.722 |
| 2107 | 3440 | Yield | 0.257 | 0.301 | 0.668 |
| 2113 | 3444 | Yield | 0.253 | 0.365 | 0.921 |
| 2114 | 3445 | Yield | 0.253 | 0.199 | 0.725 |
| 2115 | 3446 | Yield | 0.253 | 0.199 | 0.652 |
| 2116 | 3447 | Yield | 0.252 | 0.000 | 0.690 |
| 2121 | 3452 | Yield | 0.248 | 0.239 | 0.693 |
| 2122 | 3453 | Yield | 0.246 | 0.000 | 0.660 |
| 2123 | 3454 | Yield | 0.246 | 0.000 | 0.669 |
| 2124 | 3455 | Yield | 0.246 | 0.000 | 0.687 |
| 2125 | 3456 | Yield | 0.246 | 0.000 | 0.687 |
| 2128 | 3459 | Yield | 0.245 | 0.473 | 0.735 |
| 2129 | 3460 | Yield | 0.245 | 0.473 | 0.664 |
| 2131 | 3462 | Yield | 0.244 | 0.331 | 0.693 |
| 2132 | 3463 | Yield | 0.243 | 0.000 | 0.655 |
| 2133 | 3464 | Yield | 0.242 | 0.420 | 0.784 |
| 2137 | 3468 | Yield | 0.237 | 0.000 | 0.651 |
| 2138 | 3469 | Yield | 0.237 | 0.000 | 0.651 |
| 2139 | 3470 | Yield | 0.237 | 0.000 | 0.747 |
| 2140 | 3471 | Yield | 0.237 | 0.000 | 0.747 |
| 2141 | 3472 | Yield | 0.236 | 0.363 | 0.648 |
| 2143 | 3474 | Yield | 0.231 | 0.122 | 0.677 |
| 2144 | 3475 | Yield | 0.228 | 0.000 | 0.699 |
| 2146 | N.A. | Yield | 0.224 | 0.286 | 0.676 |
| 2147 | 3477 | Yield | 0.220 | 0.274 | 0.705 |
| 2148 | 3478 | Yield | 0.220 | 0.274 | 0.650 |
| 2149 | 3479 | Yield | 0.220 | 0.274 | 0.648 |
| 2150 | 3480 | Yield | 0.220 | 0.274 | 0.648 |
| 2153 | 3483 | Yield | 0.220 | 0.293 | 0.723 |
| 2154 | 3484 | Yield | 0.220 | 0.293 | 0.666 |
| 2155 | 3485 | Yield | 0.220 | 0.293 | 0.766 |
| 2156 | 3486 | Yield | 0.219 | 0.084 | 1.000 |
| 2158 | 3488 | Yield | 0.218 | 0.256 | 0.654 |
| 2159 | 3489 | Yield | 0.218 | 0.334 | 0.721 |
| 2160 | 3490 | Yield | 0.215 | 0.318 | 0.642 |
| 2161 | 3491 | Yield | 0.213 | 0.000 | 0.744 |
| 2164 | 3494 | Yield | 0.211 | 0.000 | 0.724 |
| 2165 | 3495 | Yield | 0.210 | 0.000 | 0.710 |
| 2167 | 3497 | Yield | 0.208 | 0.199 | 0.645 |
| 2168 | N.A. | Yield | 0.208 | 0.199 | 0.645 |
| 2169 | 3498 | Yield | 0.205 | 0.378 | 0.722 |
| 2171 | 3500 | Yield | 0.205 | 0.173 | 0.680 |
| 2172 | 3501 | Yield | 0.205 | 0.173 | 0.679 |
| 2173 | 3502 | Yield | 0.205 | 0.173 | 0.846 |
| 2180 | 3509 | Yield | 0.204 | 0.000 | 0.655 |
| 2181 | 3510 | Yield | 0.204 | 0.000 | 0.848 |
| 2182 | 3511 | Yield | 0.204 | 0.000 | 0.655 |
| 2183 | 3512 | Yield | 0.201 | 0.301 | 0.668 |
| 2184 | 3513 | Yield | 0.200 | 0.297 | 0.709 |
| 2185 | 3514 | Yield | 0.199 | 0.334 | 0.721 |
| 2186 | 3515 | Yield | 0.198 | 0.180 | 0.844 |
| 2187 | 3516 | Yield | 0.196 | 0.462 | 0.670 |
| 2188 | 3517 | Yield | 0.191 | 0.289 | 0.689 |
| 2190 | 3519 | Yield | 0.190 | 0.122 | 0.664 |
| 2191 | 3520 | Yield | 0.189 | 0.453 | 0.668 |
| 2201 | 3530 | Yield | 0.178 | 0.000 | 0.664 |
| 2203 | 3532 | Yield | 0.175 | 0.122 | 0.664 |
| 2204 | 3533 | Yield | 0.173 | 0.411 | 0.731 |
| 2206 | 3535 | Yield | 0.173 | 0.452 | 0.661 |
| 2207 | 3536 | Yield | 0.171 | 0.335 | 0.701 |
| 2208 | N.A. | Yield | 0.166 | 0.000 | 0.776 |
| 2212 | 3540 | Yield | 0.160 | 0.000 | 0.847 |
| 2214 | 3542 | Yield | 0.159 | 0.437 | 0.659 |
| 2215 | 3543 | Yield | 0.157 | 0.221 | 0.658 |
| 2216 | 3544 | Yield | 0.157 | 0.122 | 0.664 |
| 2218 | 3546 | Yield | 0.153 | 0.000 | 0.729 |
| 2219 | 3547 | Yield | 0.153 | 0.331 | 0.707 |
| 2220 | 3548 | Yield | 0.152 | 0.000 | 0.679 |
| 2221 | 3549 | Yield | 0.150 | 0.278 | 0.765 |
| 2222 | 3550 | Yield | 0.150 | 0.385 | 0.675 |
| 2225 | 3553 | Yield | 0.146 | 0.000 | 0.672 |
| 2226 | 3554 | Yield | 0.146 | 0.000 | 0.672 |
| 2227 | 3555 | Yield | 0.146 | 0.000 | 0.674 |
| 2228 | 3556 | Yield | 0.146 | 0.428 | 0.749 |
| 2229 | 3557 | Yield | 0.146 | 0.274 | 0.667 |
| 2231 | 3559 | Yield | 0.143 | 0.274 | 0.667 |
| 2232 | 3560 | Yield | 0.141 | 0.000 | 0.843 |
| 2233 | 3561 | Yield | 0.135 | 0.159 | 0.692 |
| 2234 | 3562 | Yield | 0.132 | 0.233 | 0.711 |
| 2235 | 3563 | Yield | 0.132 | 0.233 | 0.665 |
| 2237 | 3565 | Yield | 0.129 | 0.000 | 0.843 |
| 2239 | 3567 | Yield | 0.125 | 0.335 | 0.668 |
| 2242 | 3570 | Yield | 0.125 | 0.410 | 0.642 |
| 2244 | 3572 | Yield | 0.119 | 0.000 | 0.673 |
| 2252 | 3580 | Yield | 0.106 | 0.279 | 0.665 |
| 2255 | 3583 | Yield | 0.101 | 0.000 | 0.755 |
| 2256 | 3584 | Yield | 0.101 | 0.084 | 0.709 |
| 2257 | 3585 | Yield | 0.098 | 0.274 | 0.653 |
| 2258 | 3586 | Yield | 0.098 | 0.410 | 0.709 |
| 2259 | 3587 | Yield | 0.098 | 0.274 | 0.709 |
| 2260 | 3588 | Yield | 0.098 | 0.274 | 0.844 |
| 2261 | 3589 | Yield | 0.098 | 0.274 | 0.690 |
| 2263 | 3591 | Yield | 0.092 | 0.279 | 0.656 |
| 2264 | 3592 | Yield | 0.092 | 0.279 | 0.750 |
| 2265 | 3593 | Yield | 0.086 | 0.159 | 0.647 |
| 2266 | 3594 | Yield | 0.086 | 0.159 | 0.706 |
| 2267 | 3595 | Yield | 0.086 | 0.159 | 0.706 |
| 2268 | 3596 | Yield | 0.086 | 0.159 | 0.668 |
| 2269 | 3597 | Yield | 0.086 | 0.159 | 0.767 |
| 2271 | 3599 | Yield | 0.047 | 0.270 | 0.653 |
| 2272 | 3600 | Yield | 0.047 | 0.289 | 0.656 |
| 2274 | 3602 | Yield | 0.047 | 0.280 | 0.658 |
| 2277 | 3605 | Yield | 0.047 | 0.279 | 0.669 |
| 2278 | 3606 | Yield | 0.047 | 0.279 | 0.674 |
| 2280 | N.A. | Yield | 0.047 | 0.264 | 0.681 |
| 2281 | 3608 | Yield | 0.047 | 0.379 | 0.848 |
| 2282 | 3609 | Yield | 0.047 | 0.379 | 0.728 |
| 2285 | 3612 | Yield | 0.047 | 0.301 | 0.696 |
| 2288 | 3615 | Yield | 0.045 | 0.139 | 0.779 |
| 2297 | 3623 | Yield | 0.043 | 0.000 | 0.692 |
| 2298 | 3624 | Yield | 0.043 | 0.339 | 0.655 |
| 2302 | 3628 | Yield | 0.043 | 0.000 | 0.695 |
| 2304 | 3630 | Yield | 0.021 | 0.098 | 0.694 |
| 2306 | 3632 | Yield | 0.021 | 0.171 | 0.675 |
| 2311 | 3637 | Yield | 0.013 | 0.000 | 0.733 |
| 2312 | 3638 | Yield | 0.013 | 0.210 | 0.659 |
| 2317 | 3643 | Yield | 0.000 | 0.000 | 0.642 |
| 2318 | 3644 | Yield | 0.000 | 0.167 | 0.660 |
| 2319 | 3645 | Yield | 0.000 | 0.228 | 0.649 |
| 2323 | 3649 | Yield | 0.000 | 0.000 | 0.687 |
| 2326 | 3652 | Yield | 0.000 | 0.000 | 0.694 |
| 2332 | 3658 | Yield | 0.000 | 0.000 | 1.000 |
| 2333 | 3659 | Yield | 0.000 | 0.000 | 0.655 |
| 2334 | 3660 | Yield | 0.000 | 0.000 | 0.655 |
| 2335 | 3661 | Yield | 0.000 | 0.000 | 0.642 |
| 2336 | 3662 | Yield | 0.000 | 0.453 | 0.656 |
| 2337 | 3663 | Yield | 0.000 | 0.453 | 0.708 |
| 2338 | 3664 | Yield | 0.000 | 0.173 | 0.845 |
| 2339 | 3665 | Yield | 0.000 | 0.000 | 0.785 |
| 2342 | 3668 | Yield | 0.000 | 0.000 | 0.670 |
| 2349 | 3503 | Yield | 0.000 | 0.098 | 0.666 |
| 2350 | 3675 | Yield | 0.000 | 0.000 | 0.647 |
| 2351 | 3676 | Yield | 0.000 | 0.000 | 0.656 |
| 2354 | 3679 | Yield | 0.000 | 0.000 | 0.708 |
| 2356 | 3681 | Yield | 0.000 | 0.000 | 0.642 |
| 2357 | 3682 | Yield | 0.000 | 0.228 | 0.642 |
| 2359 | 3684 | Yield | 0.000 | 0.331 | 0.662 |
| 2361 | 3686 | Yield | 0.000 | 0.000 | 0.642 |
| 2362 | 3687 | Yield | 0.000 | 0.167 | 0.653 |
| 2363 | 3688 | Yield | 0.000 | 0.167 | 0.657 |
| 2364 | 3689 | Yield | 0.000 | 0.167 | 0.660 |
| 2372 | 3697 | Yield | 0.000 | 0.000 | 0.720 |
| 2373 | N.A. | Yield | 0.000 | 0.000 | 0.642 |
| 2374 | 3698 | Yield | 0.000 | 0.339 | 0.745 |
| 2375 | 3699 | Yield | 0.000 | 0.339 | 0.843 |
| 2376 | 3700 | Yield | 0.000 | 0.000 | 0.705 |
| 2377 | 3701 | Yield | 0.000 | 0.000 | 0.678 |
| 2378 | N.A. | Yield | 0.000 | 0.000 | 0.678 |
| 2379 | N.A. | Yield | 0.000 | 0.000 | 0.678 |
| 2380 | 3702 | Yield | 0.000 | 0.229 | 0.756 |
| 2381 | 3703 | Yield | 0.000 | 0.229 | 0.693 |
| 2382 | 3704 | Yield | 0.000 | 0.485 | 0.861 |
| 2383 | 3705 | Yield | 0.000 | 0.485 | 0.844 |
| 2386 | 3708 | Yield | 0.000 | 0.247 | 0.662 |
| 2387 | 3709 | Yield | 0.000 | 0.339 | 0.680 |
| 2388 | 3710 | Yield | 0.000 | 0.339 | 0.648 |
| 2389 | 3711 | Yield | 0.000 | 0.339 | 0.671 |
| 2390 | 3712 | Yield | 0.000 | 0.339 | 0.659 |
| 2391 | 3713 | Yield | 0.000 | 0.339 | 0.659 |
| 2392 | 3714 | Yield | 0.000 | 0.371 | 0.696 |
| 2396 | 3718 | Yield | 0.000 | 0.000 | 0.765 |
| 2397 | 3719 | Yield | 0.000 | 0.000 | 0.744 |
| 2398 | 3720 | Yield | 0.000 | 0.000 | 0.744 |
| 2399 | N.A. | Yield | 0.000 | 0.000 | 0.744 |
| 2400 | 3721 | Yield | 0.000 | 0.000 | 0.690 |
| 2401 | 3722 | Yield | 0.000 | 0.000 | 0.654 |
| 2402 | 3723 | Yield | 0.000 | 0.357 | 0.651 |
| 2403 | 3724 | Yield | 0.000 | 0.357 | 0.651 |
| 2404 | 3725 | Yield | 0.000 | 0.357 | 0.692 |
| 2405 | 3726 | Yield | 0.000 | 0.357 | 0.692 |
| 2406 | 3727 | Yield | 0.000 | 0.357 | 0.692 |
| 2407 | 3728 | Yield | 0.000 | 0.357 | 0.692 |
| 2408 | 3729 | Yield | 0.000 | 0.000 | 0.667 |
| 2409 | N.A. | Yield | 0.000 | 0.000 | 0.684 |
| 2410 | N.A. | Yield | 0.000 | 0.000 | 0.684 |
| 2411 | 3730 | Yield | 0.000 | 0.000 | 0.728 |
| 2426 | 3740 | Yield | 0.000 | 0.000 | 0.646 |
| 2428 | 3742 | Yield | 0.000 | 0.279 | 0.655 |
| 2434 | 3747 | Yield | 0.000 | 0.000 | 0.720 |
| 2435 | 3748 | Yield | 0.000 | 0.000 | 0.765 |
| 2436 | 3749 | Yield | 0.000 | 0.307 | 0.692 |
| 2437 | 3750 | Yield | 0.000 | 0.307 | 0.692 |
| 2438 | 3751 | Yield | 0.000 | 0.307 | 0.692 |
| 2439 | 3752 | Yield | 0.000 | 0.307 | 0.684 |
| 2440 | 3753 | Yield | 0.000 | 0.000 | 0.676 |
| 2441 | 3754 | Yield | 0.000 | 0.000 | 0.667 |
| 2445 | 3758 | Yield | 0.000 | 0.000 | 0.642 |
| 2447 | 3760 | Yield | 0.000 | 0.000 | 0.649 |
| 2448 | 3761 | Yield | 0.000 | 0.000 | 0.722 |
| 2449 | N.A. | Yield | 0.000 | 0.000 | 0.672 |
| 2450 | 3762 | Yield | 0.000 | 0.171 | 0.751 |
| 2451 | 3763 | Yield | 0.000 | 0.171 | 0.656 |
| 2452 | 3764 | Yield | 0.000 | 0.171 | 0.656 |
| 2453 | 3765 | Yield | 0.000 | 0.171 | 0.724 |
| 2458 | 3770 | Yield | 0.000 | 0.000 | 0.673 |
| 2459 | 3771 | Yield | 0.000 | 0.000 | 0.694 |
| 2460 | 3772 | Yield | 0.000 | 0.000 | 0.694 |
| 2461 | 3773 | Yield | 0.000 | 0.000 | 0.696 |
| 2462 | 3774 | Yield | 0.000 | 0.409 | 0.659 |
| 2463 | 3775 | Yield | 0.000 | 0.210 | 0.665 |
| 2464 | 3776 | Yield | 0.000 | 0.210 | 0.665 |
| 2465 | 3777 | Yield | 0.000 | 0.210 | 0.705 |
| 2466 | 3778 | Yield | 0.000 | 0.437 | 0.650 |
| 2467 | 3779 | Yield | 0.000 | 0.309 | 0.686 |
| 2469 | 3781 | Yield | 0.000 | 0.378 | 0.722 |
| 2477 | 3789 | Yield | 0.000 | 0.000 | 0.729 |
| 2478 | 3790 | Yield | 0.000 | 0.000 | 0.855 |
| 2479 | 3791 | Yield | 0.000 | 0.000 | 0.843 |
| 2480 | 3792 | Yield | 0.000 | 0.422 | 0.721 |
| 2481 | 3793 | Yield | 0.000 | 0.116 | 1.000 |
| 2482 | 3794 | Yield | 0.000 | 0.116 | 0.666 |
| 2483 | 3795 | Yield | 0.000 | 0.116 | 0.666 |
| 2484 | 3796 | Yield | 0.000 | 0.116 | 0.666 |
| 2485 | 3797 | Yield | 0.000 | 0.116 | 0.767 |
| 2486 | 3798 | Yield | 0.000 | 0.116 | 0.707 |
| 2487 | N.A. | Yield | 0.000 | 0.247 | 0.691 |
| 2488 | 3550 | Yield | 0.000 | 0.247 | 0.675 |
| 2489 | 3799 | Yield | 0.000 | 0.475 | 0.665 |
| 2490 | 3800 | Yield | 0.000 | 0.000 | 0.643 |
| 2491 | 3801 | Yield | 0.000 | 0.000 | 0.643 |

### EXAMPLES

The following are non-limiting examples intended to illustrate the various embodiments.

### Example 1

### Genome-Wide Survey and Identification of MicroRNAs, Pre-Cursor Genes and Targets

MicroRNAs (miRNAs) are small non-coding RNAs that serve as regulators of gene expression and diverse biological functions in plants. Maize genome sequences were analyzed for B73 inbred and source gene candidates were classified and their predicted target regulated genes. Databases were searched to identify miRNA precursor genes that have predicted hairpin structures and/or related to one or more of about 4,698 plant mature miRNAs from miRBase and other sources. Additional miRNA precursors were identified by aligning all predicted miRNA hairpin sequences in plants from miRBase to the B73 psuedomolecules sequences, yielding at least 8,535 putative miRNA loci.

Maize small RNA sequencing reads from a profiling experiment were used to filter out predicted miRNA precursor loci having less than 10 sequence reads support thereby classifying them as computationally predicted but unexpressed precursor candidates. A software tool was developed to fetch the exact mature miRNA sequences from the B73 genome based on the predicted miRNA gene coordinates and the reference mature miRNA sequences from miRBase. A total of 321 maize miRNAs precursors were obtained from miRBase, and retained for analysis even if some did not have 10 sequencing reads from the profiling experiment. After removing overlapping miRNA loci between the two sets, the resulting miRNA precursor set had a total of 1,512 miRNA gene loci corresponding to about 197 unique mature miRNA sequences (core miRNA sequences).

Following identification of the source miRNA genes, the next step was to identify and prioritize miRNA target genes. Following a comprehensive survey, identification and classification of miRNA source genes in maize using the miRBase resources and other tools, the predicted target genes for these miRNAs were identified using the program miRanda (Enright et al., (2005), Human MicroRNA Targets, PLoS Biol.:e264) for predicting the targets for all 197 unique miRNAs. A total of 192 out of 197 miRNAs were predicted to have targets in the maize genome, averaging 59 targets per miRNA, but ranging from 1 to 1510 (alignment score 160 and energy score −30). These predicted miRNA targets are likely to be enriched for functional partners with the miRNAs, for example, genes that are regulated by the miRNAs.

### Example 2

### miRNA to Target Anti-Correlation Analysis

Gene which are regulated by miRNAs are expected to exhibit an expression pattern that is anti-correlated to the miRNA. This anti-correlation of expression of a target gene is an indication that the identified miRNA is likely regulating that target gene. It is possible that some genes may be anti-correlated by coincidence may not represent a true target for regulation by the identified microRNA. One way to determine the anti-correlation relationship is to analyze the binding sites on the target gene that is suspected to be anti-correlated with the miRNA expression.

Experiments were performed to identify gene pairs of miRNAs and their possible targets. One of the approaches to identify the miRNAs and the targets was using anti-correlated gene expression for miRNAs and their candidate genes through separate microarray profiling experiments. By comparing the mRNA profiling results for different microarrays using the same biological samples, and spanning over several tissues, it was determined whether the expression of one or more miRNAs correlated with their candidate target genes through statistical tools. Significant correlations were identified that demonstrated decreasing candidate gene transcript levels while the expression levels of the microRNA candidates increased. Some of these gene pairs also bore sequence similarity of the putative miRNA binding site, a 21-mer, providing further support that these genes may represent a regulated unit, with the miRNA acting as the agent of regulation.

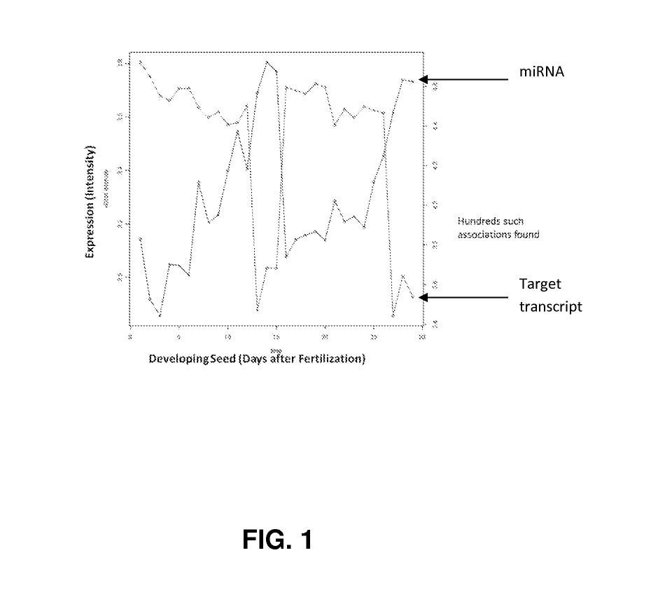

Empirical determination of miRNA targets was also performed. To empirically determine miRNA-mRNA counter-correlated pairs, 65 samples that were assayed with both the 105K mRNA microarray and the 44K miRNA microarray were examined. The 65 samples included 18 leaf samples from a circadian study, 18 immature ear samples from a circadian study and 29 kernel samples from a study examining transgenic zein knockdown expression. Only 42,758 probes from the mRNA array were considered to be expressed and used for the subsequent analysis. Correlation was determined by Pearson correlation coefficient and those mRNA-miRNA pairs that exhibited <(−0.9) were considered significant. An example of an anticorrelated gene pair from these experiments are shown in FIG. 1 The anticorrelation of the miRNA and the target gene (transcript) are indicated.

### Example 3

### Identification of Maize miRNA Sequences for Use in Agronomic Traits

The miRNA targets listed in Table 1 and whose sequences are provided herein to the sequence listing appended herein were analyzed for their significance to impacting one or more agronomic traits using bioinformatics tools. Results from these analyses were used to identify assign an agronomic parameter of importance to one or more of these gene targets as in Table 2. Drought, nitrogen and yield were chosen as three relevant agronomic traits and each target gene's relevance is listed in Table 2. For example, the same gene may appear for all three agronomic traits and some genes may fall under only of the selected traits. Relative trait values provided in Table 2 indicate the likelihood that a particular gene is regulated by a miRNA that impacts an agronomic trait of interest.

Gene networks were constructed from these gene relationships derived from bioinformatics analysis by linking genes to interaction and regulation partners, metabolic targets, trait component processes, and to other biologically relevant factors. A global gene network was also constructed based on all obtainable biologically relevant information, not limited to these three traits, creating a general or universal background network, against which to compare versus the three trait enriched networks. Relative trait values were developed and assigned to individual genes, based upon bioinformatics analysis. For cross-comparison of all three trait values, the values were all transformed to a 0-to-1 relative scale. For the miRNA target genes, these scores enable comparative analysis within a particular trait association, and across these agronomic traits.

### Example 4

### Gene Regulation with Transgenic MicroRNAs

One or more microRNA sequences listed in Table 1 and the sequence listing provided herein can be used to construct siRNA (small interfering RNA) vector or a vector that regulates genes in an equivalent manner. The genes may be operably controlled by a variety of plant-expressible promoter sequences to achieve broad or specific tissue-developmental or environmental response expression patterns. Maize plants, other crop plants, or model plants such as Arabidopsis can be transformed with the vector containing the miRNA hairpin construct or a microRNA precursor gene, and the transformants (e.g., at T0 or T1) can be evaluated for improved drought tolerance or NUE or yield increase (e.g., such as through a surrogate parameter such as photosynthetic activity, nutrient uptake, biomass increase).

When miRNA precursors are expressed, the expressed miRNA precursors are processed by the plants' resident microRNA processing apparatus and produce a mature miRNA sequence with regulatory function. The target genes of this miRNA will be expected to have reduced gene expression, transcript levels, or translation, resulting in reduced functional capacity of the target gene product. For target genes that are net negative regulators of agronomic trait performance, this reduction of their functional expression will lead to increased trait performance and agronomic gain. Some genes are involved in the evolved natural adaptive responses of plants to environmental stresses such as drought and nutrient deprivation, but in an agronomic setting these responses can negatively affect crop performance and yield. For example, some drought related genes contribute to a defensive slow-growing habit and physiology. With this miRNA targeting strategy, these genes can be selectively reduced in expression under these environmental conditions, enabling the plants to manage drought stress while maintaining a high yield capacity.

### Example 5

### Upregulating Plant miRNA Target Genes Through Down-Regulation of a miRNA Precursor Gene

Some agronomic traits are regulated at least in part by microRNAs, Some of these miRNA regulations are the result of long-evolved mechanisms to adapt to environmental stresses such as drought and nutrient limitations, such as nitrogen. The microRNA precursors may embody some of the tissue-developmental-environmental responsiveness for miRNA-based gene regulation. In situations where the target gene that may contribute to increased agronomic performance is being limited in net functional expression by a miRNA regulation, reduction (in site and location) in the expression of the microRNA precursor can result increased expression of the target gene and lead to increased agronomic trait performance. The reduction in the microRNA precursor expression may include targeting the miRNA expression by another siRNA construct, or by targeted mutagenesis, such as homing endonuclease-based site-directed changes that introduce functional changes in the expression and/or direct alteration of the core miRNA site.

### Example 6

### Use of miRNA Precursor Genes

The miRNA precursor genes can be upregulated through many ways—e.g., by expressing the precursor gene under the control of a plant expressible regulatory element or by upregulating the endogenous precursor gene through engineering a plant expressible regulatory element into the plant genome.

Similarly, the miRNA precursor gene loci can be mutagenized to either decrease or increase the expression of the precursor gene, e.g., by targeting the endogenous promoter element. miRNA genes can also serve as templates to construct artificial miRNA vector constructs to express an artificial miRNA transcript.

The precursor gene sequences can also be used as markers for marker-assisted breeding selection or to screen a population of maize plants for alleles of the precursor genes. For example, variations within the precursor sequences can result in SNPs that are used as markers or haplotypes for germplasm selection and breeding.

The miRNA sequences or the miRNA precursor gene sequences or the target gene sequences disclosed herein can be used as a template to design an artificial or a synthetic interfering RNA construct including an artificial miRNA or siRNA construct or synthetic polynucleotides encoding an interfering RNA thereof. As known in the art, these artificial nucleic acid sequences can contain one or more mismatches compared to the template and may also contain stabilizing nucleotide analogs for use as topical or other exogenous applications, where stability of nucleic acids are desirable.

### Example 7

### Use of Target Genes Disclosed in Tables 1 and 2

The target genes disclosed herein have been selected to contribute to one or more agronomic traits based on the identification of miRNAs and associated precursor genes. The target genes disclosed herein can be overexpressed constitutively, suppressed for example through RNA silencing/ The target genes can also be expressed as a synthetic version of the gene that is not directly targeted by an endogenous miRNA, thereby desensitizing the transgene copy from being subject to endogenous regulation. Desensitization can also be performed through mutagenesis for example to eliminate a potential miRNA binding site or altering the binding specificity to a closely related gene homolog. Any promoter/vector combination can be used with the target genes.

In addition, the target gene sequences can also be used as markers for marker-assisted breeding selection or to screen a population of maize plants for alleles of the target genes. For example, variations within the target gene sequences can result in SNPs that are used as markers or haplotypes for germplasm selection and breeding.

### Transformation of Plants

Described in this example are methods one may use for introduction of a polynucleotide or polypeptide into a plant cell.

### A. Maize Particle-Mediated DNA Delivery

A DNA construct can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473-497, with 0.25 M sorbitol). The embryos on the high-osmotic medium are used as the bombardment target, and are left on this medium for an additional 18 hours after bombardment.

For particle bombardment, plasmid DNA (described above) is precipitated onto 1.8 mm tungsten particles using standard CaCl2-spermidine chemistry (see, for example, Klein et al., 1987, Nature 327:70-73). Each plate is bombarded once at 600 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, NY, pp 663-671.

Within 1-7 days after particle bombardment, the embryos are moved onto N6-based culture medium containing 3 mg/I of the selective agent bialaphos. Embryos, and later callus, are transferred to fresh selection plates every 2 weeks. The calli developing from the immature embryos are screened for the desired phenotype. After 6-8 weeks, transformed calli are recovered.

### B. Soybean Transformation

Soybean embryogenic suspension cultures are maintained in 35 ml liquid media SB196 or SB172 in 250 ml Erlenmeyer flasks on a rotary shaker, 150 rpm, 26 C with cool white fluorescent lights on 16:8 hr day/night photoperiod at light intensity of 30-35 uE/m2s. Cultures are subcultured every two weeks by inoculating approximately 35 mg of tissue into 35 ml of fresh liquid media. Alternatively, cultures are initiated and maintained in 6-well Costar plates.

SB 172 media is prepared as follows: (per liter), 1 bottle Murashige and Skoog Medium (Duchefa # M 0240), 1 ml B5 vitamins 1000× stock, 1 ml 2,4-D stock (Gibco 11215-019), 60 g sucrose, 2 g MES, 0.667 g L-Asparagine anhydrous (GibcoBRL 11013-026), pH 5.7. SB 196 media is prepared as follows: (per liter) 10 ml MS FeEDTA, 10 ml MS Sulfate, 10 ml FN-Lite Halides, 10 ml FN-Lite P,B,Mo, 1 ml B5 vitamins 1000× stock, 1 ml 2,4-D, (Gibco 11215-019), 2.83 g KNO3, 0.463 g (NH4)2SO4, 2 g MES, 1 g Asparagine Anhydrous, Powder (Gibco 11013-026), 10 g Sucrose, pH 5.8. 2,4-D stock concentration 10 mg/ml is prepared as follows: 2,4-D is solubilized in 0.1 N NaOH, filter-sterilized, and stored at −20° C. B5 vitamins 1000× stock is prepared as follows: (per 100 ml)—store aliquots at −20° C., 10 g myo-inositol, 100 mg nicotinic acid, 100 mg pyridoxine HCl, 1 g thiamin.

Soybean embryogenic suspension cultures are transformed with various plasmids by the method of particle gun bombardment (Klein et al., 1987 Nature 327:70. To prepare tissue for bombardment, approximately two flasks of suspension culture tissue that has had approximately 1 to 2 weeks to recover since its most recent subculture is placed in a sterile 60×20 mm petri dish containing 1 sterile filter paper in the bottom to help absorb moisture. Tissue (i.e. suspension clusters approximately 3-5 mm in size) is spread evenly across each petri plate. Residual liquid is removed from the tissue with a pipette, or allowed to evaporate to remove excess moisture prior to bombardment. Per experiment, 4-6 plates of tissue are bombarded. Each plate is made from two flasks.

To prepare gold particles for bombardment, 30 mg gold is washed in ethanol, centrifuged and resuspended in 0.5 ml of sterile water. For each plasmid combination (treatments) to be used for bombardment, a separate micro-centrifuge tube is prepared, starting with 50 μl of the gold particles prepared above. Into each tube, the following are also added; 5 μl of plasmid DNA (at 1 μg/μl), 50 μl CaCl2, and 20 μl 0.1 M spermidine. This mixture is agitated on a vortex shaker for 3 minutes, and then centrifuged using a microcentrifuge set at 14,000 RPM for 10 seconds. The supernatant is decanted and the gold particles with attached, precipitated DNA are washed twice with 400 μl aliquots of ethanol (with a brief centrifugation as above between each washing). The final volume of 100% ethanol per each tube is adjusted to 40 μl, and this particle/DNA suspension is kept on ice until being used for bombardment.

Immediately before applying the particle/DNA suspension, the tube is briefly dipped into a sonicator bath to disperse the particles, and then 5 μL of DNA prep is pipetted onto each flying disk and allowed to dry. The flying disk is then placed into the DuPont Biolistics PDS1000/HE. Using the DuPont Biolistic PDS1000/HE instrument for particle-mediated DNA delivery into soybean suspension clusters, the following settings are used. The membrane rupture pressure is 1100 psi. The chamber is evacuated to a vacuum of 27-28 inches of mercury. The tissue is placed approximately 3.5 inches from the retaining/stopping screen (3rd shelf from the bottom). Each plate is bombarded twice, and the tissue clusters are rearranged using a sterile spatula between shots.

Following bombardment, the tissue is re-suspended in liquid culture medium, each plate being divided between 2 flasks with fresh SB196 or SB172 media and cultured as described above. Four to seven days post-bombardment, the medium is replaced with fresh medium containing a selection agent. The selection media is refreshed weekly for 4 weeks and once again at 6 weeks. Weekly replacement after 4 weeks may be necessary if cell density and media turbidity is high.

Four to eight weeks post-bombardment, green, transformed tissue may be observed growing from untransformed, necrotic embryogenic clusters. Isolated, green tissue is removed and inoculated into 6-well microtiter plates with liquid medium to generate clonally-propagated, transformed embryogenic suspension cultures.

Each embryogenic cluster is placed into one well of a Costar 6-well plate with 5 mls fresh SB196 media with selection agent. Cultures are maintained for 2-6 weeks with fresh media changes every 2 weeks. When enough tissue is available, a portion of surviving transformed clones are subcultured to a second 6-well plate as a back-up to protect against contamination.

To promote in vitro maturation, transformed embryogenic clusters are removed from liquid SB196 and placed on solid agar media, SB 166, for 2 weeks. Tissue clumps of 2-4 mm size are plated at a tissue density of 10 to 15 clusters per plate. Plates are incubated in diffuse, low light (<10 μE) at 26+/−1° C. After two weeks, clusters are subcultured to SB 103 media for 3-4 weeks.

SB 166 is prepared as follows: (per liter), 1 pkg. MS salts (Gibco/BRL—Cat#11117-017), 1 ml B5 vitamins 1000× stock, 60 g maltose, 750 mg MgCl2 hexahydrate, 5 g activated charcoal, pH 5.7, 2 g gelrite. SB 103 media is prepared as follows: (per liter), 1 pkg. MS salts (Gibco/BRL—Cat#11117-017), 1 ml B5 vitamins 1000× stock, 60 g maltose, 750 mg MgCl2 hexahydrate, pH 5.7, 2 g gelrite. After 5-6 week maturation, individual embryos are desiccated by placing embryos into a 100×15 petri dish with a 1 cm2 portion of the SB103 media to create a chamber with enough humidity to promote partial desiccation, but not death.

Approximately 25 embryos are desiccated per plate. Plates are sealed with several layers of parafilm and again are placed in a lower light condition. The duration of the desiccation step is best determined empirically, and depends on size and quantity of embryos placed per plate. For example, small embryos or few embryos/plate require a shorter drying period, while large embryos or many embryos/plate require a longer drying period. It is best to check on the embryos after about 3 days, but proper desiccation will most likely take 5 to 7 days. Embryos will decrease in size during this process.

Desiccated embryos are planted in SB 71-1 or MSO medium where they are left to germinate under the same culture conditions described for the suspension cultures. When the plantlets have two fully-expanded trifoliate leaves, germinated and rooted embryos are transferred to sterile soil and watered with MS fertilizer. Plants are grown to maturity for seed collection and analysis. Healthy, fertile transgenic plants are grown in the greenhouse.

SB 71-1 is prepared as follows: 1 bottle Gamborg's B5 salts w/sucrose (Gibco/BRL—Cat#21153-036), 10 g sucrose, 750 mg MgCl2 hexahydrate, pH 5.7, 2 g gelrite. MSO media is prepared as follows: 1 pkg Murashige and Skoog salts (Gibco 11117-066), 1 ml B5 vitamins 1000× stock, 30 g sucrose, pH 5.8, 2 g Gelrite.

### C. Transformation of Maize Using Agrobacterium

Agrobacterium-mediated transformation of maize is performed essentially as described by Zhao et al., in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection and plant regeneration.

### 1. Immature Embryo Preparation:

Immature maize embryos are dissected from caryopses and placed in a 2 mL microtube containing 2 mL PHI-A medium.

### 2. Agrobacterium Infection and Co-Cultivation of Immature Embryos:

### 2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

### 2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is useful for recovering stable transgenic events.

### 3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

### 4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.
Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a validated Arabidopsis gene can be introduced into a maize inbred line either by direct transformation or introgression from a separately transformed line.

Transgenic plants, either inbred or hybrid, can undergo more vigorous field-based experiments to study expression effects

1. A method of improving an agronomic trait of a maize plant, the method comprising providing a transgenic maize plant comprising in its genome a recombinant DNA having at least one DNA element for modulating the expression of at least one target gene, wherein the at least one DNA element is selected from the group consisting of nucleotide sequences that are at least 90% identical to SEQ ID NOS: 1-197.

2. The method of claim 1, wherein the agronomic trait is drought tolerance.

3. The method of claim 1, wherein the agronomic trait is nitrogen use efficiency.

4. The method of claim 1, wherein the agronomic trait is yield increase.

5. The method of claim 1, wherein the DNA element modulates the expression of a target gene sequence selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703.

6. The method of claim 1 wherein the DNA element modulates the expression of a gene sequence encoding a target peptide sequence selected from the group consisting of SEQ ID NOS: 2497, 2499, 2505, 2507, 2514, 2516, 2526, 2530, 2536, 2542, 2623, 2634, 2676, 2753, 2831, 2832, 2888, 2892, 2895, 2943, 2947, 2955, 2975, and 3054.

7. A method of improving an agronomic trait of a maize plant, the method comprising providing a transgenic maize plant comprising in its genome a recombinant DNA for modulating the expression of at least one target gene, wherein the target gene sequence is selected from the group consisting of SEQ ID NOS: 1127-2495.

8. The method of claim 7, wherein the target gene sequence is selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703 and wherein the agronomic trait is one of drought tolerance, nitrogen use efficiency or yield.

9. The method of claim 7, wherein the target gene sequence is selected from the group consisting of SEQ ID NOS: 1168, 1178, 1179, 1185, 1194, 1220, 1710, 1716, 1733, 1738, 1771, 1784, 1795, 1807, 1823, 1872, 1892, 1926, 1936, 1937, 1938, 1942, 1970, 2001, 2003, 2006, 2026, 2074, 2105, 2109, 2110, 2130, 2145, 2152, 2174, 2175, 2189, 2192, 2199, 2200, 2202, 2240, 2245, 2246, 2291, 2299, 2310, 2313, 2340, 2341, 2371, 2412, 2413, 2414, 2417, 2429, 2430, 2431, 2443, 2468 and wherein the agronomic trait is one of nitrogen use efficiency or yield.

10. The method of claim 7, wherein the target gene sequence is selected from the group consisting of SEQ ID NOS: 1135, 1137, 1141, 1142, 1143, 1146, 1153, 1154, 1160, 1164, 1166, 1169, 1183, 1190, 1192, 1195, 1208, 1231, 1255, 1256, 1258, 1267, 1275, 1278, 1279, 1283, 1290, 1299, 1307, 1322, 1336, 1339, 1342, 1347, 1353, 1355, 1361, 1362, 1363, 1373, 1378, 1409, 1415, 1430, 1431, 1432, 1437, 1448, 1449, 1452, 1453, 1468, 1487, 1498, 1505, 1552, 1562, 1575, 1615, 1643, 1655, 1662, 1664, 1680, 1684 and wherein the agronomic trait is one of drought tolerance or yield.

11. (canceled)

12. (canceled)

13. (canceled)

14. (canceled)

15. An isolated polynucleotide comprising a microRNA selected from the group consisting of SEQ ID NOS: 1-197, wherein the microRNA modulates the expression of a target gene in maize involved in an agronomic trait, the target gene selected from the group consisting of SEQ ID NOS: 1128, 1130, 1136, 1138, 1145, 1147, 1157, 1161, 1167, 1173, 1254, 1265, 1308, 1342, 1390, 1471, 1472, 1533, 1537, 1540, 1588, 1592, 1600, 1605, 1621, and 1703.

16. A recombinant DNA construct comprising the polynucleotide of claim 15, wherein the DNA construct comprises a plant expressible regulatory element.

17. (canceled)

18. A transgenic maize plant comprising the DNA construct of claim 16.

19. A transgenic seed comprising the DNA construct of claim 16.

20-28. (canceled)
